SEQUENCE LISTING (103 sequences)

SEQ ID NO: 1 - protein, 94 aa, Artificial Sequence
  Description of Artificial Sequence Synthetic polypeptide
VSDVPRDLEVVAATPTSLLISWDAPAVTVRYYRITYGETGGNSPVQEFTVPGSKSTATISGLKPGVDYTITVYAVTGRGDSPASSKPISINYRT

SEQ ID NO: 2 - DNA, 5897 bp, Artificial Sequence
  Description of Artificial Sequence Synthetic polynucleotide
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatggctagcgtttctgatgttccgagggacctggaagttgttgctgcgacccccaccagcctactgatcagctggcttcaccatcgctctgacgtgcgctcttacaggatcacttacggagaaacaggaggaaatagccctgtccagaagttcactgtgcctgggtcgcgctccctggctaccatcagcggccttaaacctggagttgattataccatcactgtgtatgctgtcacttgggggtcttactgttgctctaatccaatttccattaattaccgaacagaaattgacaaaccatcccagggatccggaggcggttcaggcggaggtaaaggtggcggaggtaccgtttctgatgttccgagggacctggaagttgttgctgcgacccccaccagcctactgatcagctggtatcatcctttctattatgtcgcgcattcttacaggatcacttacggagaaacaggaggaaatagccctgtccaggagttcactgtgcctcgttcgccctggtttgctaccatcagcggccttaaacctggagttgattataccatcactgtgtatgctgtcactgatagtaacggttctcatccaatttccattaattaccgaacagaaattgacaaaccatcccaggagctcagatcccaccatcaccatcatcactgattaactaaacgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 3 - protein, 225 aa, Artificial Sequence
  Description of Artificial Sequence Synthetic polypeptide
MASVSDVPRDLEVVAATPTSLLISWLHHRSDVRSYRITYGETGGNSPVQKFTVPGSRSLATISGLKPGVDYTITVYAVTWGSYCCSNPISINYRTEIDKPSQGSGGGSGGGKGGGGTVSDVPRDLEVVAATPTSLLISWYHPFYYVAHSYRITYGETGGNSPVQEFTVPRSPWFATISGLKPGVDYTITVYAVTDSNGSHPISINYRTEIDKPSQELRSHHHHHH

SEQ ID NO: 4 - DNA, 309 bp, Artificial Sequence
  Description of Artificial Sequence Synthetic polynucleotide
gctagcgtttccgatgttccgagggacctggaggttgttgctgcgacccccaccagcctactgatcagctggttcgactacgctgtgacttattacaggatcacttacggagaaacaggaggaaatagccctgtccaggagttcactgtgcctggttggatctccactgctaccatcagcggccttaaacctggagttgattataccatcactgtgtatgctgtcactgacaactctcgttggccttttcgctctactccaatttccactaattaccgaacagaaattgacaaaccaccccagggatcc

SEQ ID NO: 5 - protein, 103 aa, Artificial Sequence
  Description of Artificial Sequence Synthetic polypeptide
ASVSDVPRDLEVVAATPTSLLISWFDYAVTYYRITYGETGGNSPVQEFTVPGWISTATISGLKPGVDYTITVYAVTDNSRWPFRSTPISTNYRTEIDKPPQGS

SEQ ID NO: 6 - DNA, 312 bp, Artificial Sequence
  Description of Artificial Sequence Synthetic polynucleotide
gctagcgtttctgatgttccgagggacctggaagttgttgctgcgacccccaccagcctactgatcagctggtacggtttttcgcttgcgagctcttacaggatcacttacggagaaacaggaggaaatagccctgtccaggagttcactgtgcctcgttcgccctggtttgctaccatcagcggccttaaacctggagttgattataccatcactgtgtatgctgtcacttctaacgacttttctaatcgttactctggtccaatttccattaattaccgaacagaaattgacaaaccatcccagggatcc

SEQ ID NO: 7 - protein, 104 aa, Artificial Sequence
  Description of Artificial Sequence Synthetic polypeptide
ASVSDVPRDLEVVAATPTSLLISWYGFSLASSYRITYGETGGNSPVQEFTVPRSPWFATISGLKPGVDYTITVYAVTSNDFSNRYSGPISINYRTEIDKPSQGS

SEQ ID NO: 8 - DNA, 312 bp, Artificial Sequence
  Description of Artificial Sequence Synthetic polynucleotide
gctagcgtttctgatgttccgagggacctggaagttgttgctgcgacccccaccagcctactgatcagctggtattttcgcgacccccggtacgtggactattacaggatcacttacggagaaacaggaggaaatagccctgcccaggagttcactgtgccttggtaccttcctgaggctaccatcagcggccttaaacccggagttgattataccatcactgtgtatgctgtcactggggacgatcagaatgctgggcttccaatttccattaattaccgaacagaaattgacaaaccatcccagggatcc

SEQ ID NO: 9 - protein, 104 aa, Artificial Sequence
  Description of Artificial Sequence Synthetic polypeptide
ASVSDVPRDLEVVAATPTSLLISWYFRDPRYVDYYRITYGETGGNSPAQEFTVPWYLPEATISGLKPGVDYTITVYAVTGDDQNAGLPISINYRTEIDKPSQGS

SEQ ID NO: 10 - DNA, 309 bp, Artificial Sequence
  Description of Artificial Sequence Synthetic polynucleotide
gctagcgtttctgatgttccgagggacctggaagttgttgctgcgacccccaccagcctactgatcagctggcttcaccatcgctctgacgtgcgctcttacaggatcacttacggagaaacaggaggaaatagccctgtccagaagttcactgtgcctgggtcgcgctccctggctaccatcagcggccttaaacctggagttgattataccatcactgtgtatgctgtcacttgggggtcttactgttgctctaatccaatttccattaattaccgaacagaaattgacaaaccatcccagggatcc

SEQ ID NO: 11 - protein, 103 aa, Artificial Sequence
  Description of Artificial Sequence Synthetic polypeptide
ASVSDVPRDLEVVAATPTSLLISWLHHRSDVRSYRITYGETGGNSPVQKFTVPGSRSLATISGLKPGVDYTITVYAVTWGSYCCSNPISINYRTEIDKPSQGS

SEQ ID NO: 12 - DNA, 318 bp, Artificial Sequence
  Description of Artificial Sequence Synthetic polynucleotide
gctagcgtttctgatgttccgagggacctggaagttgttgctgcgacccccaccagcctactgatcagctggtaccttcgtgacccccggtacgtggactattacaggatcacttacggagaaacaggaggaaatagccctgtccaggagttcactgtgccttggtaccttcctgaggctaccatcagcggccttaaacctggagttgattataccatcactgtgtatgctgtcacttacgatggctaccgcgagagtacccctctcccaatttccattaattaccgaacagaaattgacaaaccatcccagggatcc

SEQ ID NO: 13 - protein, 106 aa, Artificial Sequence
  Description of Artificial Sequence Synthetic polypeptide
ASVSDVPRDLEVVAATPTSLLISWYLRDPRYVDYYRITYGETGGNSPVQEFTVPWYLPEATISGLKPGVDYTITVYAVTYDGYRESTPLPISINYRTEIDKPSQGS

SEQ ID NO: 14 - DNA, 300 bp, Artificial Sequence
  Description of Artificial Sequence Synthetic polynucleotide
gctagcgtttctgatgttccgagggacctggaagttgttgctgcgacccccaccagcctactgatcagctggtatggttccagttacgcgtcctattacaggatcacttacggagaaacaggaggaaatagccctgtccaggagttcactgtgcctcgttcgccctggtttgctatcatcagcggcctgaaacctggagttgattataccatcactgtgtatgctgtcactcctagtgggatctctgctccaatttccattaattaccgaacagaaattgacaaaccatcccagggatcc

SEQ ID NO: 15 - protein, 100 aa, Artificial Sequence
  Description of Artificial Sequence Synthetic polypeptide
ASVSDVPRDLEVVAATPTSLLISWYGSSYASYYRITYGETGGNSPVQEFTVPRSPWFAIISGLKPGVDYTITVYAVTPSGISAPISINYRTEIDKPSQGS

SEQ ID NO: 16 - DNA, 312 bp, Artificial Sequence
  Description of Artificial Sequence Synthetic polynucleotide
gctagcgtttctgatgttccgagggacctggaagttgttgctgcgacccccaccagcctactgatcagctggtatcatcctttctattatgtcgcgcattcttacaggatcacttacggagaaacaggaggaaatagccctgtccaggagttcactgtgcctcgttcgccctggtttgctaccatcagcggccttaaacctggagttgattataccatcactgtgtatgctgtcactagtaagtgctatgatggttctgtcccaatttccattaattaccgaacagaaattgacaaaccatcccagggatcc

SEQ ID NO: 17 - protein, 104 aa, Artificial Sequence
  Description of Artificial Sequence Synthetic polypeptide
ASVSDVPRDLEVVAATPTSLLISWYHPFYYVAHSYRITYGETGGNSPVQEFTVPRSPWFATISGLKPGVDYTITVYAVTSKCYDGSVPISINYRTEIDKPSQGS

SEQ ID NO: 18 - DNA, 6800 bp, Artificial Sequence
  Description of Artificial Sequence Synthetic polynucleotide
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggttcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagcgtggagattgggctcgcacggctgacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagatgggttggagcctcatcttgctcttccttgtcgctgttgctcatatggctagcgtttctgatgttccgagggacctggaagttgttgctgcgacccccaccagcctactgatcagctggcttcaccatcgctctgacgtgcgctcttacaggatcacttacggagaaacaggaggaaatagccctgtccagaagttcactgtgcctgggtcgcgctccctggctaccatcagcggccttaaacctggagttgattataccatcactgtgtatgctgtcacttgggggtcttactgttgctctaatccaatttccattaattaccgaacagaaattgacaaaccatcccagggatccggaggtggcggtagtggcggaggtggttctacgcgtcaggtacaactgaagcagtcaggacctggcctagtgcagccctcacagagcctgtccatcacctgcacagtctctggtttctcattaactaactatggtgtacactgggttcgccagtctccaggaaagggtctggagtggctgggagtgatatggagtggtggaaacacagactataatacacctttcacatccagactgagcatcaacaaggacaattccaagagccaagttttctttaaaatgaacagtctgcaatctaatgacacagccatatattactgtgccagagccctcacctactatgattacgagtttgcttactggggccaagggaccctggtcaccgtttccgctgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgataagtcgacacgtgtgatcagatatcgcggccgctctagaccaggcgcctggatccagatcacttctggctaataaaagatcagagctctagagatctgtgtgttggttttttgtggatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcagcacagcaagggggaggattgggaagacaatagcaggcatgctggggatgcggtgggctctatgggtacctctctctctctctctctctctctctctctctctctctctcggtacctctctctctctctctctctctctctctctctctctctctcggtaccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 19 - DNA, 6801 bp, Artificial Sequence
  Description of Artificial Sequence Synthetic polynucleotide
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagcgtggagattgggctcgcacggctgacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagatgggttggagcctcatcttgctcttccttgtcgctgttgctacgcgtcaggtacaactgaagcagtcaggacctggcctagtgcagccctcacagagcctgtccatcacctgcacagtctctggtttctcattaactaactatggtgtacactgggttcgccagtctccaggaaagggtctggagtggctgggagtgatatggagtggtggaaacacagactataatacacctttcacatccagactgagcatcaacaaggacaattccaagagccaagttttctttaaaatgaacagtctgcaatctaatgacacagccatatattactgtgccagagccctcacctactatgattacgagtttgcttactggggccaagggaccctggtcaccgtttccgctgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggaggtggcggtagtggcggaggtggttctcatatggctagcgtttctgatgttccgagggacctggaagttgttgctgcgacccccaccagcctactgatcagctggcttcaccatcgctctgacgtgcgctcttacaggatcacttacggagaaacaggaggaaatagccctgtccagaagttcactgtgcctgggtcgcgctccctggctaccatcagcggccttaaacctggagttgattataccatcactgtgtatgctgtcacttgggggtcttactgttgctctaatccaatttccattaattaccgaacagaaattgacaaaccatcccagggatcctgataagtcgacacgtgtgatcagatatcgcggccgctctagaccaggcgcctggatccagatcacttctggctaataaaagatcagagctctagagatctgtgtgttggttttttgtggatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcagcacagcaagggggaggattgggaagacaatagcaggcatgctggggatgcggtgggctctatgggtacctctctctctctctctctctctctctctctctctctctctcggtacctctctctctctctctctctctctctctctctctctctctcggtaccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 20 - DNA, 6111 bp, Artificial Sequence
  Description of Artificial Sequence Synthetic polynucleotide
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagcgtggagattgggctcgcacggctgacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagatgagggtccccgctcagctcctggggctcctgctgctctggctcccaggtgcacatatggctagcgtttctgatgttccgagggacctggaagttgttgctgcgacccccaccagcctactgatcagctggcttcaccatcgctctgacgtgcgctcttacaggatcacttacggagaaacaggaggaaatagccctgtccagaagttcactgtgcctgggtcgcgctccctggctaccatcagcggccttaaacctggagttgattataccatcactgtgtatgctgtcacttgggggtcttactgttgctctaatccaatttccattaattaccgaacagaaattgacaaaccatcccagggatccggaggtggcggtagtggcggaggtggttcttcacgatgtgacatcctgctgacccagtctccagtcatcctgtctgtgagtccaggagaaagagtcagtttctcctgcagggccagtcagagtattggcacaaacatacactggtatcagcaaagaacaaatggttctccaaggcttctcataaagtatgcttctgagtctatctctggcatcccttccaggtttagtggcagtggatcagggacagattttactcttagcatcaacagtgtggagtctgaagatattgcagattattactgtcaacaaaataataactggccaaccacgttcggtgctgggaccaagctggagctcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaataggtcgacacgtgtgatcagatatcgcggccgctctagaccaggcgcctggatccagatcacttctggctaataaaagatcagagctctagagatctgtgtgttggttttttgtggatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcagcacagcaagggggaggattgggaagacaatagcaggcatgctggggatgcggtgggctctatgggtacctctctctctctctctctctctctctctctctctctctctcggtacctctctctctctctctctctctctctctctctctctctctcggtaccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 21 - DNA, 6108 bp, Artificial Sequence
  Description of Artificial Sequence Synthetic polynucleotide
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagcgtggagattgggctcgcacggctgacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagatgagggtccccgctcagctcctggggctcctgctgctctggctcccaggtgcacgatgtgacatcctgctgacccagtctccagtcatcctgtctgtgagtccaggagaaagagtcagtttctcctgcagggccagtcagagtattggcacaaacatacactggtatcagcaaagaacaaatggttctccaaggcttctcataaagtatgcttctgagtctatctctggcatcccttccaggtttagtggcagtggatcagggacagattttactcttagcatcaacagtgtggagtctgaagatattgcagattattactgtcaacaaaataataactggccaaccacgttcggtgctgggaccaagctggagctcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtggaggtggcggtagtggcggaggtggttctcatatggctagcgtttctgatgttccgagggacctggaagttgttgctgcgacccccaccagcctactgatcagctggcttcaccatcgctctgacgtgcgctcttacaggatcacttacggagaaacaggaggaaatagccctgtccagaagttcactgtgcctgggtcgcgctccctggctaccatcagcggccttaaacctggagttgattataccatcactgtgtatgctgtcacttgggggtcttactgttgctctaatccaatttccattaattaccgaacagaaattgacaaaccatcccagggatcctaataggtcgacacgtgtgatcagatatcgcggccgctctagaccaggcgcctggatccagatcacttctggctaataaaagatcagagctctagagatctgtgtgttggttttttgtggatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcagcacagcaagggggaggattgggaagacaatagcaggcatgctggggatgcggtgggctctatgggtacctctctctctctctctctctctctctctctctctctctctcggtacctctctctctctctctctctctctctctctctctctctctcggtaccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 22 - protein, 9 aa, Homo sapiens
DAPAVTVRY

SEQ ID NO: 23 - protein, 5 aa, Homo sapiens
GSKST

SEQ ID NO: 24 - protein, 10 aa, Homo sapiens
GRGDSPASSK

SEQ ID NO: 25 - protein, 8 aa, Artificial Sequence
  Description of Artificial Sequence Synthetic peptide
YGFSLASS

SEQ ID NO: 26 - protein, 5 aa, Artificial Sequence
  Description of Artificial Sequence Synthetic peptide
RSPWF

SEQ ID NO: 27 - protein, 10 aa, Artificial Sequence
  Description of Artificial Sequence Synthetic peptide
SNDFSNRYSG

SEQ ID NO: 28 - protein, 7 aa, Artificial Sequence
  Description of Artificial Sequence Synthetic peptide
FDYAVTY

SEQ ID NO: 29 - protein, 5 aa, Artificial Sequence
  Description of Artificial Sequence Synthetic peptide
GWIST

SEQ ID NO: 30 - protein, 10 aa, Artificial Sequence
  Description of Artificial Sequence Synthetic peptide
DNSHWPFRST

SEQ ID NO: 31 - protein, 10 aa, Artificial Sequence
  Description of Artificial Sequence Synthetic peptide
YLRDPRYVDY

SEQ ID NO: 32 - protein, 5 aa, Artificial Sequence
  Description of Artificial Sequence Synthetic peptide
WYLPE

SEQ ID NO: 33 - protein, 10 aa, Artificial Sequence
  Description of Artificial Sequence Synthetic peptide
YDGYRESTPL

SEQ ID NO: 34 - protein, 10 aa, Artificial Sequence
  Description of Artificial Sequence Synthetic peptide
YGPFYYVAHS

SEQ ID NO: 35 - protein, 8 aa, Artificial Sequence
  Description of Artificial Sequence Synthetic peptide
SKCYDGSV

SEQ ID NO: 36 - protein, 10 aa, Artificial Sequence
  Description of Artificial Sequence Synthetic peptide
YHPFYYVAHS

SEQ ID NO: 37 - protein, 6 aa, Artificial Sequence
  Description of Artificial Sequence Synthetic peptide
DSNGSH

SEQ ID NO: 38 - protein, 8 aa, Artificial Sequence
  Description of Artificial Sequence Synthetic peptide
YGSSYASY

SEQ ID NO: 39 - protein, 6 aa, Artificial Sequence
  Description of Artificial Sequence Synthetic peptide
PSGISA

SEQ ID NO: 40 - protein, 9 aa, Artificial Sequence
  Description of Artificial Sequence Synthetic peptide
LHHRSDVRS

SEQ ID NO: 41 - protein, 5 aa, Artificial Sequence
  Description of Artificial Sequence Synthetic peptide
GSRSL

SEQ ID NO: 42 - protein, 8 aa, Artificial Sequence
  Description of Artificial Sequence Synthetic peptide
WGSYCCSN

SEQ ID NO: 43 - protein, 10 aa, Artificial Sequence
  Description of Artificial Sequence Synthetic peptide
YFRDPRYVDY

SEQ ID NO: 44 - protein, 5 aa, Artificial Sequence
  Description of Artificial Sequence Synthetic peptide
WYLPE

SEQ ID NO: 45 - protein, 8 aa, Artificial Sequence
  Description of Artificial Sequence Synthetic peptide
GDDQNAGL

SEQ ID NO: 46 - protein, 8 aa, Artificial Sequence
  Description of Artificial Sequence Synthetic peptide
CTHLHWDY

SEQ ID NO: 47 - protein, 5 aa, Artificial Sequence
  Description of Artificial Sequence Synthetic peptide
ALCPG

SEQ ID NO: 48 - protein, 6 aa, Artificial Sequence
  Description of Artificial Sequence Synthetic peptide
VGGDDW

SEQ ID NO: 49 - protein, 7 aa, Artificial Sequence
  Description of Artificial Sequence Synthetic peptide
DMPFSDS

SEQ ID NO: 50 - protein, 5 aa, Artificial Sequence
  Description of Artificial Sequence Synthetic peptide
GTDSL

SEQ ID NO: 51 - protein, 7 aa, Artificial Sequence
  Description of Artificial Sequence Synthetic peptide
SSGSNSY

SEQ ID NO: 52 - protein, 10 aa, Artificial Sequence
  Description of Artificial Sequence Synthetic peptide
YCPDGCHSYY

SEQ ID NO: 53 - protein, 5 aa, Artificial Sequence
  Description of Artificial Sequence Synthetic peptide
RSISS

SEQ ID NO: 54 - protein, 6 aa, Artificial Sequence
  Description of Artificial Sequence Synthetic peptide
FRWPSF

SEQ ID NO: 55 - protein, 9 aa, Artificial Sequence
  Description of Artificial Sequence Synthetic peptide
NTYFSFLYY

SEQ ID NO: 56 - protein, 5 aa, Artificial Sequence
  Description of Artificial Sequence Synthetic peptide
SSLHT

SEQ ID NO: 57 - protein, 6 aa, Artificial Sequence
  Description of Artificial Sequence Synthetic peptide
GTWPSY

SEQ ID NO: 58 - protein, 10 aa, Artificial Sequence
  Description of Artificial Sequence Synthetic peptide
SYSSYNSWDS

SEQ ID NO: 59 - protein, 5 aa, Artificial Sequence
  Description of Artificial Sequence Synthetic peptide
NSDCI

SEQ ID NO: 60 - protein, 8 aa, Artificial Sequence
  Description of Artificial Sequence Synthetic peptide
RDCDFYSY

SEQ ID NO: 61 - protein, 9 aa, Artificial Sequence
  Description of Artificial Sequence Synthetic peptide
YYHLRGLDS

SEQ ID NO: 62 - protein, 5 aa, Artificial Sequence
  Description of Artificial Sequence Synthetic peptide
RSYST

SEQ ID NO: 63 - protein, 7 aa, Artificial Sequence
  Description of Artificial Sequence Synthetic peptide
VNDYISY

SEQ ID NO: 64 - protein, 9 aa, Artificial Sequence
  Description of Artificial Sequence Synthetic peptide
SSSLYNSAY

SEQ ID NO: 65 - protein, 5 aa, Artificial Sequence
  Description of Artificial Sequence Synthetic peptide
VWDCT

SEQ ID NO: 66 - protein, 7 aa, Artificial Sequence
  Description of Artificial Sequence Synthetic peptide
PNYSFSL

SEQ ID NO: 67 - protein, 8 aa, Artificial Sequence
  Description of Artificial Sequence Synthetic peptide
CCLFFSGY

SEQ ID NO: 68 - protein, 5 aa, Artificial Sequence
  Description of Artificial Sequence Synthetic peptide
GLVYW

SEQ ID NO: 69 - protein, 6 aa, Artificial Sequence
  Description of Artificial Sequence Synthetic peptide
DNVGSN

SEQ ID NO: 70 - protein, 8 aa, Artificial Sequence
  Description of Artificial Sequence Synthetic peptide
SFPCVSSS

SEQ ID NO: 71 - protein, 5 aa, Artificial Sequence
  Description of Artificial Sequence Synthetic peptide
GDTTS

SEQ ID NO: 72 - protein, 7 aa, Artificial Sequence
  Description of Artificial Sequence Synthetic peptide
STCYPSY

SEQ ID NO: 73 - protein, 10 aa, Artificial Sequence
  Description of Artificial Sequence Synthetic peptide
SCPICPRATS

SEQ ID NO: 74 - protein, 4 aa, Artificial Sequence
  Description of Artificial Sequence Synthetic peptide
ATSS

SEQ ID NO: 75 - protein, 8 aa, Artificial Sequence
  Description of Artificial Sequence Synthetic peptide
DQGYDDSA

SEQ ID NO: 76 - protein, 9 aa, Artificial Sequence
  Description of Artificial Sequence Synthetic peptide
QCHYYYAQS

SEQ ID NO: 77 - protein, 5 aa, Artificial Sequence
  Description of Artificial Sequence Synthetic peptide
SSKST

SEQ ID NO: 78 - protein, 10 aa, Artificial Sequence
  Description of Artificial Sequence Synthetic peptide
YNWFLDSVSI

SEQ ID NO: 79 - protein, 8 aa, Artificial Sequence
  Description of Artificial Sequence Synthetic peptide
GAPACAAY

SEQ ID NO: 80 - protein, 5 aa, Artificial Sequence
  Description of Artificial Sequence Synthetic peptide
GSGTS

SEQ ID NO: 81 - protein, 8 aa, Artificial Sequence
  Description of Artificial Sequence Synthetic peptide
SRYYYCSE

SEQ ID NO: 82 - protein, 9 aa, Artificial Sequence
  Description of Artificial Sequence Synthetic peptide
CCSDNCSNS

SEQ ID NO: 83 - protein, 5 aa, Artificial Sequence
  Description of Artificial Sequence Synthetic peptide
RSCFM

SEQ ID NO: 84 - protein, 6 aa, Artificial Sequence
  Description of Artificial Sequence Synthetic peptide
DSNGPH

SEQ ID NO: 85 - protein, 15 aa, Artificial Sequence
  Description of Artificial Sequence Synthetic peptide
GSGGGSGGGKGGGGT

SEQ ID NO: 86 - protein, 15 aa, Artificial Sequence
  Description of Artificial Sequence Synthetic peptide
GGGGSGGGGSGGGGS

SEQ ID NO: 87 - protein, 10 aa, Artificial Sequence
  Description of Artificial Sequence Synthetic peptide
GGGGSGGGGS

SEQ ID NO: 88 - protein, 7 aa, Artificial Sequence
  Description of Artificial Sequence Synthetic peptide
EIDKSPQ

SEQ ID NO: 89 - protein, 11 aa, Artificial Sequence
  Description of Artificial Sequence Synthetic peptide
HHHHHHKGSGK

SEQ ID NO: 90 - protein, 22 aa, Artificial Sequence
  Description of Artificial Sequence Synthetic peptide
EIDKPSQGSGGGSGGGKGGGGT

SEQ ID NO: 91 - protein, 17 aa, Artificial Sequence
  Description of Artificial Sequence Synthetic peptide
EIDKPSQELRSHHHHHH

SEQ ID NO: 92 - protein, 4 aa, Artificial Sequence
  Description of Artificial Sequence Synthetic peptide
GSGT

SEQ ID NO: 93 - protein, 27 aa, Artificial Sequence
  Description of Artificial Sequence Synthetic peptide
GSGGGSGGGKGGGSGGGNGGGSGGGGT

SEQ ID NO: 94 - protein, 6 aa, Artificial Sequence
  Description of Artificial Sequence Synthetic 6xHis tag
HHHHHH

SEQ ID NO: 95 - protein, 8 aa, Homo sapiens
DAPAVTVR

SEQ ID NO: 96 - protein, 5 aa, Artificial Sequence
  Description of Artificial Sequence Synthetic peptide
GGGGG

SEQ ID NO: 97 - protein, 5 aa, Artificial Sequence
  Description of Artificial Sequence Synthetic peptide
LPETG

SEQ ID NO: 98 - protein, 12 aa, Homo sapiens
  MOD_RES (5)..(5): Any amino acid
WDAPXAVTVRYY

SEQ ID NO: 99 - protein, 7 aa, Homo sapiens
PGSKSTA

SEQ ID NO: 100 - protein, 12 aa, Homo sapiens
TGRGDSPASSKP

SEQ ID NO: 101 - DNA, 16 bp, Artificial Sequence
  Description of Artificial Sequence Synthetic oligonucleotide
ttaactaaacgagatc

SEQ ID NO: 102 - protein, 10 aa, Artificial Sequence
  Description of Artificial Sequence Synthetic peptide
ELRSHHHHHH

SEQ ID NO: 103 - protein, 11 aa, Homo sapiens
WDAPAVTVRYY

(19) United States
(12) Patent Application Publication
(10) Pub. No.: US 2012/0270797 A1
(43) Pub. Date: Oct. 25, 2012

(54) ENGINEERED PROTEINS INCLUDING MUTANT FIBRONECTIN DOMAINS

(75) Inventors: Karl Dane Wittrup, Chestnut Hill, MA (US); Jamie B. Spangler, Palo Alto, CA (US); Benjamin Joseph Hackel, Edina, MN (US)

(73) Assignee: Massachusetts Institute of Technology, Cambridge, MA (US)

(21) Appl. No.: 13/390,086

(22) PCT Filed: Aug. 13, 2010
(86) PCT No.: PCT/US2010/045490

§ 371 Date: Jul. 2, 2012

Related U.S. Application Data
(60) Provisional application No. 61/233,820, filed on Aug. 13, 2009.
(60) Provisional application No. 61/370,377, filed on Aug. 3, 2010.

(51) Int. Cl. C07K 19/00; C12N 15/62; C12N 15/63; C12N 1/19; A61P 35/00; C12N 1/21; C12N 5/10; C12N 9/96; A61K 38/17

(52) U.S. Cl. 514/19.3; 530/395; 530/387.3; 435/188; 536/23.4; 435/320.1; 435/252.3; 435/348; 435/254.2; 435/325

(57) ABSTRACT

The present invention features engineered proteins that can include a genetically modified Fn domain; two or more such domains joined to one another; or at least one genetically modified Fn domain joined to a target-specific protein scaffold. One or more accessory sequences can be included in or added to any of these configurations. Methods of use, including methods of treating cancer, with the engineered proteins are also disclosed.

### CROSS-REFERENCE TO RELATED APPLICATIONS

This application claims the benefit of the filing date of U.S. provisional application No. 61/233,820, filed Aug. 13, 2009, and U.S. provisional application No. 61/370,377, filed Aug. 3, 2010. For the purpose of any U.S. patent that may grant based on the present application, the content of these prior provisional applications is incorporated herein by reference in its entirety.

### GOVERNMENT RIGHTS STATEMENT

This invention was made with government support awarded by the National Institutes of Health under Grant No. CA96504 and National Science Foundation Fellowship Stipend 2387941. The U.S. government has certain rights in this invention.

### TECHNICAL FIELD

This invention relates to engineered proteins, and more particularly to engineered proteins that include at least one genetically modified fibronectin (Fn) domain. The proteins can specifically bind target molecules, such as cell surface receptors, and thereby affect cellular physiology (e.g., cellular proliferation, differentiation, or migration).

### SUMMARY OF THE INVENTION

The present invention is based, in part, on our discovery of engineered proteins that include at least one genetically modified fibronectin (Fn) domain (e.g., a type III fibronectin domain (Fn3)). Where more than one domain is included, each domain may bind a different epitope on a given molecular target. For example, an engineered protein can include (a) a first genetically modified Fn domain that binds a first epitope on a molecular target (e.g., a cellular receptor) and (b) a second genetically modified Fn domain that binds a second epitope on the same target (e.g., the same cellular receptor).

In one embodiment, the engineered protein can include (a) one or more genetically modified Fn domains and (b) one or more heterologous amino acid sequences, which may contribute to the therapeutic activity of the engineered protein by, for example, binding an epitope on the molecular target. We may refer to such heterologous amino acid sequences as target-specific protein scaffolds. While heterologous sequences (or target-specific protein scaffolds) are described further below, we note here that they can constitute an immunoglobulin or a biologically active fragment or other variant thereof (e.g., an scFv). More broadly, we use the term “heterologous” to indicate that the amino acid sequences that may contribute to therapeutic activity are distinct (e.g., distinct in their sequence or structure) from the genetically modified Fn domain to which they are joined.

Any of the engineered proteins can further include an amino acid sequence that: prolongs the circulating half-life of the engineered protein; facilitates its purification; facilitates conjugation; is a label, marker or tag (including an imaging agent) or serves as a linker (e.g., between a first and second genetically modified Fn domain or between a genetically modified Fn domain and a heterologous amino acid sequence such as an immunoglobulin). We may refer to these sequences as “accessory” sequences.

To summarize the embodiments described above, the engineered protein can be: a genetically modified Fn domain; two or more such domains joined to one another; or at least one genetically modified Fn domain joined to a target-specific protein scaffold. One or more accessory sequences can be included in or added to any of these configurations. While we discuss these proteins further below, we note here that where at least one genetically modified Fn domain is joined to a target-specific protein scaffold, the protein scaffold can be an immunoglobulin (e.g., an IgG) that is joined (directly or via a linker) to one, two, or more genetically modified Fn domains. The Fn domains can be identical to one another or distinct, and they can be joined to either the amino or carboxy terminus of the target-specific protein scaffold. For example, where the protein scaffold is an IgG, one or more genetically modified Fn domains can be joined (e.g., fused) to the amino or carboxy terminus of a light chain (or chains), to the amino or carboxy terminal of a heavy chain (or chains), or to any combination of these positions. For example, a first genetically modified Fn domain can be joined to the amino terminus of one or both heavy chains and a second genetically modified Fn domain can be fused to the carboxy terminus of one or both light chains. The first and second Fn domains can be the same in their sequence and/or binding specificity (e.g., they may bind the same epitope on a molecular target) or they may differ from one another in their sequence and/or binding specificity (e.g., they may bind two different epitopes on the same or different molecular targets).

Where an engineered protein binds more than one epitope, we may refer to the engineered protein as “heterovalent” (e.g., heterobivalent where two different epitopes are bound; heterotrivaent where three different epitopes are bound; and so forth). Where an engineered protein binds two of the same epitope, we may refer to it as homobivalent. We may also refer to the binding as “specific” or “selective”, as a genetically modified Fn domain or a target-specific protein scaffold (e.g., an immunoglobulin) can bind an epitope on a molecular target to the substantial exclusion of other molecular targets or other epitopes within the same target.

We may refer to the engineered proteins described herein as “including” certain sequences. For example, we describe engineered proteins including first and second genetically modified fibronectin domains. We also describe proteins including first and second genetically modified fibronectin domains and a heterologous amino acid sequence. In all events, the engineered proteins described herein can include, consist of, or consist essentially of the recited sequences.

The engineered proteins, compositions containing them pharmaceutically acceptable preparations, stock solutions, kits, and the like), nucleic acids encoding them, and cells in which they are expressed (e.g., cells in tissue culture) are all within the scope of the present invention. Methods of making and methods of isolating or purifying the engineered proteins are also within the scope of the present invention. We may refer to an engineered protein as “isolated” or “purified” when it has been substantially separated from materials with which it was previously associated. For example, an engineered protein can be isolated or purified following chemical synthesis or expression in cell culture. Methods of using the engineered proteins to assess cells in vitro and to treat patients are also within the scope of the present invention. Production, isolation, formulation, screening, diagnostic and treatment methods are discussed further below.

The genetically modified Fn domains, heterologous sequences, and accessory sequences can be joined by various means, including by covalent bonds. For example, these sequences can be joined as a fusion protein (e.g., where amino acid residues are joined by peptide bonds) or as a chemical conjugate. As noted, the accessory sequence can be a polypeptide linker between two Fn domains or between a Fn domain and a heterologous sequence. For example, the engineered protein can consist of or include two genetically modified Fn domains that are fused to one another or conjugated to one another. In another embodiment, the engineered protein can consist of or include one or more genetically modified Fn domains that are fused to or conjugated with an antibody targeting the same molecular target (or antigen) such as Erbitux® (cetuximab; Imclone), Vectibix® (panitumumab; Amgen), EMD72000 (EMD Serono), antibody 806 (The Ludwig Institute for Cancer Research), or antibody 425 (Merck). A genetically modified Fn domain and a target-specific protein scaffold (e.g., an immunoglobulin) target the same molecular target (or antigen) when they specifically bind the same molecular target (or antigen). For example, the genetically modified Fn domain and a target-specific protein scaffold to which it is joined can specifically bind the same cell-surface protein (e.g., a tyrosine kinase receptor). The genetically modified Fn domain and the target-specific protein scaffold may bind distinct (e.g., non-overlapping) epitopes on the molecular target.

We may refer to antibodies such as those listed above, any of which can be incorporated into the present engineered proteins, as “ligand-competitive antibodies.” While one or more genetically modified Fn domains can be joined to (e.g., fused to or conjugated with) a whole, complete, or full-length protein scaffold, the Fn domain(s) can also be joined to a biologically or therapeutically active fragment or other variant of a protein scaffold (e.g., an antibody or another target-specific protein scaffold, examples of which are provided below). Thus, fragments or other variants of the currently available antibodies listed above can also be incorporated into the engineered proteins of the present invention and are useful in the present methods so long as they retain biological activity (e.g., sufficient and selective binding to the molecular target).

Compositions in which two or more of the amino acid sequences described herein are included but not physically joined are also within the scope of the present invention. For example, the composition can be a pharmaceutically acceptable preparation including, in admixture, a genetically modified fibronectin domain and a heterologous amino acid sequence. For example, the composition can be a solution suitable for intravenous administration. Similarly, cells and patients can be treated as described herein but with an admixture or similar formulation of two or more of the target-binding amino acid sequences of the engineered proteins described herein. For example, a pharmaceutical formulation can include, as separate entities, a genetically modified Fn domain and an immunoglobulin, including any of the currently available immunoglobulins that specifically bind a molecular target as described herein (e.g., cetuximab).

In other aspects, the invention features methods of making the engineered proteins described herein and compositions containing them (e.g., stock solutions or pharmaceutically acceptable formulations). The methods of generating engineered proteins can be carried out using standard techniques known in the art. For example, one can use standard methods of protein expression (e.g., expression in cell culture with recombinant vectors) followed by purification from the expression system. In some circumstances (e.g., to produce a given domain, linker, or tag), chemical synthesis can also be used. These methods can be used alone or in combination to produce engineered proteins having one or more of the sequences described in detail herein as well as engineered proteins that differ from those proteins but that have the structure and one or more functions of an engineered protein as described herein (e.g., the configuration and components described herein and an ability to specifically bind a molecular target).

In another aspect, the invention features screening methods in which one or more epitopes on a target are used to identify or construct engineered proteins (or domains thereof) that specifically bind that epitope or epitopes.

Among the process methods of the present invention are methods of creating combinatorial libraries of fibronectin clones, taking into consideration the parameters specified in the Examples below. The libraries may include clones in which one or more of the amino acid residues in the otherwise diversified binding loops of a Fn domain are maintained as wild-type sequence or as preferentially biased toward wild-type sequence. The selection of these conserved or biased amino acid positions can be aided through identification of clones that stabilize the domain or are accessible to solvent based on structural analysis. The clones may also be present preferentially in Fn domains of various species, and the present methods can include a step in which an alignment is carried out as described in the Examples below. The library may be biased toward clones having amino acids that are better suited for molecular recognition (e.g., tyrosine, serine, and glycine). In particular, amino acids observed in natural binding repertoires may be used. These combinatorial libraries may be constructed from degenerate nucleotides that produce the desired amino acid bias. These libraries may contain a higher fraction of functional sequences than results from fully random library generation. Libraries made by the methods described herein are within the scope of the present invention as are methods of screening such libraries to identify clones that can be incorporated in an engineered protein.

To identify genetically modified Fn domains, one can diversify a domain by mutating the DNA encoding one or more residues in the BC, DE, and/or FG loops (as defined in the art; see, e.g., Ruoslahti, Ann. Rev. Biochem. 57:375-413, 1988). While useful Fn domains are described further below, we note here that they can be variants (e.g., mutants) of a type III domain and, more specifically, of the tenth type III domain. Virtually any Fn domain may serve as the original source of the genetically modified Fn domain that becomes incorporated into the present proteins. For example, the Fn domain may have a sequence modified from a mammalian (e.g., human) Fn domain. The diversification process may also be combined with homologous recombination of mutated loop gene fragments in which the constant portion of the Fn gene is used as a homologous region for recombination. This approach may be used in parallel with mutation of the entire Fn gene including the constant region. These approaches enable the creation of broader sequence diversity including mutations to either or both of the constant and loop regions.

The engineered proteins are not limited to those that affect cellular physiology by any particular mechanism. Our work to date indicates that antibody-Fn fusions are able to cluster cellular receptors on the cell surface. For example, we have fused the clinically approved human monoclonal antibody (mAb) 225 (cetuximab) with variants of the tenth type III domain of human fibronectin that recognize the EGF receptor (EGFR) to establish multispecific antibody-fibronectin fusions capable of clustering EGFR. These constructs induce receptor clustering and effectively downregulate EGFR in a number of cancerous cell lines without agonizing signaling. The engineered proteins of the present invention may, therefore, bring about this same downregulation. We have also concluded that the antibody constant domain can aid in the persistence of the proteins in the bloodstream and enhance immune cell recruitment. Thus, the amino acid sequence that prolongs the circulating half-life may be a part of the immunoglobulin portion of immunoglobulin-fibronectin fusions. The modular structure and design of the present proteins forms the basis for a new generation of therapeutics, including antibody-based therapeutics, that can bind to different (e.g., nonoverlapping) regions on molecular targets, including cell-surface targets (e.g., cellular receptors such as a receptor tyrosine kinase).

In use, for example when an engineered protein is brought into contact with a cell expressing a target molecule (e.g., a cell in vivo or in cell or tissue culture), the engineered protein may cause a substantial decrease in the amount of the target (e.g., an EGFR or other receptor tyrosine kinase) on the surface of the cell. We expect this downregulation to occur without prompting significant activation of the target. For example, where the molecular target is a cell surface receptor, the engineered protein can downregulate the receptor without activating the receptor's signaling cascade. As a result, one can bring about a desired change in cellular physiology. For example, an engineered protein targeting the EGFR may inhibit cellular proliferation or migration. As such, these proteins are therapeutically useful (e.g., in treating cancers involving EGF receptor-positive cells). Engineered proteins that target an EGFR (including a constitutively active mutant such as EGFRvIII) can be used in treating any of the same cancers presently treated with EGFR antagonists. Specific cancers amenable to treatment with proteins that target the EGFR include breast cancer, bladder cancer, non-small-cell lung cancer, colorectal cancer, squamous-cell carcinoma of the head and neck, ovarian cancer, cervical cancer, lung cancer, esophageal cancer, glioblastomas, and pancreatic cancer. By targeting other cell-surface proteins, one can treat other types of cancers. Those of ordinary skill in the art will appreciate which molecular targets are associated with which cancers or other diseases, disorders, or conditions.

In other methods, the engineered proteins can be used, due to their target specificity, to deliver cargo (e.g., a therapeutic agent) to a cell that expresses the target molecule. In this event, the target may or may not be a receptor; any cell-surface, cancer-specific protein can be targeted. Further, as the proteins can be internalized, the delivery can encompass an intracellular delivery of the cargo. The cargo can vary widely and includes nucleic acids (e.g., antisense oligonucleotides, microRNAs, and any nucleic acid that mediates RNAi (e.g., an siRNA or shRNA)). The cargo can also be a conventional small molecule therapeutic agent, such as a chemotherapeutic agent or any agent that is toxic to the cell to which it is delivered (e.g., a radioisotope).

In any of the methods of treatment, the subject can be a human and the method can include a step of identifying a patient for treatment (e.g., by performing a diagnostic assay for a cancer). Further, one may obtain a biological sample from a patient and expose cancerous cells within the sample to one or more engineered proteins ex vivo to determine whether or to what extent the engineered protein downregulates a target expressed by the cells or inhibits their proliferation or capacity for metastasis. Similarly, one may obtain a biological sample from a patient and expose cancerous cells within the sample to one or more of the present proteins that have been engineered to carry toxic cargo. Evaluating cell survival or other parameters (e.g., cellular proliferation or migration) can yield information that reflects how well a patient's cancer may respond to in vivo treatment with the engineered protein tested in culture.

While the engineered proteins can contain naturally occurring amino acid residues (and may consist of only naturally occurring amino acid residues), the invention is not so limited. The proteins can also include non-naturally occurring residues. Any of the engineered proteins may also vary (either from each other or from a wild-type protein from which they were derived) due to post-translational modification(s). For example, the glycosylation pattern may vary or there may be differences in amidation or phosphorylation.

Within a given engineered protein, the sequence of the first Fn domain and the sequence of the second Fn domain can vary from one another in the regions that confer epitope binding specificity but be otherwise identical or nearly identical (e.g., at least 90% identical). For example, the first domain and the second domain can be generated from a type III Fn domain (e.g., a tenth type III Fn domain) and can vary from either one another or from the wild type sequence from which they were derived in one or more of the regions defining the BC loop, the DE loop, and the FG loop. Aside from the variability in these regions, the first Fn domain and the second Fn domain can be identical to one another or nearly identical (e.g., at least 90%, 95%, or 98% identical). In any event, the Fn domain engineered (e.g., mutated) can be a human or other mammalian Fn domain.

The variability (i.e., variability between one genetically modified Fn domain and another or between such a domain and the wild type sequence from which it was derived) can be generated by the addition, deletion or substitution of amino acid residues. A first genetically modified Fn domain and a second genetically modified Fn domain can be at least or about 40%, 50%, 60%, 70%, 75%, 80%, 85%, 90%, 95%, 98%, or 99% identical. A genetically modified Fn domain and the wild-type sequence from which it was derived can be at least or about 40%, 50%, 60%, 70%, 75%, 80%, 85%, 90%, 95%, 98%, or 99% identical.

More specifically, a Fn domain included in an engineered protein can be generated from the following wild-type fibronectin domain, where residues 23-31 (underlined) represent the BC loop, residues 52-56 (also underlined) represent the DE loop, and residues 77-86 (also underlined) represent the FG loop. Residues within one or more of the loops can be engineered, and the remaining residues, which constitute the constant region, can be also varied or invariant: VSDVPRDLEVVAATPTSLLISWDAPAVTVRYYRITYGETGGNSPVQEFTVPGSKSTATIS GLKPGVDYTITVYAVTGRGDSPASSKPISINYRT (SEQ ID NO:1)

As noted, residues within the loop regions can be altered to effect a change in epitope-binding specificity (specific mutations are described further below), and the constant region can remain unchanged or vary from one Fn domain to another as described herein.

Previously, receptor downregulation has been achieved using multiple receptor-targeted antibodies, but the current technology enables downregulation with a single agent. This may be advantageous for clinical development and efficacy. The present invention is exemplified by our work with the EGF receptor. As two EGFR-targeted antibodies are approved for clinical use in oncology, the EGFR has been validated as a therapeutic target.

The method of treatment claims included herein may be expressed in terms of “use.” For example, the present invention features the use of the engineered proteins described herein in the treatment of cancer or in the manufacture of a medicament for the treatment of cancer.

The details of one or more embodiments of the invention are set forth in the accompanying drawings, the description below, and/or the claims. Other features, objects, and advantages of the invention will be apparent from the drawings, descriptions, and claims.

### BRIEF DESCRIPTION OF THE DRAWINGS

FIG. 1(A) and FIG. 1(B) depict the results of analyses of sequences within wild-type Fn3 domains (Panel A) and genetically modified Fn domains (Panel B). The “x” in the BC loop corresponds to an amino acid present in other domains that is not present in the human tenth type III domain. The outline around S81-S84 represents rare positions as most type III domains contain shorter FG loops. In Panel (B), the amino acid frequency at each position was compared to the frequency in the composite naïve libraries.

FIG. 2 is a bar graph mapping amino acid distributions. The frequencies of each amino acid in multiple distributions are presented. NNB refers to a degenerate codon with 25% of each nucleotide at the first two positions and 33% of C, T, and G at the third position. Tyr/Ser refers to an even mix of tyrosine and serine. CDR-H3 refers to the expressed human and mouse CDR-H3 sequences. Skewed Design refers to the theoretical distribution attainable using skewed oligonucleotides. Skewed Sequence refers to the distribution attained experimentally using skewed nucleotides.

FIG. 3 is a plot depicting library source probability. For each binding clone sequence, the probability of origination from each library was calculated based on library design. The relative preferences for G4 versus NNB (o) or G4 versus YS (x) are presented for each loop as well as the total domain. Each symbol indicates a sequenced clone.

FIG. 4 illustrates the results of a binding competition performed with the indicated Fn clones, the antibody 225, and EGF for the EGFR expressed on A431 cells.

FIGS. 5(A), 5(B), and 5(C) are a series of schematics and graphical results related to EGFR downregulation. Panel (A) shows an Fn3-Fn3 heterobivalent protein with the wild-type FN3 structure from PDB ID 1TTG and a flexible linker drawn approximately to scale (in cartoon form). Panel (B) is a representation of surface EGFR expression. Panel (C) is a bar graph depicting data from the expression study shown in Panel (B) for select constructs with A431 cells. Error bars indicate standard deviation of triplicate samples.

FIG. 6 is a series of sequences including a portion of the pETh-Fn3-Fn3 vector. This construct is used for bacterial expression of Fn3-Fn3 bivalent domains with a C-terminal His6 tag. The Fn3 sequences shown in this vector construct can be replaced by any other genetically modified Fn3 domain, including clones A, B, C, D, and E. The nucleic acid sequence is shown as SEQ ID NO:______, and the amino acid sequence, translated from the ATG in NdeI site onward, is shown as SEQ ID NO: ______. FIG. 6 also includes nucleic acid and protein sequences for Fn3 domains engineered for binding to the indicated target. Sequence data is provided from NheI to BamHI in both the nucleotide and amino acid formats. The engineered binders are designated as clones A-E, FG5, and U5.

FIG. 7 is a bar graph illustrating the results of receptor downregulation studies in various cell lines (HT29, U87, HeLa, HMEC, CHO, and A431) with PBSA as a control, EGF, and the constructs D-C, D-B, and D-E. Values and error bars indicate the mean and standard deviation of triplicate samples. Parenthetical notations (e.g., (0.11M)) indicate the number of EGFR per cell in million (M).

FIG. 8 is a schematic depicting the results of a global phorphorylation analysis. The top portion (above the bold line) represents the fifteen highest responders to EGF treatment, and the bottom portion represents the fifteen highest responders to heterobivalent treatment.

FIG. 9 is a bar graph depicting the results of a study of relative viability of hMEC cells treated with the proteins and constructs indicated for 48 or 96 hours. Column and error bars represent mean and standard deviation of triplicate samples. * indicates data from a single sample.

FIG. 10 is a diagram showing EGFR downregulation by the Fn3-Fn3 constructs indicated in A431, HeLa, and HT29 cells. The mean of triplicate samples is presented.

FIGS. 11(A) and 11(B) are a pair of bar graphs depicting the results of a study of cellular migration following treatment of the cell types indicated with the proteins indicated. + indicates addition of 225 antibody. * indicates that PBSA “wound” was completely healed, thus measurable migration was limited. Column and error bars represent mean and standard deviation of triplicate samples.

FIG. 12 is a schematic of various engineered proteins comprising a genetically modified Fn domain and an immunoglobulin. The constant regions of the heavy chain are labeled CH1, CH2, and CH3, and the constant region of the light chain is labeled CL. The variable domains of the heavy and light chains are labeled VH and VL, respectively, and the genetically modified Fn3 domain is labeled Fn3. The amino (N) and carboxy (C) termini of the heavy and light chains are also indicated. The immunoglobulins are assembled in vitro in two-to-two complexes of heavy and light chain moieties, linked by three disulfide bonds. In the engineered proteins illustrated, Fn3 is fused to the heavy or light chain at the N or C terminus with a flexible linker and the fusion constructs are named as indicated (HN where the Fn3 domain is fused to the N terminus of the heavy chain; HC where the Fn3 domain is fused to the C terminus of the heavy chain; LN where the Fn3 comain is fused to the N terminus of the light chain; and LC where the Fn3 domain is fused to the C terminus of the light chain).

FIG. 13 is a series of sequences of representing Ab-Fn3 fusions.

FIG. 14 is a line graph depicting the results of a study of multispecific antibody binding kinetics. Closed symbols represent the unconjugated 225 antibody and open symbols represent the Ab-Fn3 fusion HN-D. Nonlinear least squares regression fits are shown for 225 (solid lines) and HN-D (dashed lines) at pH 6.0 (darker solid and dashed lines) and pH 7.4 (lighter solid and dashed lines).

FIG. 15 is a schematic of multispecific antibody-induced clustering. Engineered proteins that are multispecific and bind two non-competitive epitopes on a target receptor may induce linear or circular chains of crosslinked receptor on the cell surface.

FIG. 16 is a series of photomicrographs providing visual evidence of multispecific antibody-induced clustering. Scale bars=30 μm.

FIGS. 17(A) and (B) are schematics representing the extent of EGFR downregulation in the cell types indicated with engineered proteins indicated.

FIG. 18 is a line graph plotting surface EGFR (% untreated) over time following Ab-Fn3 treatment in A431 cells. The lighter line tracks receptor downregulation following treatment with the Ab-Fn3 fusion HN-D, and the darker line tracks receptor downregulation following treatment with the mAb combination 225+H11. First-order kinetic curves were fit using nonlinear least squares regression.

FIGS. 19(A), (B), and (C) are a series of plots demonstrating that EGFR and its downstream effectors are not agonized by combination mAb treatment. In FIG. 19(A), activation profiles are shown for EGF (▪), 225 (o), H11 (□), and 225+H11 (). Phosphoprotein fluorescence was normalized by DNA fluorescence, and signal relative to that of untreated cells is plotted versus time (±SD; n=3). In FIG. 19(B), normalized phosphoprotein signal is plotted for cells treated with EGF (▪), 225 (o), H11 (□), 225+H11 (), and an antibody-free control ( [symbol] ) (±SD; n=3). In FIG. 19(C), serum-starved A431 cells were incubated with 225, H11, the 225+H11 combination, and EGF at 37° C. for 15 minutes (top) or 60 minutes (bottom).

FIG. 20 is a pair of bar graphs plotting relative cell migration (left-hand graph) and proliferation (right-hand graph) of HMEC (dark gray) and autocrine EGF-secreting ECT (light gray) cells following combination mAb treatment. Relative migration is shown as fractional wound replenishment compared to that of an untreated control ((±SD; n=6). Relative proliferation is presented as viable cell abundance compared to that of untreated cells (±SD; n=6). Asterisks denote P less than 0.01 for the 225+H11 combination relative to either mAb alone.

FIG. 21 is a Table summarizing Fn3 library design. “Pos.” and “WT” are the amino acid position and residue in the human wild-type tenth type III domain. “Access.” is the ratio of solvent accessible surface area for the residue in the fibronectin domain compared to the residue in a random coiled peptide. “Stability” is the relative increase in yeast surface display level of a library with wild-type conservation at the position of interest. “Native” indicates the frequencies of the indicated amino acids in type III fibronectin domains of ten species. “Binders” indicates the enrichment of wild-type (or homolog as indicated) in engineered binders relative to the naïve frequency. “Library Design” indicates the intended amino acid distribution in the new library. “Ab div.” is the designed amino acid distribution that mimics antibody CDR-H3. * indicates the location of loop length variability.

FIG. 22 is a Table summarizing engineered binder sequences. “Name” is the name of each clone. “Target” is the cognate protein bound by the Fn3 clone. “23” refers to the amino acid present at position 23, which is aspartic acid (D) in wild-type Fn3; all positions diversified in the naïve library are likewise presented. “Framework” refers to amino acid mutations outside of the diversified loops. A dash (-) indicates no amino acid.

FIG. 23 is a Table summarizing a stability analysis. The NNB and G4 libraries were independently sorted for clones of low stability and high stability. Sequences of about 50 clones from each sorted population were analyzed. “AA” indicates the wild-type amino acid at positions with wild-type bias or amino acids of elevated frequency at positions without wild-type bias. “G4 Design” indicates the designed frequency of the indicated amino acid. “NNB” and “G4” indicate the difference in amino acid frequency between the high and low stability populations from the indicated library.

FIG. 24 is a Table regarding codon design. The nucleotide mixture used in synthesis at each diversified position is indicated.

FIG. 25 is a Table regarding EGFR binders. “Kd” indicates equilibrium dissociation constant for binding to A431 cells on ice or yeast at 22° C. “nb” indicates no detectable binding. A dash (-) indicates data not collected.

### DETAILED DESCRIPTION

The present invention is based, in part, on our discovery of engineered proteins that include at least one genetically modified Fn domain. Where more than one domain is included, each domain may bind a different epitope on a molecular target, and the two epitopes may be non-overlapping. For example, in one embodiment, the engineered protein includes a first genetically modified Fn domain that specifically binds a first epitope on a molecular target (e.g., a cellular receptor) and a second genetically modified Fn domain that specifically binds a second epitope on the same target or a distinct target. In another embodiment, the engineered protein includes a genetically modified Fn domain that specifically binds a first epitope on a molecular target and a heterologous protein that specifically binds a second epitope on the same target or a distinct target.

We may refer to the “engineered protein(s)” as (a) “binding reagent(s)” and, on occasion these terms may be abbreviated to simply “protein(s)” or “binder(s).” It is to be understood that the engineered proteins of the present invention are not naturally occurring proteins. Accordingly, we may refer to the proteins generally or to a portion thereof (e.g., a Fn domain) as “genetically modified” to indicate that the protein is non-naturally occurring or is a mutant of a wild-type sequence.

As noted above, an engineered protein (or a portion thereof (e.g., a genetically modified Fn domain or target-specific protein scaffold)) may be purified or isolated, in which case it has been substantially separated from materials with which it was previously associated. For example, an engineered protein can be isolated or purified following chemical synthesis or expression in cell culture; the engineered proteins can be separated from the synthesis reagents or the cellular material of the expression system. An isolated or purified engineered protein (or a portion or domain thereof) may be at least or about 75%, 80%, 85%, 90%, 95%, 96%, 97%, 98%, or 99% pure. In the compositions of the invention, the engineered proteins may be present at high concentrations (in which case the compositions may be useful as stock solutions or in in vitro analysis) or at physiologically acceptable concentrations (in which case the compositions would be suitable for administration to a patient).

The Fn Domain:

The Fn domains included in the present proteins can be based on a type III Fn domain (Fn3), such as the tenth type III domain of human fibronectin. This scaffold is small (94 amino acids, ˜10 kDa), stable (7.5-9.4 kcal/mol, Tₘ=90° C.; Cota and Clarke, Protein Sci., 9:112-120, 2000; Parker et al., Protein Engineering Design and Selection, 18:435-444, 2005), soluble to 15 mg/mL, free of cysteines, and expressed at ˜50 mg/L in E. coli (Xu et al., Chemistry & Biology, 9:933-942, 2002). Depending on the degree of modification, it is reasonable to expect low immunogenicity in vivo due to this domain's stability and natural abundance. The Fn3 domain occurs in ˜2% of animal proteins (Bork and Doolittle, Proc. Natl. Acad. Sci. USA, 89:8990-8994, 1992). In addition, both solution (Main et al., Cell, 71:671-688, 1992) and crystal (Dickinson et al., Journal of Molecular Biology, 236:1079-1092, 1994) structures of Fn3 have been determined, thus enabling rational elements of design. The scaffold contains three solvent-exposed loops on either side of parallel β-sheets, somewhat akin to the immunoglobulin fold. Significant evidence shows that Fn3 loops can tolerate diversity to potentially function in a manner analogous to complementarity-determining regions of antibodies. Sequence analyses reveal large variations in the BC and FG loops (Fn3 loops can be referenced by the two peripheral β-strands) with moderate variation in DE loop sequences. NMR spectroscopy indicates significant flexibility of the FG loop as well as moderate flexibility of the BC loop (Can et al., Structure, 5:949-959, 1997). Moreover, elongation by insertion of four glycine residues is moderately well tolerated (1.2, 2.3, and 0.4 kcal/mol destabilization of BC, DE, and FG) (Batori et al., Protein Eng., 15:1015-1020, 2002)). The opposing loops, AB, CD, and EF, offer potential for a bispecific scaffold but are neither as well arranged nor as tolerable of insertion as the other loops. In short, we expect engineered proteins that include genetically modified Fn3 domains may have several biophysical advantages over antibodies, and we consider them an attractive scaffold for use in the proteins described herein.

Naturally occurring Fn3 domains can bind integrins, as the FG loop contains the Arg-Gly-Asp tripeptide (Pierschbacher et al., J. Cell Biochem., 28:115-126, 1985). In the initial use of the domain as a scaffold for molecular recognition, randomization of the BC loop and a shortened FG loop yielded micromolar binders to ubiquitin (Koide et al., The Journal of Molecular Biology, 284:1141-1151, 1998). Thus, although Fn3 could accommodate mutations in loop residues without notable structural change and could acquire novel binding function, a reduced stability, reduced solubility, and non-specific, low affinity binding was also observed. Screening of a library with more extensive randomization of the BC, DE, and FG loops yielded binders to tumor necrosis factor α and vascular endothelial growth factor receptor 2 (VEGF-R2) of nanomolar affinity (Parker et al., Protein Engineering Design and Selection, 18:435-444, 2005; Xu et al., Chemistry & Biology, 9:933-942, 2002). Further maturation produced binders of sub-nanomolar affinity, demonstrating the potential for high affinity binding with Fn3. Engineered Fn3 variants have been used intracellularly (Koide et al., Proc. Natl. Acad. Sci. USA, 99:1253-1258, 2002) as inhibitors in cell culture (Richards et al., Journal of Molecular Biology, 326:1475-1488, 2003), in protein arrays (Xu et al., Chemistry & Biology, 9:933-942, 2002), and as labeling reagents in flow cytometry (Richards et al., Journal of Molecular Biology, 326:1475-1488, 2003) and Western blots (Karatan et al., Chemistry & Biology, 11:835-844, 2004). An anti-VEGF-R2 Fn3 is progressing through clinical trials (and VEGF receptors can be targeted with the present engineered proteins, as described further below).

Where the engineered proteins include two genetically modified Fn domains, the orientation of the domains with respect to one another can be varied. For example, the first and second Fn domains can be arranged in a head-to-tail, head-to-head, or tail-to-tail configuration. This is also true where the engineered proteins include a linker or a heterologous amino acid sequence. For example, the first and second fibronectin domains can be fused, via a linker, in a head-to-tail orientation. Where a heterologous sequence is present, the first and second fibronectin domains can be fused to one another in a head-to-tail configuration (with or without a linker) and fused to the heterologous sequence (with or without a linker). Thus, a linker can be included between the Fn domains and the heterologous sequence, and the Fn domain(s) can be fused to the heterologous sequence at an amino-terminus, carboxy-terminus, or both. The orientation of the genetically modified Fn domain with respect to the heterologous amino acid sequence is discussed further below.

The genetically modified Fn domains used in the engineered proteins of the present invention can be characterized in several ways, including by the extent to which their amino acid sequence is identical to the amino acid sequence of a reference protein. We may refer to this similarity as “percent identity,” and it can be readily determined by comparison of two sequences by eye and simple calculation or by submitting the two sequences (e.g., a modified Fn3 sequence and a reference sequence to a sequence analysis program with the default parameters as defined therein. The reference sequence can be, for example, a corresponding wild-type sequence or a “parent” sequence into which one or more additional mutations were introduced. For example, the reference sequence for a genetically modified tenth Fn3 domain of human fibronectin can be the wild-type tenth Fn3 domain of human fibronectin.

As noted above, where two genetically modified Fn domains are included in an engineered protein, the two domains can be described as having a certain degree of identity as well. In any case, variability can be due to the addition, deletion or substitution of one or more amino acid residues, or to a combination of such changes. Where one residue is substituted for another (e.g., where a wild-type residue is changed), the substituted residue may represent a conservative or non-conservative change. A first genetically modified Fn domain and a second genetically modified Fn domain can be at least or about 40%, 50%, 60%, 70%, 75%, 80%, 85%, 90%, 95%, 98%, or 99% identical. A genetically modified Fn domain and a wild-type Fn domain can be at least or about 40%, 50%, 60%, 70%, 75%, 80%, 85%, 90%, 95%, 98%, or 99% identical. Thus, the engineered proteins of the present invention can include a mutant of the tenth type III fibronectin domain that is at least 40% identical to the corresponding wild-type tenth type III fibronectin domain (e.g., a mammalian (e.g., human) Fn domain).

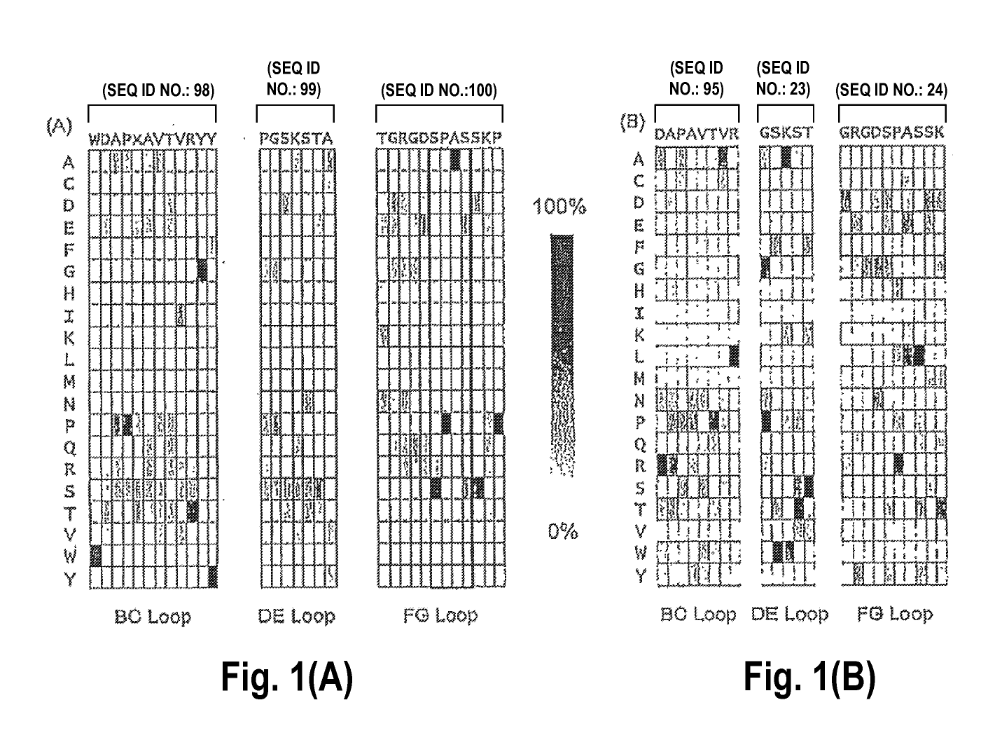

In the Examples presented below, we describe mutational flexibility at a number of positions within an Fn3 domain and within binder sequences. FIGS. 1(A) and 1(B) show the results of this analysis. Various sequences were aligned, and amino acid frequency at each position was evaluated. The results are presented based on an intensity scale; the more frequently a residue appears at a given position in the aligned sequences, the darker the box representing that residue in the plot. To analyze wild-type Fn3 domains, we aligned sequences from chimpanzee, cow, dog, horse, homan, mouse, opossum, platypus, rat, and rhesus monkey. As shown in FIG. 1(A), we analyzed three sequences within the Fn3 domain that encompass the BC, DE, and FG loops. The peripheral residues W22, Y32, P51, A57, and P87 are well conserved while T76 is variable. Accordingly, the genetically modified Fn3 domains used in the present engineered proteins include those in which the wild-type residues corresponding to positions 22, 32, 51, 57, and 87 are not modified (e.g., deleted or replaced) but the residue at position 76 is mutated (e.g., deleted or replaced). Alternatively, amino acid residues that are highly conserved may be substituted conservatively. Other amino acid residues that, based on their conservation, may be retained or conservatively substituted are those at positions A24, P25, V29, G52, S53, S55, G77, G79, and S85. Conversely, the Y at position 31 in the BC loop and the central lysine in the DE loop can be varied more broadly. This conservation data guides protein library and mutant design to improve protein functionality; i.e., proteins with conservation at some of the indicated positions will, on average, possess greater functionality than proteins without conservation.

Our sequence analysis of twenty binders from the G4 library indicates that the desirable biased amino acids (Y, S, G, D, and R) are maintained at high levels in binder sequences whereas undesirable biased amino acids (C and H) are slightly reduced. This supports the hypothesis that Y, S, G, D, and R are indeed favorable whereas C and H are less favorable, which can guide protein library and mutant design.

Another way the genetically modified Fn domains used in the engineered proteins of the present invention can be characterized is by their affinity for the molecular target they were designed to specifically bind. For example, a genetically modified Fn domain (or one of the target-specific protein scaffolds described below) can bind a molecular target with an affinity in the pM to nM range (e.g., an affinity of less than or about 1 pM, 10 pM, 25 pM, 50 pM, 100 pM, 250 pM, 500 pM, 1 nM, 5 nM, 10 nM, 15 nM, 20 nM, 25 nM, 30 nM, 40 nM or 50 nM).

Genetically modified Fn domains can also be classified as having or lacking conformational sensitivity. Such sensitivity is present when the genetically modified Fn domain specifically binds its molecular target in a naturally folded configuration but fails to do so (or does so with a greatly reduced affinity) when the target is denatured.

In addition to these characteristics, any given genetically modified Fn domain (or any given heterologous sequence) can be characterized in terms of its ability to modify cell behavior (e.g., cellular proliferation or migration) or to positively impact a symptom of a disease, disorder, condition, syndrome, or the like, associated with the expression or activity of the molecular target. For example, the genetically modified Fn domain can be one that inhibits the ability of cancerous cells to proliferate or migrate and/or improves a symptom in a patient having a cancer associated with aberrant expression of the molecular target. For example, the EGFR is associated with numerous cancers, and the modified Fn domain included in an engineered protein can be one that specifically binds the EGFR and inhibits cellular proliferation or migration in the bound EGFR-expressing cells. Similarly, the modified Fn domain included in an engineered protein can be one that specifically binds EGFR-expressing cancer cells in a patient and improves a symptom the patient is experiencing or provides some other clinical benefit. In other words, the modified Fn domain and an engineered protein of which it is a part can be used to treat a patient who is suffering from a disease (e.g., cancer) that is associated with aberrant expression of a molecule targeted by the modified Fn domain or engineered protein. While target specificity is a feature of the engineered proteins, we wish to stress that the compositions and methods of the invention are not limited to those that elicit any particular cellular response or work through any particular mechanism of action.

In vitro assays for assessing binding to a molecular target, cellular proliferation, and cellular migration are known in the art. For example, where the molecular target is an EGFR, binding, proliferation, and migration assays can be carried out using A431 epidermoid carcinoma cells, HeLa cervical carcinoma cells, and/or HT29 colorectal carcinoma cells. Other useful cells and cell lines will be known to those of ordinary skill in the art. For example, genetically modified Fn3 domains (and/or engineered proteins containing them) can be analyzed using U87 glioblastoma cells, hMEC cells (human mammary epithelial cells), or Chinese hamster ovary (CHO) cells. The molecular target can be expressed as a fluorescently tagged protein to facilitate analysis of an engineered protein's effect on the target. For example, the assays of the present invention can be carried out using a cell type as described above transfected with a construct expressing an EGFR-green fluorescent protein fusion. An engineered protein may inhibit cellular proliferation or migration by at least or about 30% (e.g., by at least or about 30%, 40%, 50%, 65%, 75%, 85%, 90%, 95% or more) relative to a control (e.g., relative to proliferation or migration in the absence of the engineered protein or a scrambled engineered protein).

Of course, the genetically modified Fn domains may be described as having a combination of the characteristics described above. For example, a genetically modified Fn domain that exhibits a certain percentage of sequence identity to a reference sequence can also be a domain that exhibits an affinity for the target molecule in the pM to nM range and/or exhibits conformational sensitivity. Similarly, the genetically modified Fn domain can be at least or about 40%, 50%, 60%, 70%, 75%, 80%, 85%, 90%, 95%, 98%, or 99% identical to a reference sequence (e.g., the naturally occurring domain from which it was derived) and can inhibit the proliferation or migration of a cell expressing a molecular target to which the modified Fn domain specifically binds.

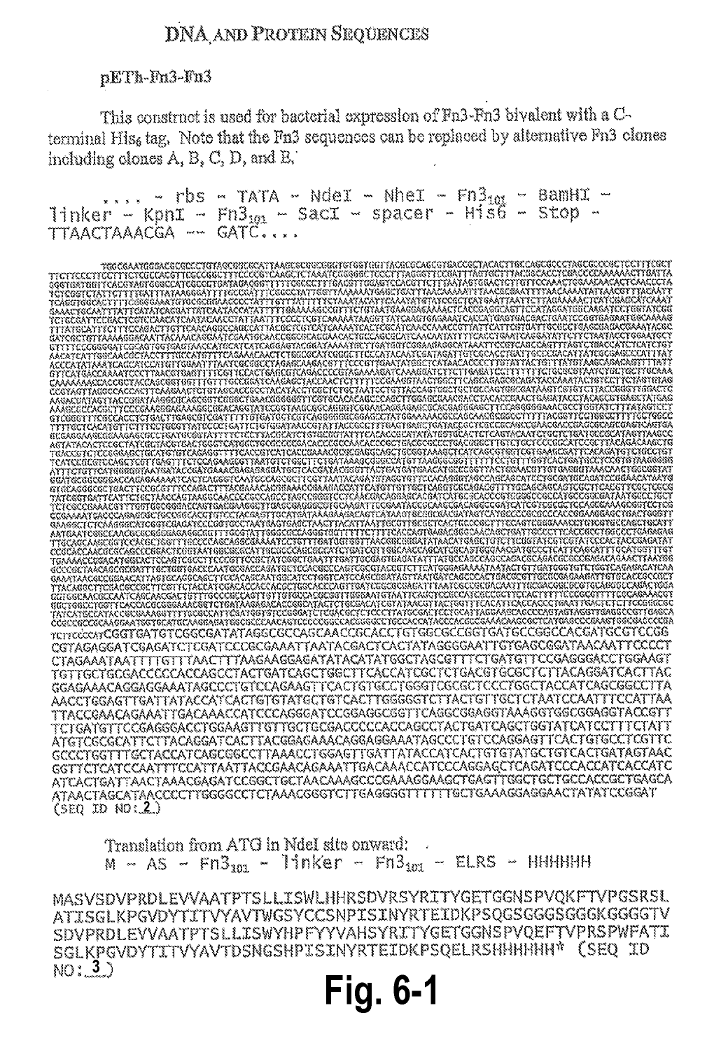

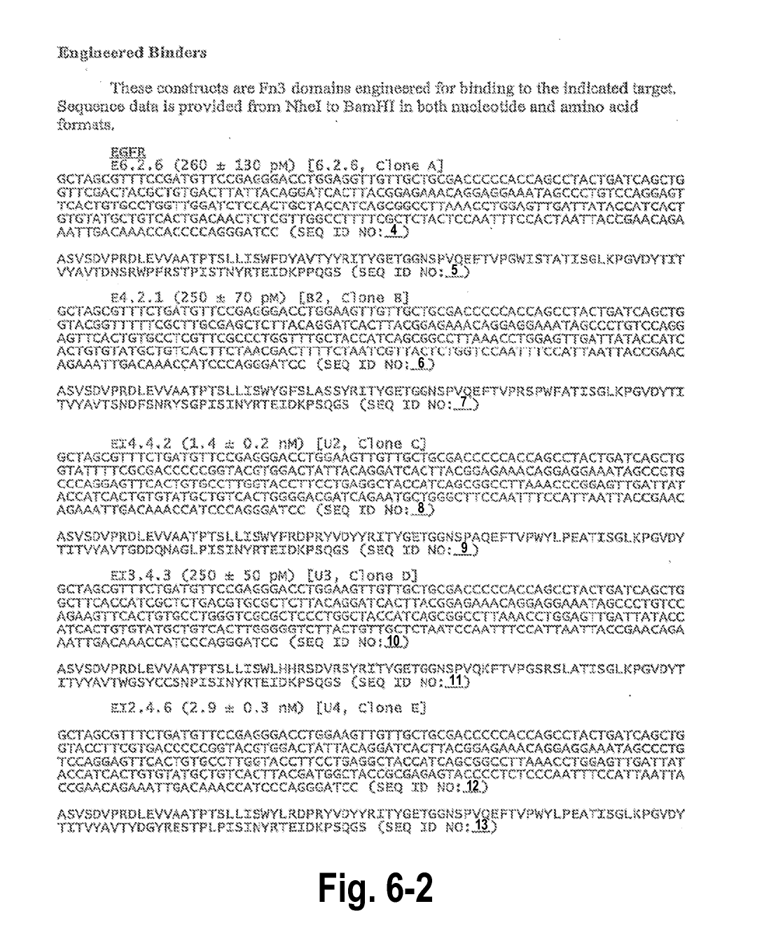

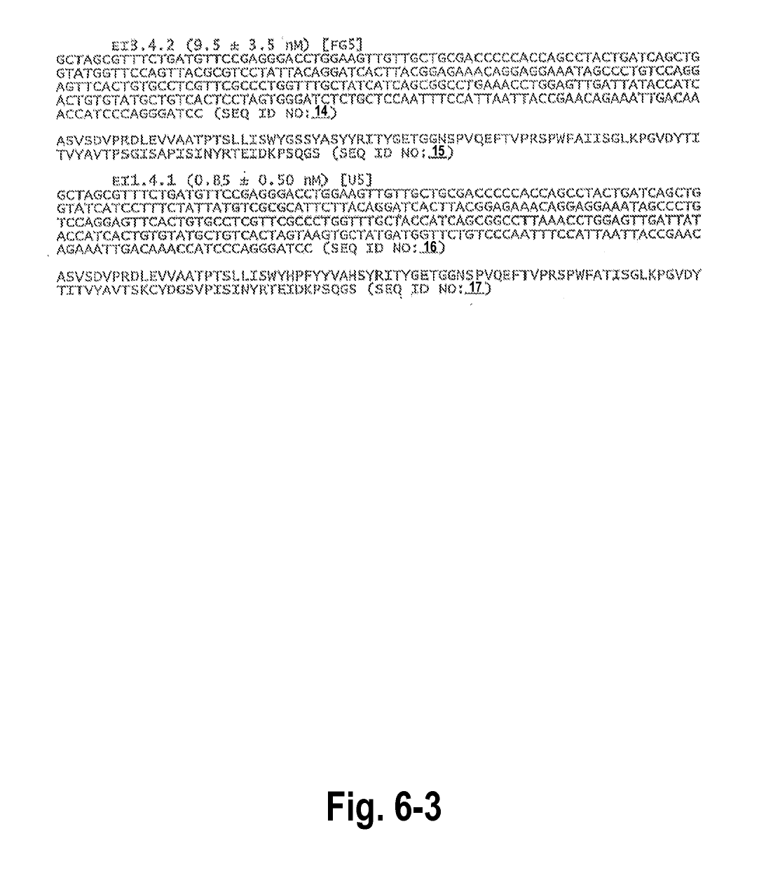

More specifically, a genetically modified Fn domain can have or can include the amino acid sequence of a Fn3 domain described herein as clone A, clone B, clone C, clone D, or clone E (see FIG. 6). Further, an engineered protein can be or can include a pair of these clones, which may be fused to one another via a linker. For example, the engineered proteins can include a pair of genetically modified Fn domains that have or that include the sequence of clone A, clone B, clone C, clone D, or clone E. Useful bivalents for targeting and downregulating an EGFR include D-B, D-C, D-D, D-E, A-D, B-D, C-D, and E-D. The domains may be linked in the order indicated. As noted, genetically modified Fn domains, including the bivalents described here, can be fused, directly or via a linker, to a heterologous amino acid sequence such as an immunoglobulin. The amino terminal, carboxy terminal, or both, of either the heavy or light chain (e.g., in an IgG) can serve as the point of attachment, and specfic configurations are discussed further below.

Heterologous Amino Acid Sequences:

The engineered proteins of the invention can include, in addition to a genetically modified Fn domain: (a) a target-specific protein scaffold, and/or (b) an accessory amino acid sequence.

The affinity of the target-specific protein scaffold for its target may be increased when the scaffold is joined to one or more genetically modified fibronectin domains (as described herein). For example, the affinity of an antibody for its molecular target may be at least or about an order of magnitude greater than the affinity of the antibody alone at either endosomal pH (6.0), physiological pH (7.4), or both.

The target-specific protein scaffold can be an immunoglobulin (e.g., an IgG or a biologically active (e.g., antigen-binding) portion or variant thereof (e.g., an scFv)), a designed ankyrin repeat protein, an anticalin, or an affibody. These scaffolds for molecular recognition are known in the art, as are residues that are generally diversified to generate novel binding function. Accordingly, where the engineered proteins include a heterologous amino acid sequence, that sequence can be (or can be derived from; a mutant of) an ankyrin repeat protein, an anticalin, an affibody, or an immunoglobulin, including a fragment or other variant thereof (e.g., an scFv). One can use information regarding generally diversified residues to select residues for diversification to generate protein binders to the targets described herein. One can also subject these protein scaffolds to directed evolution as described herein for Fn domains in order to generate binders with improved specificity and affinity for a given molecular target.

We may use the term “immunoglobulin” synonymously with “antibody.” An immunoglobulin can be a tetramer (e.g., an antibody having two heavy chains and two light chains) or a single-chain immunoglobulin. Further, the immunoglobulin may be an intact immunoglobulin of type IgA, IgG, IgE, IgD, IgM (as well as subtypes thereof (e.g., IgG₁, IgG₂, IgG₃, and IgG₄)).

Examples of antigen-binding portions or fragments or other immunoglobulin variants that can be used in the present proteins include: (i) an Fab fragment, a monovalent fragment consisting of the VLC, VHC, CL and CH1 domains; (ii) a F(ab′)₂ fragment, a bivalent fragment comprising two Fab fragments linked by a disulfide bridge at the hinge region; (iii) a Fd fragment consisting of the VHC and CH1 domains; (iv) a Fv fragment consisting of the VLC and VHC domains of a single arm of an antibody, (v) a dAb fragment (Ward et al., Nature 341:544-546, 1989), which consists of a VHC domain; and (vi) an isolated complementarity determining region (CDR) having sufficient framework to specifically bind, e.g., an antigen binding portion of a variable region. An antigen-binding portion of a light chain variable region and an antigen binding portion of a heavy chain variable region, e.g., the two domains of the Fv fragment, VLC and VHC, can be joined, using recombinant methods, by a synthetic linker that enables them to be made as a single protein chain in which the VLC and VHC regions pair to form monovalent molecules (known as single chain Fv (scFv); see e.g., Bird et al., Science 242:423-426, 1988; and Huston et al., Proc. Natl. Acad. Sci. USA 85:5879-5883, 1988). Such single chain antibodies are also intended to be encompassed within the term “antigen-binding portion” of an antibody or as “a variant” of an antibody.

These antibody portions or fragments are obtained using conventional techniques known to those of ordinary skill in the art, and the portions are screened for utility in the same manner as are intact antibodies. An Fab fragment can result from cleavage of a tetrameric antibody with papain; Fab′ and F(ab′)2 fragments can be generated by cleavage with pepsin.

In summary, single chain immunoglobulins, and chimeric, humanized or CDR-grafted immunoglobulins, including those having polypeptides derived from different species, can be incorporated into the engineered proteins.

The various portions of these immunoglobulins can be joined together chemically by conventional techniques, or can be prepared as contiguous polypeptides using genetic engineering techniques. For example, nucleic acids encoding a chimeric or humanized chain can be expressed to produce a contiguous polypeptide. See, e.g., Cabilly et al., U.S. Pat. No. 4,816,567; Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; and Winter, European Patent No. 0,239,400 B1. See also, Newman et al., BioTechnology, 10:1455-1460, 1992, regarding CDR-graft antibody, and Ladner et al., U.S. Pat. No. 4,946,778 and Bird, R. E. et al., Science 242:423-426, 1988 regarding single chain antibodies.

Accessory Sequences:

The accessory sequence can be one that prolongs the circulating half-life of the genetically modified Fn domain or an engineered protein of which it is a part, a polypeptide that facilitates isolation or purification of the engineered protein, an amino acid sequence that facilitates the bond (e.g., fusion or conjugation) between one part of the engineered protein and another or between the engineered protein and another moiety (e.g., a therapeutic compound), an amino acid sequence that serves as a label, marker, or tag (including imaging agents), or an amino acid sequence that is toxic.

The amino acid sequence that increases the circulating half-life can be an Fc region of an immunoglobulin, including an immunoglobulin that has a reduced binding affinity for an Fc receptor (such as those described in U.S. Patent Application No. 20090088561, the content of which is hereby incorporated by reference in its entirety). As the engineered proteins of the present invention can include immunoglobulin sequences, and as the Fc region can increase circulating half-life, where the engineered proteins include an immunoglobulin as the heterologous, target-specific protein scaffold, the Fc region of the immunoglobulin can also serve to increase the protein's circulating half-life; the accessory sequence can be a part of the heterologous amino acid sequence.

Half-life can also be increased by the inclusion of an albumin (or a portion or other variant thereof that is large enough to have a desired effect on half-life). The albumin can be a serum albumin, such as a human or bovine serum albumin.

The Fn domain or another portion of the engineered protein can also be “pegylated” using standard procedures with poly(ethylene glycol). Engineered proteins that are pegylated may have an improved circulating half-life.

Where the engineered protein includes an accessory protein that facilitates isolation or purification, that protein can be a tag sequence designed to facilitate subsequent manipulations of the expressed nucleic acid sequence (e.g., purification or localization). Tag sequences, such as green fluorescent protein (GFP), glutathione S-transferase (GST), c-myc, hemagglutinin, β galactosidase, or Flag™ tag (Kodak) sequences are typically expressed as a fusion with the polypeptide encoded by the nucleic acid sequence. Such tags can be inserted in a nucleic acid sequence such that they are expressed anywhere along an encoded polypeptide including, for example, at either the carboxyl or amino termini. The type and combination of regulatory and tag sequences can vary with each particular host, cloning or expression system, and desired outcome.

As noted, the engineered proteins can include linkers at various positions (e.g., between two genetically modified Fn domains or between a genetically modified Fn domain and a heterologous amino acid sequence). The linker can be an amino acid sequence that is joined by standard peptide bonds to the engineered protein. The length of the linker can vary including an essentially absent linker in which the proteins are directly fused and, where it is an amino acid sequence, can be at least three and up to about 300 amino acids long (e.g., about 4, 8, 12, 15, 20, 25, 50, 75, 80, 85, 90, 95, 100, 125, 150, 175, 200, 250 or 300 amino acids long). Moreover, a non-peptide linker such as polyethylene glycol or an alternative polymer could be used. As with all other domains in the engineered proteins, the amino acid residues of the linker may be naturally occurring or non-naturally occurring. We have used a polypeptide linker having the sequence GSGGGSGGGKGGGGT (SEQ ID NO:______), and linkers comprising this sequence or functional variants thereof can be incorporated in the engineered proteins of the present invention. The linkers can be glycine-rich (e.g., more than 50% of the residues in the linker can be glycine residues).

The amino acid sequence that serves as a label, marker, or tag can be essentially any detectable protein. It may be detectable by virtue of an intrinsic property, such as fluorescence, or because it mediates an enzymatic reaction that gives rise to a detectable product. The detectable protein may be one that is recognized by an antibody or other binding protein.

The engineered proteins can also be configured to carry imaging or contrast agents, many of which are known in the art and can be connected to an engineered protein using standard techniques.

Regarding the overall configuration of the engineered proteins that include one or more genetically modified Fn domains and a heterologous amino acid sequence, there can be considerable variation. Multiple genetically modified Fn domains can be included in the engineered proteins. For example, the proteins can include 1-16 (e.g., 1, 2, 4, 8, 12, or 16) genetically modified Fn domains. As noted, the Fn domains can be identical to one another or distinct; they can bind the same, similar, or distinct epitopes, including non-overlapping epitopes; and they can be joined to the amino terminus, the carboxy terminus, or both termini of the target-specific protein scaffold. At a given terminus, one can include a single genetically modified Fn domain or a pair of these domains. Where the protein scaffold is an IgG, one or more genetically modified Fn domains can be joined (e.g., fused) to the amino or carboxy terminus of a light chain (or chains), to the amino or carboxy terminal of a heavy chain (or chains), or to any combination of these positions. In a specific embodiment, the engineered protein can include, as a heterologous sequence, an immunoglobulin (e.g., an IgG) and multiple genetically modified fibronectin domains fused, directly or via a linker, to the amino terminus of a heavy chain or an amino terminus of a light chain of the immunoglobulin. In another embodiment, the engineered protein can include, as a heterologous sequence, an immunoglobulin (e.g., an IgG) and one or more genetically modified fibronectin domains fused, directly or via a linker, to the amino terminus of a heavy chain and one or more genetically modified fibronectin domains fused, directly or via a linker, to the carboxy terminus of the heavy chain of the immunoglobulin. In another embodiment, the engineered protein can include, as a heterologous sequence, an immunoglobulin (e.g., an IgG) and one or more genetically modified fibronectin domains fused, directly or via a linker, to the amino terminus of a light chain and one or more genetically modified fibronectin domains fused, directly or via a linker, to the carboxy terminus of the light chain of the immunoglobulin. In another embodiment, the engineered protein can include, as a heterologous sequence, an immunoglobulin (e.g., an IgG) and one or more genetically modified fibronectin domains fused, directly or via a linker, to either the amino or carboxy terminus or to both termini of a heavy chain and one or more genetically modified fibronectin domains fused, directly or via a linker, to either the amino or carboxy terminus or to both termini of the light chain of the immunoglobulin.

Protein Engineering:

Screening and evolution of combinatorial libraries, using methods both known in the art and described in the Examples below, provides an effective way to generate binding proteins that can be used in the engineered proteins of the invention. The process can be described as involving three key elements: naïve library design, selection of functional clones, and sequence diversification of lead clones. Accordingly, the present invention features methods of generating an engineered protein (or a domain thereof) by directed evolution. The steps of such methods can include providing a naïve combinatorial library of protein clones, selecting or screening the library to identify lead clones, and diversifying the identified clones (e.g., by mutagenesis or informed library synthesis) to produce a next generation library. The cycle of selection and diversification can be repeated (e.g., two to ten times) until the desired functionality (e.g., selective binding to an identified target) is achieved.

Clones with the desired functionality can be identified from the library of protein variants with high throughput selection via linkage of genotype and phenotype. Though this linkage can be achieved through a multitude of display formats (Hoogenboom, Nat. Biotechnol, 23:1105-1116, 2005) such as phage display and mRNA display, yeast surface display is preferred (Hackel and Wittrup, In Protein Engineering Handbook (Bronscheuer, Ed.), Vol. 1. Wiley-VCH, 2009). In vitro technologies tout high theoretical library sizes because of the absence of cellular transformation, which can limit library size. Yet in a recent comparison of yeast surface display and phage display using the same antibody, DNA library, and target antigen, yeast surface display identified three times more clones than did phage display and did not miss a single phage clone revealing that constructed size and functional size can differ substantially (Bowley et al., Protein Engineering Design and Selection, 20:81-90, 2007). Yeast surface display may also enable selection of stable clones because of the quality control apparatus of the eukaryotic secretory system (Shusta et al., Journal of Molecular Biology, 292:949-956, 1999). Fluorescence-activated cell sorting of yeast allows quantitative discrimination of clone functionality (VanAntwerp and Wittrup, Biotechnol, Prog., 16:31-37, 2000).

In yeast surface display, tens of thousands of copies of Fn3 are tethered to the exterior of an individual Saccharomyces cerevisiae yeast cell while the genetic information for the Fn3 clone is maintained in the cell interior. The cell-protein linkage begins with the Aga1p subunit of α-agglutinin, which anchors in the cell wall periphery via β-glucan covalent linkage (Lu et al, J. Cell. Biol., 128:333-340, 1995). The Aga2p subunit, secreted from the yeast cell as a fusion to Fn3, attaches to Agalp via two disulfide bonds. The peptide bond in the fusion protein thus completes the linkage resulting in “display” of Fn3 on the yeast cell. Aga2p and Fn3, linked by a (G₄S)₃ peptide, are followed by HA and c-myc epitopes, respectively, to enable analysis of the display of Aga2p and the full-length protein fusion. Display is achieved through transformation of DNA encoding for the Aga2p-Fn3 fusion followed by cell growth and induction of both Aga1p and Aga2p-Fn3 protein expression using a galactose-inducible GAL promoter. The displayed clones can be screened for their ability to bind to a target of interest, including any of those described herein, using flow cytometry or captured by immobilized antigen.

Selected clones can be evolved through partial diversification of their sequence followed by selection for mutants that exhibit improved functionality. Error-prone PCR to introduce random mutations throughout the gene is the most common method of diversification. Yeast surface display also enables gene shuffling via homologous recombination (Swers et al., Nucleic Acids Research, 32:e36, 2004).

Once identified, whether through phage display, mRNA display, yeast surface display, or by any other mechanism, a protein can be incorporated into the engineered proteins described herein using standard recombinant techniques. These techniques are well known in the art and are discussed further below.

Targets:

A wide variety of molecular targets can be specifically bound and these include molecules expressed on the cell surface, such as receptors for growth factors, neurotransmitters, and the like. The receptor can be a tyrosine kinase receptor, and much of the work with the constructs described in the Examples has focused on the epidermal growth factor (EGF) receptor (EGFR). This receptor is a receptor tyrosine kinase in the ErbB family that comprises three regions: an extracellular region, a transmembrane domain, and an intracellular region that includes a juxtamembrane domain, kinase domain, and a C-terminal tail containing phosphorylation sites. These domains and sites are understood in the art. The extracellular region consists of four domains of which domains I and III are leucine rich repeat folds and domains II and IV are cysteine-rich domains. The receptor is predominantly present in a tethered conformation on the cell surface. Binding of ligand, including epidermal growth factor, transforming growth factor α, epiregulin, amphiregulin, β-cellulin, and heparin-binding epidermal growth factor, stabilizes an open conformation of the receptor. Resultant dimerization enables kinase activation and phosphorylation of the intracellular domain. Phosphorylation sites enable docking of adaptor proteins that initiate signaling cascades such as the mitogen-activated protein kinase pathway activated by Ras and Shc, the Akt pathway activated by phosphatidylinositol-3-OH kinase, and the protein kinase C pathway activated by phospholipase Cγ. These pathways form a complex signaling network that impacts multiple cellular processes including differentiation, migration, and growth (Yarden and Sliwkowski, Nat. Rev. Mol. Cell. Biol., 2:127-137, 2001). Activated EGFR is endocytosed within several minutes and a fraction undergoes fast recycling from the early endosome. The alternate fraction persists to the late endosome resulting in slower recycling or degradation (Sorkin and Goh, Experimental Cell Research., 315:683-696, 2009).

Dysregulation of EGFR-mediated signalling is observed in breast, bladder, head and neck, and non-small cell lung cancers (Yarden and Sliwkowski, Nat. Rev. Mol. Cell. Biol., 2:127-137, 2001). Accordingly, engineered proteins that target the EGFR can be used to treat these cancers.

An analysis of 15 years of published literature on EGFR expression and cancer prognosis revealed that receptor overexpression is associated with reduced survival in 70% of head and neck, ovarian, cervical, bladder, and esophageal cancers (Nicholson et al., Eur. J. Cancer, 37 Suppl. 4, S9-15, 2001). Autocrine production of transforming growth factor α and epidermal growth factor (EGF) correlate with reduced survival in lung cancer (Tateishi et al., Cancer Research, 50:7077-7080, 1990). Receptor mutation is also implicated in cancer. EGFRvIII, which lacks amino acids 6-273, is observed in glioblastoma, non-small cell lung cancer, and cancers of the breast and ovary (Pedersen et al., Ann. Oncol., 12:745-760, 2001). This mutant is unable to bind ligand yet is constitutively active, posing a unique therapeutic challenge, particularly for ligand blocking agents. Ectodomain point mutants in glioblastoma yield tumorigenicity (Lee et al., PLoS. Med., 3:e485, 2006). Kinase domain mutations observed in non-small cell lung cancer hyperactivate kinase (Sharma et al., Nat. Rev. Cancer, 7:169-181, 2007).

As a result of the involvement of EGFR in cancer, there has been substantial effort spent developing receptor inhibitors as therapeutics. The U.S. Food and Drug Administration has approved two monoclonal antibodies and two tyrosine kinase inhibitors targeting EGFR. Cetuximab (Erbitux, Bristol-Myers Squibb), approved for colorectal and head and neck cancer, and panitumumab (Vectibix, Amgen), approved for colorectal cancer, are antibodies that compete with EGF for receptor binding. However, the relative impact of ligand competition, receptor downregulation, and antibody-dependent cellular cytotoxicity is unknown (note that panitumumab is an immunoglobulin G (IgG) 2a molecule and thus incapable of triggering cellular cytotoxicity). Both antibodies exhibit modest efficacy. In treatment of metastatic colorectal cancer refractory to irinotecan tyrosine kinase inhibitor, only 11% of patients respond to cetuximab alone and only 23% respond to cetuximab and irinotecan in combination (Cunningham et al., N. Engl. J. Med., 351:337-345, 2004). In the treatment of head and neck cancer, the addition of cetuximab to radiation extends median survival from 29 to 49 months yet only increases responsiveness from 45% to 55% and improvement is only evident for oropharyngeal cancer but not hypopharyngeal or laryngeal cancers. Moreover, metastases were present at comparable amounts with and without antibody (Bonner et al., N. Engl. J. Med., 354:567-578, 2006). In metastatic colorectal cancer, panitumumab extends progression-free survival from 64 days to 90 days; yet the overall response rate was only 8% and there was no improvement in overall survival (Messersmith and Hidalgo, Clinical Cancer Research, 13:664-4666, 2007).

While this efficacy validates EGFR as a useful therapeutic target, it begs the search for improved understanding of receptor biology and the development of improved therapy. Potential causes of the modest efficacy include inability to effectively compete with ligand, especially in the presence of autocrine signaling; insufficient downregulation of receptor; lack of inhibition of constitutively active EGFRvIII; and mutational escape. Thus, novel binders capable of downregulation and/or inhibition via different modes of action would be beneficial. Small, monovalent binders would enable improved biophysical studies via specific inhibition or Forster resonance energy transfer. Such small binders could also be useful for in vivo imaging to study receptor localization and trafficking.

Other Cancer-Specific Targets:

In addition to the EGFR (e.g., a human EGFR) as a cancer target, the binding reagents can be directed to A33 (e.g., human A33 or mouse A33), and mouse CD276.

Other Cancer-Specific or Receptor Tyrosine Kinase-Specific Targets:

Other targets include receptors of the ErbB, insulin, PDGF, FGF, VEGF, HGF, Trk, Eph, AXL, LTK, TIE, ROR, DDR, RET, KLG, RYK, and MuSK receptor families. For example, the engineered proteins described herein that target a VEGF receptor (e.g., VEGF-R2) can be used in the treatment of multiple myeloma.

Immunological Targets:

Immunological targets include the Fcγ receptors IIa and IIIa, and biotechnological targets include mouse IgG and human serum albumin (HSA).

Biotechnological Targets:

In addition, binders to lysozyme, carcinoembryonic antigen, goat IgG, and rabbit IgG were engineered during platform development.

Nucleic Acids, Vector Constructs, and Expression Systems:

Nucleic acid (e.g., DNA) sequences coding for any of the polypeptides within the present engineered proteins are also within the scope of the present invention as are methods of making the engineered proteins. For example, variable regions can be constructed using PCR mutagenesis methods to alter DNA sequences encoding an immunoglobulin chain, e.g., using methods employed to generate humanized immunoglobulins (see e.g., Kanunan, et al., Nucl. Acids Res. 17:5404, 1989; Sato, et al., Cancer Research 53:851-856, 1993; Daugherty, et al., Nucleic Acids Res. 19(9):2471-2476, 1991; and Lewis and Crowe, Gene 101:297-302, 1991). Using these or other suitable methods, variants can also be readily produced. In one embodiment, cloned variable regions can be mutagenized, and sequences encoding variants with the desired specificity can be selected (e.g., from a phage library; see e.g., Krebber et al., U.S. Pat. No. 5,514,548; Hoogenboom et al., WO 93/06213, published Apr. 1, 1993)).

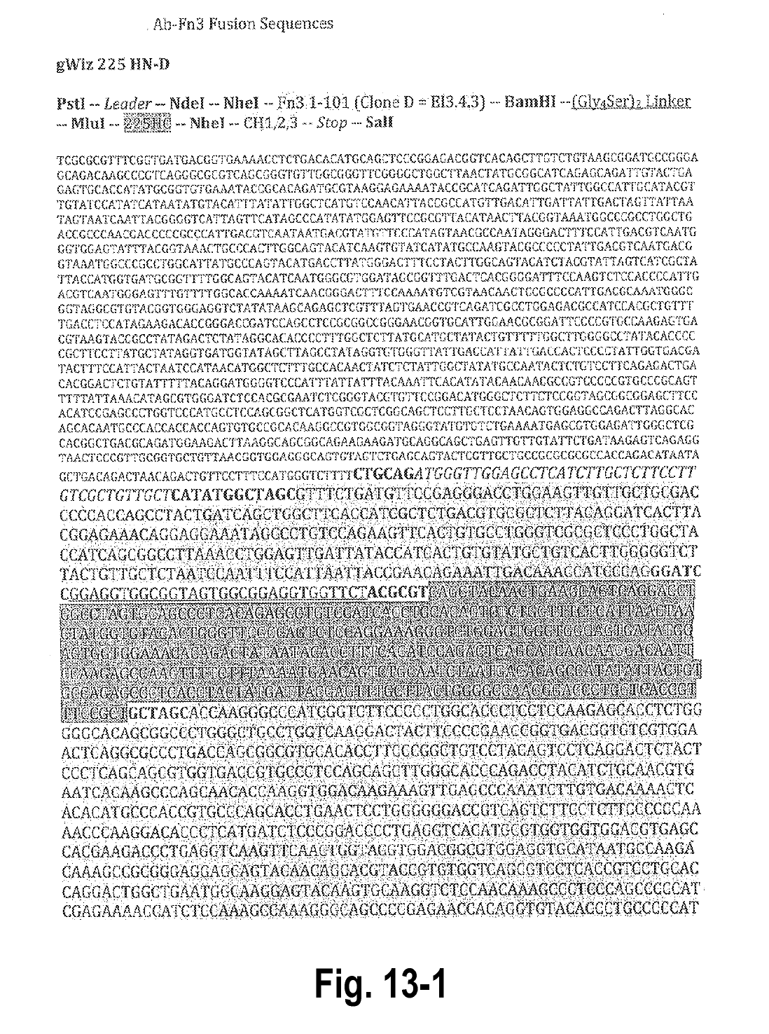

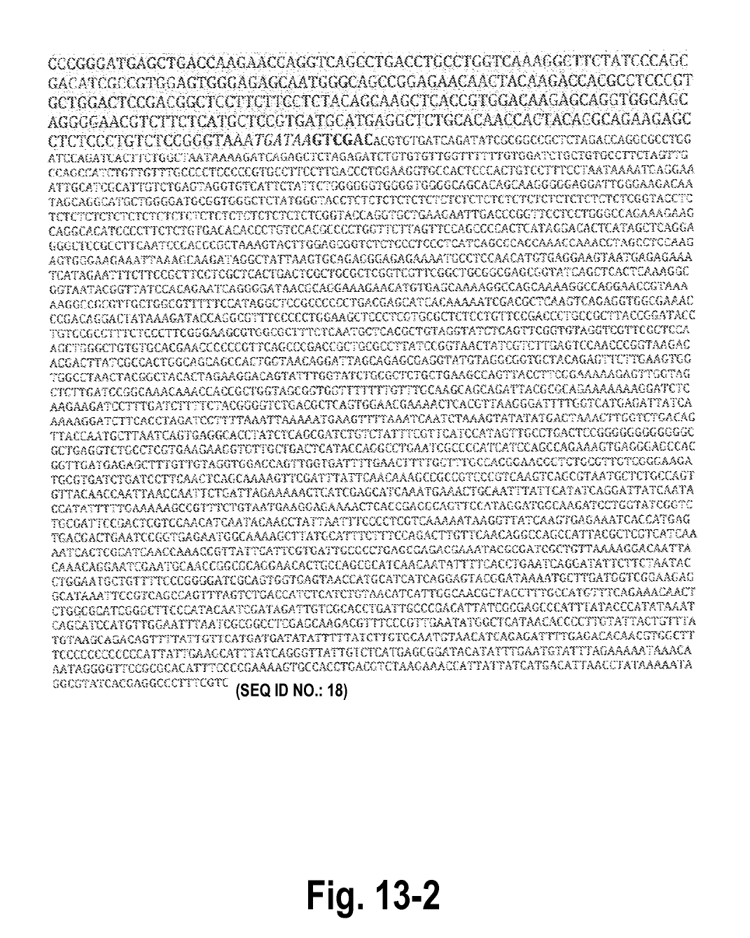

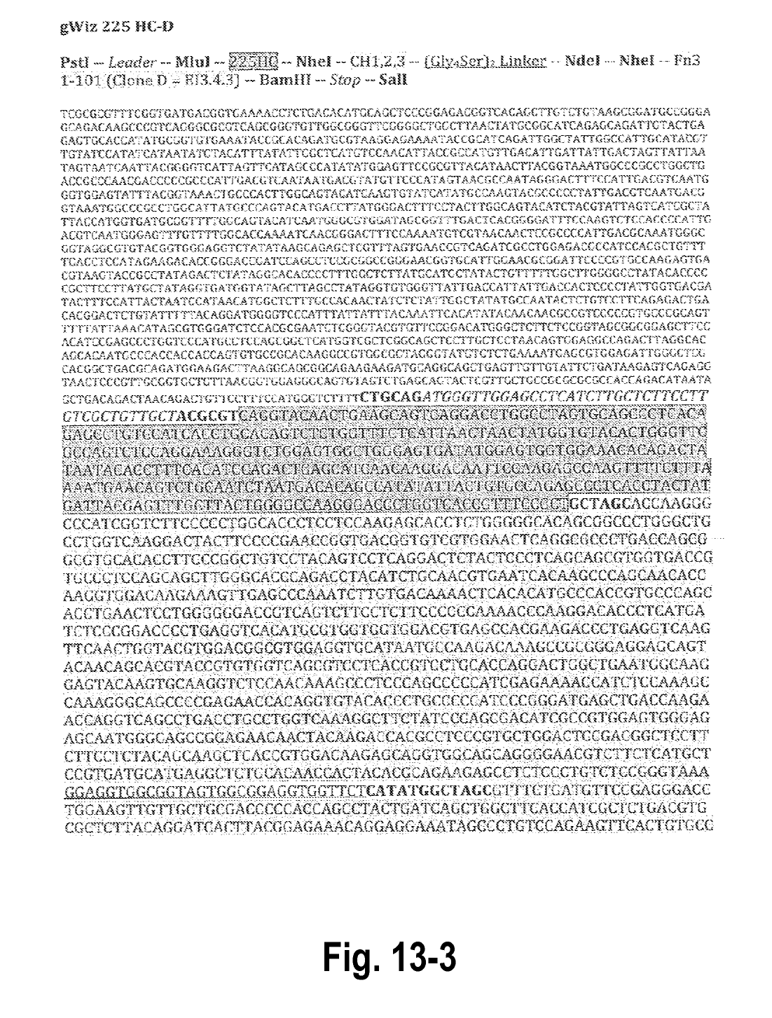

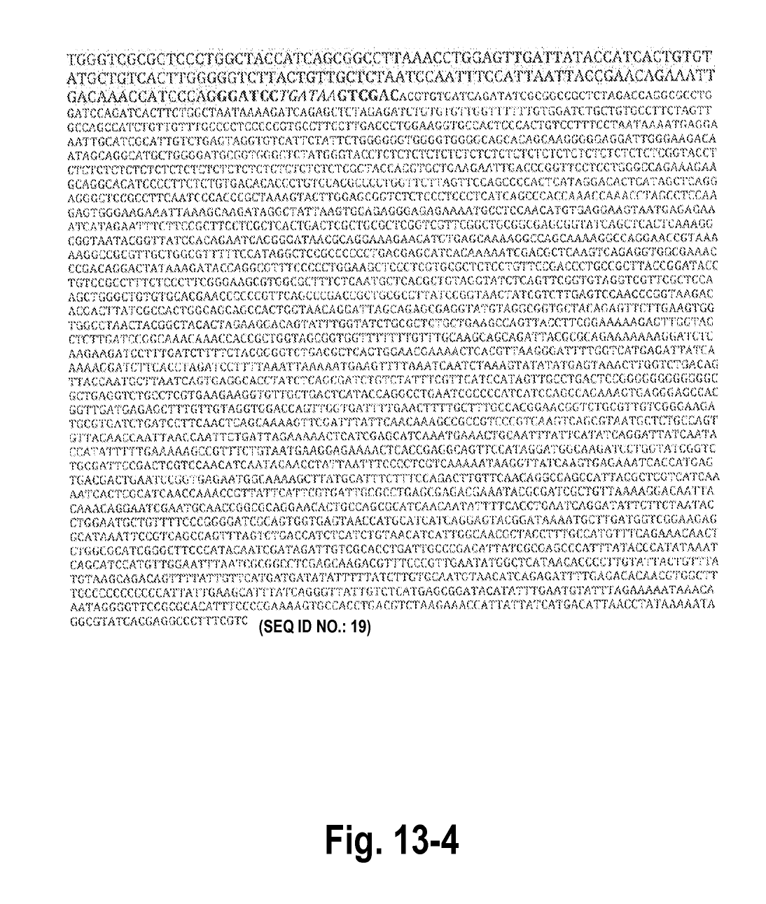

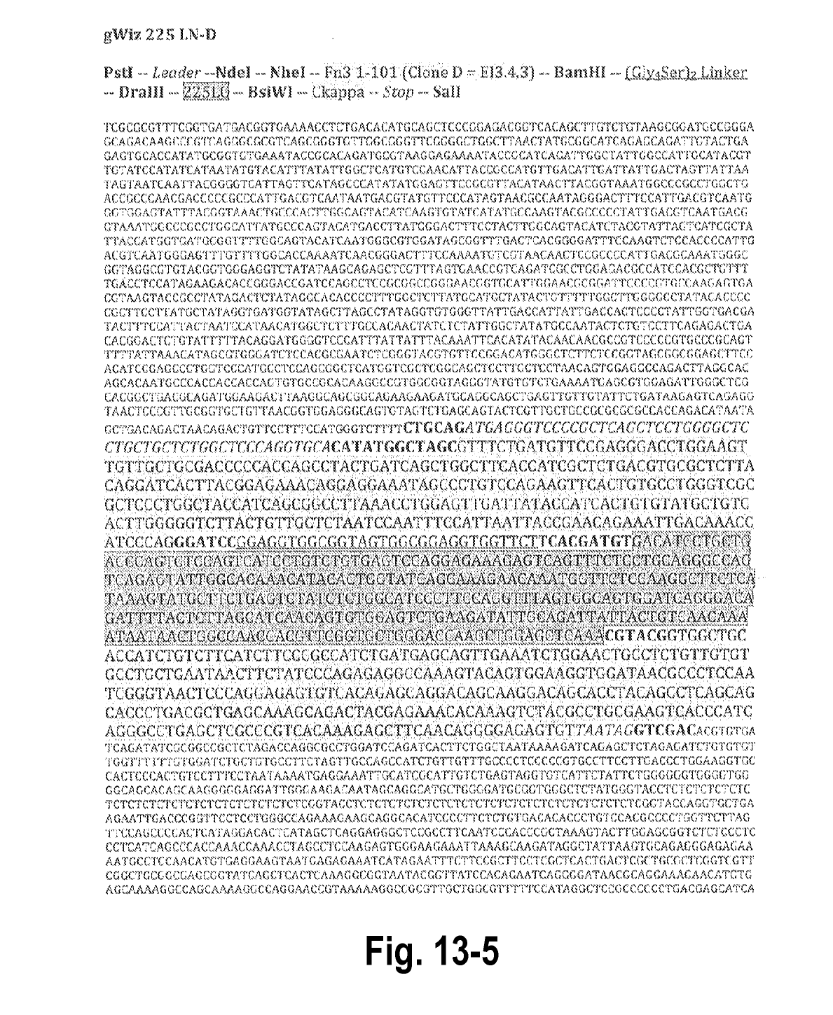

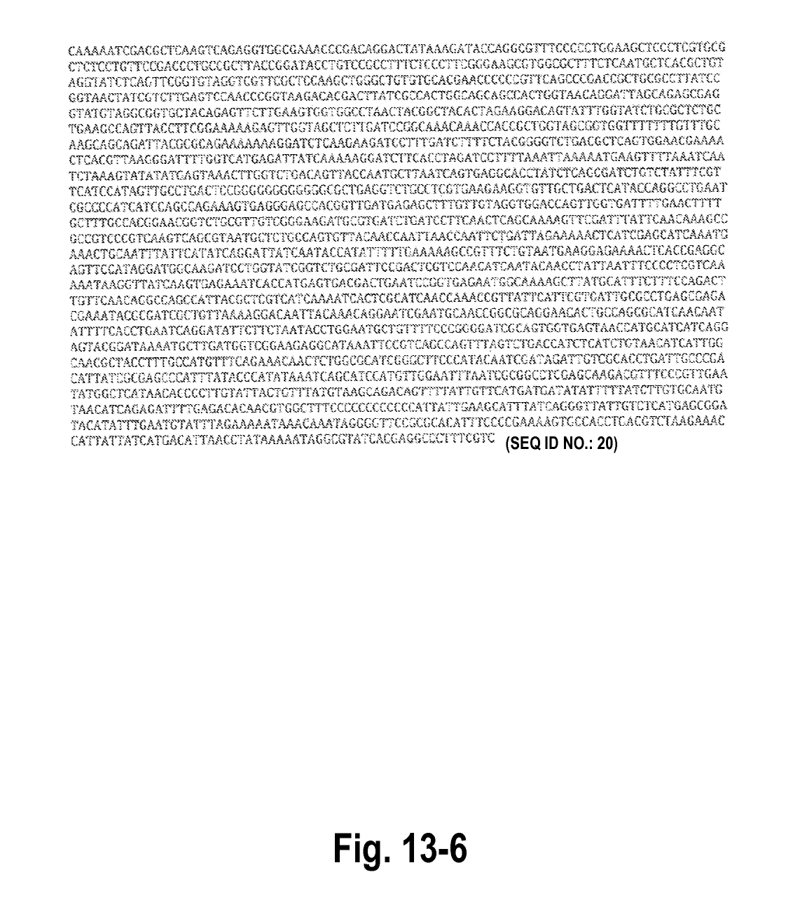

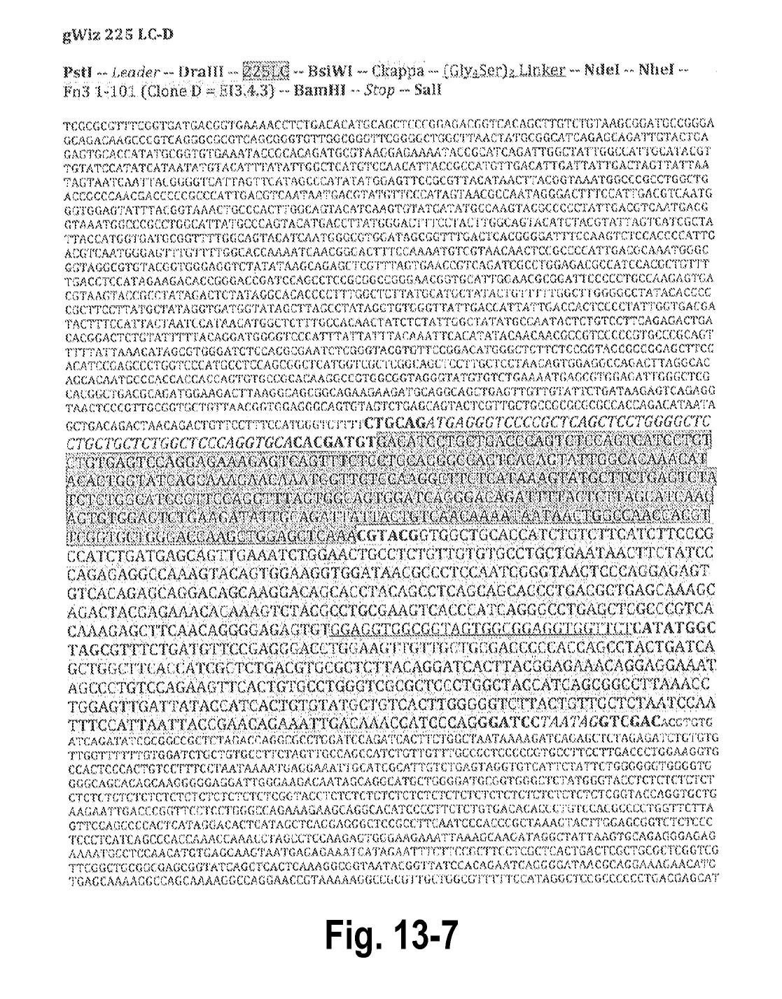

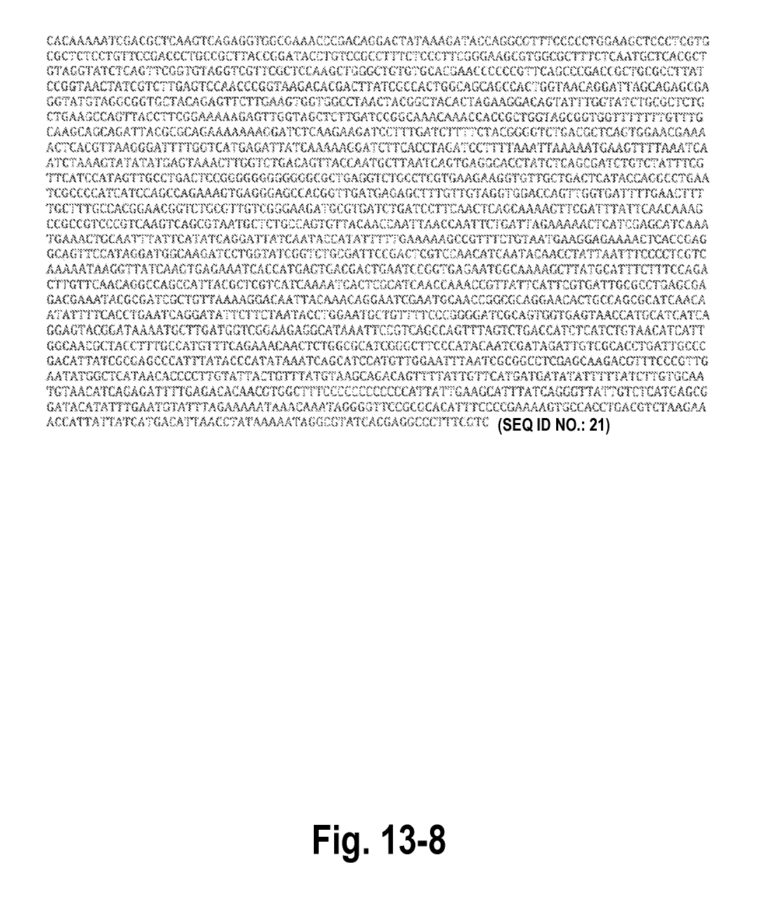

To produce a genetically modified Fn domain, a heterologous amino acid sequence, an accessory sequence, a linker, or any other component of the engineered proteins described herein, nucleic acid sequences encoding the engineered protein or a portion thereof can be ligated into an expression vector and used to transform a prokaryotic cell (e.g., bacteria) or transfect a eukaryotic (e.g., insect, yeast, or mammal) host cell. In general, nucleic acid constructs can include a regulatory sequence operably linked to a nucleic acid encoding the engineered protein or a protion thereof (see, e.g., FIGS. 6 and 13). Regulatory sequences (e.g., promoters, enhancers, polyadenylation signals, or terminators) can be included as needed or desired to affect the expression of a nucleic acid sequence. The transformed or transfected cells can then be used, for example, for large or small scale production of the engineered protein by methods well known in the art. In essence, such methods involve culturing the cells under conditions suitable for production of the engineered protein and isolating the protein from the cells or from the culture medium. Additional guidance can be obtained from the Examples presented below.

Pharmaceutical Preparations and Methods of Treatment:

The engineered proteins described herein can be administered directly to a mammal. Generally, the engineered proteins can be suspended in a pharmaceutically acceptable carrier (e.g., physiological saline or a buffered saline solution) to facilitate their delivery. Encapsulation of the polypeptides in a suitable delivery vehicle (e.g., polymeric microparticles or implantable devices) may increase the efficiency of delivery. A composition can be made by combining any of the peptides provided herein with a pharmaceutically acceptable carrier. Such carriers can include, without limitation, sterile aqueous or non-aqueous solutions, suspensions, and emulsions. Examples of non-aqueous solvents include mineral oil, propylene glycol, polyethylene glycol, vegetable oils, and injectable organic esters. Aqueous carriers include, without limitation, water, alcohol, saline, and buffered solutions. Preservatives, flavorings, and other additives such as, for example, antimicrobials, anti-oxidants (e.g., propyl gallate), chelating agents, inert gases, and the like may also be present. It will be appreciated that any material described herein that is to be administered to a mammal can contain one or more pharmaceutically acceptable carriers.

Any composition described herein can be administered to any part of the host's body for subsequent delivery to a target cell. A composition can be delivered to, without limitation, the brain, the cerebrospinal fluid, joints, nasal mucosa, blood, lungs, intestines, muscle tissues, skin, or the peritoneal cavity of a mammal. In terms of routes of delivery, a composition can be administered by intravenous, intracranial, intraperitoneal, intramuscular, subcutaneous, intramuscular, intrarectal, intravaginal, intrathecal, intratracheal, intradermal, or transdermal injection, by oral or nasal administration, or by gradual perfusion over time. In a further example, an aerosol preparation of a composition can be given to a host by inhalation.

The dosage required will depend on the route of administration, the nature of the formulation, the nature of the patient's illness, the patient's size, weight, surface area, age, and sex, other drugs being administered, and the judgment of the attending clinician. Suitable dosages are in the range of 0.01-1,000 μg/kg. Wide variations in the needed dosage are to be expected in view of the variety of cellular targets and the differing efficiencies of various routes of administration. Variations in these dosage levels can be adjusted using standard empirical routines for optimization, as is well understood in the art. Administrations can be single or multiple (e.g., 2- or 3-, 4-, 6-, 8-, 10-, 20-, 50-, 100-, 150-, or more fold). Encapsulation of the engineered proteins in a suitable delivery vehicle (e.g., polymeric microparticles or implantable devices) may increase the efficiency of delivery.

As is known in the art, dosage may vary based on the condition to be treated. One of ordinary skill in the art wishing to use an engineered protein of the present invention can obtain information and guidance regarding dosage from currently available antibody therapeutics. For example, cetuxamab, when used for the treatment of colorectal cancer in adults is delivered IV at 400 mg/m² as an initial loading dose administered as a 120-min infusion (max rate of infusion, 10 mg/min). The weekly maintenance dose is 250 mg/m² infused over 60 min (max rate of infusion, 10 mg/min) until disease progression or unacceptable toxicity. For treatment of squamous cell carcinoma of the head and neck, in adults, the recommended delivery for cetuxamab is IV in combination with radiation therapy. The recommended dose is 400 mg/m² as a loading dose given as a 120-min infusion (max infusion rate, 10 mg/min) 1 wk prior to initiation of a course of radiation therapy. The recommended weekly maintenance dose is 250 mg/m² infused over 60 min (max infusion rate, 10 mg/min) weekly for the duration of radiation therapy (6 to 7 wk). Complete administration 1 h prior to radiation therapy. As a single agent, the recommended initial dose is 400 mg/m² followed by 250 mg/m² weekly (max infusion rate, 10 mg/min) until disease progression or unacceptable toxicity. We expect the engineered proteins described herein may be beneficially administered using the same or similar regimes.

A potential advantage of the present engineered proteins is that their multispecific (e.g., heterobivalent) nature combines the efficacy of multiple compounds into a single drug, and this may reduce the total required drug dosage and facilitate administration while allowing for complementary mechanisms to synergize.

The duration of treatment with any composition provided herein can be any length of time from as short as one day to as long as the life span of the host (e.g., many years). For example, an engineered protein can be administered once a week (for, for example, 4 weeks to many months or years); once a month (for, for example, three to twelve months or for many years); or once a year for a period of 5 years, ten years, or longer. It is also noted that the frequency of treatment can be variable. For example, the present engineered proteins can be administered once (or twice, three times, etc.) daily, weekly, monthly, or yearly.

An effective amount of any composition provided herein can be administered to an individual in need of treatment. The term “effective” as used herein refers to any amount that induces a desired response while not inducing significant toxicity in the patient. Such an amount can be determined by assessing a patient's response after administration of a known amount of a particular composition. In addition, the level of toxicity, if any, can be determined by assessing a patient's clinical symptoms before and after administering a known amount of a particular composition. It is noted that the effective amount of a particular composition administered to a patient can be adjusted according to a desired outcome as well as the patient's response and level of toxicity. Significant toxicity can vary for each particular patient and depends on multiple factors including, without limitation, the patient's disease state, age, and tolerance to side effects.

Any method known to those in the art can be used to determine if a particular response is induced. Clinical methods that can assess the degree of a particular disease state can be used to determine if a response is induced. The particular methods used to evaluate a response will depend upon the nature of the patient's disorder, the patient's age, and sex, other drugs being administered, and the judgment of the attending clinician.

As noted above, the engineered proteins can also be used as delivery agents to deliver cargo (e.g., a therapeutic agent) to a particular cell type. The cargo can be internalized by virtue of internalization of the engineered protein and its target molecule. The cargo can be a cytotoxic agent, which refers to a substance that inhibits or prevents the function of cells and/or causes destruction of cells. Cytotoxic agents include radioactive isotopes (e.g., ¹³¹I, ¹²⁵I, ⁹⁰Y and ¹⁸⁶Re), chemotherapeutic agents, and toxins such as enzymatically active toxins of bacterial, fungal, plant or animal origin or synthetic toxins, or fragments thereof. The agents can also be non-cytotoxic, in which case they will not inhibit or prevent the function of cells and/or will not cause destruction of cells. A non-cytotoxic agent may include an agent that can be activated to be cytotoxic. A non-cytotoxic agent may include a bead, liposome, matrix or particle (see, e.g., U.S. Patent Publications 2003/0028071 and 2003/0032995 which are hereby incorporated by reference herein in their entireties). Such agents may be conjugated, coupled, linked or otherwise associated with an engineered protein disclosed herein.

Kits and Other Compositions:

The engineered proteins, domains thereof, nucleic acids, including vector constructs that can be used to produce them, and any of the other compositions of the invention can be packaged in various combinations as a kit, together with instructions for use.

Nucleic acid sequences encoding representative Fn3-Fn3 and Ab-Fn3 fusions are shown in FIGS. 6 and 13, respectively. These sequences and sequences that are identical to one or more defined portions therein are within the scope of the present invention. The beginnings and ends of the sequences presented in these figures are marked. For example, Fn3₁₀₁ is demarkated in FIG. 6 and Fn Clone D and 225HC. Accordingly, the invention encompasses nucleic acid constructs comprising one or more of Clone A, Clone B, Clone C, Clone D, or Clone E or biologically active fragments or other variants (e.g., substitution mutants) thereof. Other constructs can comprise the linker or leader sequences shown. For example, the invention encompasses nucleic acid constructs that include a sequence encoding a leader sequence (e.g., ATG . . . GCT of gWiz 225 HN-D), a sequence encoding a genetically modified Fn domain (e.g., Clone D (GTT . . . CAG of gWiz 225 HN-D)), a sequence encoding a linker (e.g., (Gly₄Ser)₂), and a sequence encoding a target-specific protein scaffold (e.g., 225 HC(CAG . . . GCT of gWiz 225 HN-D). Also within the scope of the invention are degenerate variants and codon optimized variants of the nucleic acids shown in FIGS. 6 and 13. Also within the scope of the invention are constructs comprising nucleic acid sequences that exhibit a certain degree of identity to the sequences shown in FIG. 6 or FIG. 13 (e.g., sequences that are at least 85% (e.g., 90%, 95%, or 98%) identical to a sequence shown in FIG. 6 or FIG. 13) and that encode proteins that retain sufficient biological activity to be useful in one or more of the methods described herein. Proteins encoded by the nucleic acid sequences shown in FIG. 6 or FIG. 13, and biologically active fragments or other variants thereof (e.g., proteins that are at least 85% (e.g., 90%, 95%, or 98%) identical to a protein encoded by a sequence shown in FIG. 6 or FIG. 13) are also within the scope of the present invention. The nucleic acid sequences described herein can be incorporated into a vector (e.g., an expression vector such as a plasmid or a cosmid or other viral vector) using methods known in the art. The nucleic acids and/or vectors that contain them can similarly be transfected into cells (e.g., cells in tissue culture), and such cells are within the scope of the present invention.

The studies described in the examples below illustrate the compositions and methods of the invention without limitation.

### EXAMPLES

In the work described below and elsewhere in this specification, we describe a platform for engineering fibronectin domains (e.g., Fn3 domains) as selective binding reagents and methods of assembling these domains, with or without heterologous amino acid sequences, to produce heterovalent proteins that specifically bind a variety of molecular targets. Several elements of protein engineering by directed evolution were improved to produce a platform enabling the identification of high affinity binders derived from the Fn3 scaffold. These technological developments are broadly applicable to the field of protein engineering.

In the studies below, we describe the production of engineered proteins that bind eight targets: the cancer targets human EGFR, human A33, mouse A33, and mouse CD276; immunological targets Fcγ receptors IIa and IIIa; and biotechnological targets mouse IgG and human serum albumin (HSA). In addition, binders to lysozyme, carcinoembryonic antigen, goat IgG, and rabbit IgG were engineered during platform development. EGFR binders were incorporated into both a novel bispecific format, where the engineered proteins feature two genetically modified Fn3 domains, and into an Fn3-Ab fusion. Selective non-competitive heterobivalent constructs are capable of receptor downregulation. Select constructs inhibit cell proliferation and migration, particularly in combination with a ligand-competitive antibody, and therefore have strong therapeutic potential.

### Example 1

### Stability and Complementarity Bias Improve the Protein Functionality Landscape

We sought to develop an improved Fn3 library design through incorporation of two key features: (a) wild-type conservation of residues that are (i) structurally important if not critical and/or (ii) conservation of residues that are less likely to contribute to the desired binding interaction and (b) tailored amino acid diversity biased to functional amino acids.

Despite their location in the BC/DE/FG loop region of Fn3, some residues may be critical to the conformational stability of the protein fold. As such, diversification of these positions may produce a library population with reduced average stability. Destabilization limits the robustness of binders in biotechnology applications such as the stringent washing steps of purification and detection. Instability can result in degradation and aggregation of in vivo diagnostics and therapeutics, which reduces potency and can elicit an immune response. Moreover, destabilization decreases the tolerance to mutation, which decreases the capacity for evolution (Bloom et al., Proc. Natl. Acad. Sci. USA, 103:5869-5874, 2006). Also, the potentially resultant flexibility may diminish the free energy change upon binding because of entropic effects. Moreover, conservation at structurally critical positions enables diversity to be focused on positions that are more likely to contribute to the binding interaction yielding a more efficient search of sequence space. In our work, we use stability, structural, and sequence analyses to identify conservation sites that may benefit library design.

Early library designs commonly used NNB or NNS/NNK randomized codons to approximate an equal distribution of all amino acids. Yet, because not all amino acids are equivalent in their ability to provide conformational and chemical complementarity for molecular recognition, a tailored distribution may be more effective. Sidhu and colleagues have investigated this hypothesis and demonstrated the utility of a tyrosine/serine library as well as the unique efficacy of tyrosine to mediate molecular recognition in antibody fragments (Fellouse et al., Proc. Natl. Acad. Sci. USA, 101:12467-12472 (2004); Fellouse et al., J. Mol. Biol., 348:1153-1162 (2005); Fellouse et al., J. Mol. Bio., 357:100-114 (2006)). Direct competition of full diversity and tyrosine/serine diversity libraries in the Fn3 domain was dominated by the full diversity library for selection of high affinity binders to goat and rabbit immunoglobulin G (Hackel and Wittrup, submitted). Thus, though tyrosine/serine may provide ample diversity for binding, an expanded repertoire enables higher complementarity. The expanded repertoire can be effectively utilized with an efficient library design and/or affinity maturation scheme. A tailored antibody library with elevated tyrosine, glycine, and serine and low levels of all other amino acids except cysteine was superior to a tyrosine/serine library (Fellouse et al., J. Mol. Biol., 373:924-940, 2007). A similarly biased library was used with the Fn3 scaffold to yield a 6 nM binder to maltose binding protein (Gilbreth et al., J. Mol. Biol, 381:407-418, 2008) and a novel ‘affinity clamp’ for peptide recognition (Huang et al., Proc. Natl. Acad. Sci. USA, 2008). These biased distributions were created by oligonucleotide synthesis using custom trimer phosphoramidite mixtures. Our current work investigates the ability to create a desired distribution via inexpensive skewed nucleotide mixtures. In particular, the amino acid distribution in human and mouse CDR-H3 loops is effectively mimicked. We demonstrate that a new library incorporating selective conservation and tailored diversity is superior to both an unbiased library with approximately equal amino acid diversity and a tyrosine/serine binary code library. This library enabled the generation of binders to a multitude of targets with expected utility in research, biotechnology, and therapy.

Fn3 Stability:

We used yeast surface display for efficient stability analysis of Fn3 clones. It has been demonstrated that the number of displayed single-chain T-cell receptors per yeast cell correlates to receptor stability (Shusta et al., J. Mol. Biol, 292:949-956 (1999)). To validate this correlation for Fn3, we created yeast surface display vectors of binders to vascular endothelial growth factor receptor 2 spanning a range of stabilities: free energies of unfolding from 3.8 to 7.5 kcal/mol and midpoints of thermal denaturation of 42 to 84° (Parker et al., Protein Engineering Design and Selection, 18:435-444 (2005)). Clonal cultures of yeast were grown at 30° C., Fn3 expression was induced at 37° C., and the amount of displayed Fn3 was quantified by flow cytometry. The clones exhibit a positive relationship between display and stability spanning a substantial display range between the least and most stable clones, thereby validating this technique for stability comparison.

This validated approach was used to explore domain stabilization via single-site wild-type conservation in the context of a diverse library. To quantify this impact, a series of libraries were constructed: one library with fully diversified BC, DE, and FG loops and multiple libraries of the same design except for wild-type conservation at a single position of interest. The libraries were transformed into a yeast surface display system and the amount of Fn3 displayed upon induction at 37° C. was quantified by flow cytometry. Eleven of fourteen positions studied, as well as a multisite library, exhibit improved display with wild-type conservation. A26, V27, and T28 have increased display but not of statistical significance. Accordingly, Fn domains useful in the presently engineered proteins include those in which one or more of the residues at positions 23, 24, 25, 29, 52, 56, 77, 78, 79, 84, and 85 of SEQ ID NO:1 are conserved. Analogous positions in fibronectins from other species can be also be conserved.

Solvent Accessible Surface Area:

The solvent accessible surface area of each potentially diversified position was calculated using GetArea (Fraczkiewicz and Braun, J. Computational Chemistry, (1998)) for wild-type Fn3 (solution structure 1TTG (Main et al., Cell, 71:671-678 (1992)) and crystal structures 1FNA (Dickinson et al., J. Mol. Biol, 236:1079-1092 (1994))) and an engineered binder (20BG (Koide et al., Proc. Natl. Acad. Sci. USA, 104:6632-6637 (2007))). Despite their presence in previously diversified loop regions, the side chains of D23, A24, P25, V29, G52, and S85 are relatively inaccessible; peripheral residues W22, Y32, A57, T76, and P87 are also buried. Conversely, the amino acids in the middle of each loop are relatively exposed, supporting the ability of these sites to be diversified while maintaining the correct fold.

Sequence Analysis:

The mutational flexibility of each position was further explored through phylogenetic sequence analysis. The type III domains of fibronectin in chimpanzee, cow, dog, horse, human, mouse, opossum, platypus, rat, and rhesus monkey (any of which can serve as starting materials for an engineered protein as described herein) were aligned, and the relative frequency of each amino acid was determined (FIG. 1(A)). The peripheral residues W22, Y32, P51, A57, and P87 are well conserved; however, T76 is variable. Other sites exhibiting conservation three-fold above random are A24 (22%), P25 (62%), V29 (25% as well as 43% isoleucine), G52 (25%), S53 (23%), S55 (27%), G77 (21%), G79 (19%), and S85 (66%); also note that T56 is 12% conserved with 51% of the homolog serine. Thus, the BC loop exhibits conservation of its peripheral hydrophobic residues except Y31. The DE loop, except for the central lysine, is well-conserved. The FG loop has a trend towards glycine from G77 to G79 and two highly conserved sites near the C-terminus,

Published sequences of engineered binders were analyzed similarly. However, in this analysis, amino acid frequencies were compared to expected frequencies based on variable library designs (FIG. 1(B)). Wild-type is present at least twice as often in binders as in the naïve library at three positions: P25 (15% in binders versus 5% in libraries), G52 (26% v. 13%), and G79 (17% v. 5%). In addition, three positions yield substantial enrichment of homologs: alanine at V29 (20% v. 6%), threonine at S55 (25% v. 6%), and serine at T56 (28% v. 11%).

Library Design:

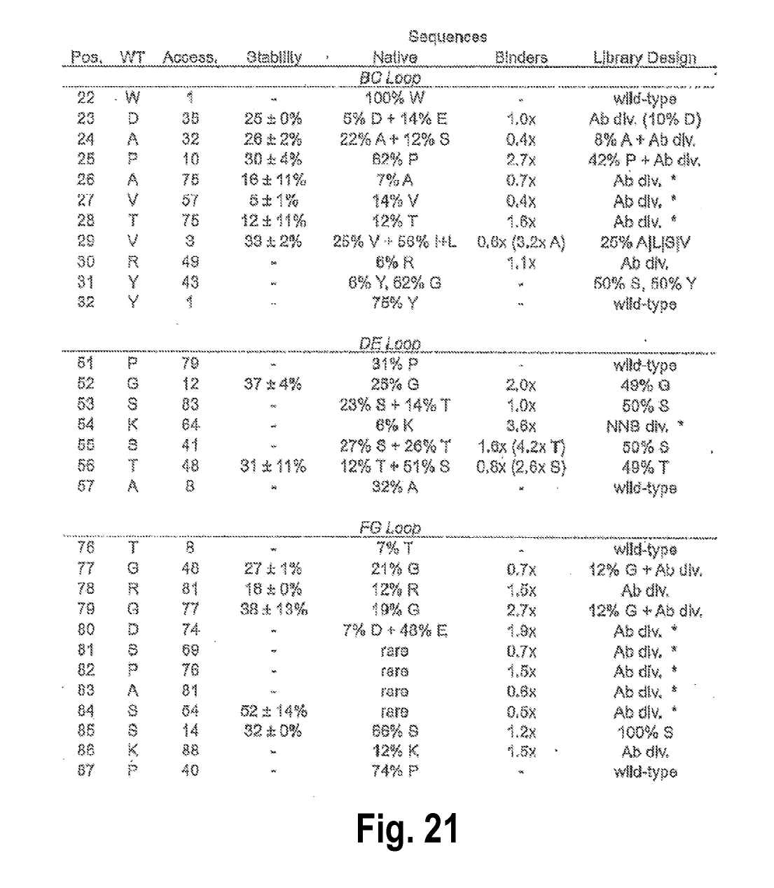

The stability, accessibility, and sequence analyses (summarized in the Table of FIG. 21) were used to determine the degree of diversification desired at each position. For example, proline at position 25 significantly stabilizes the library, is essentially inaccessible to solvent, and is highly conserved in the type III fibronectin domains of mammals. Thus, the new library will be heavily biased towards proline at this position. Conversely, the adjacent alanine at position 26 does not significantly stabilize the library, is highly accessible, and exhibits essentially no conservation. As a result, this position will be fully diversified in the new library design.

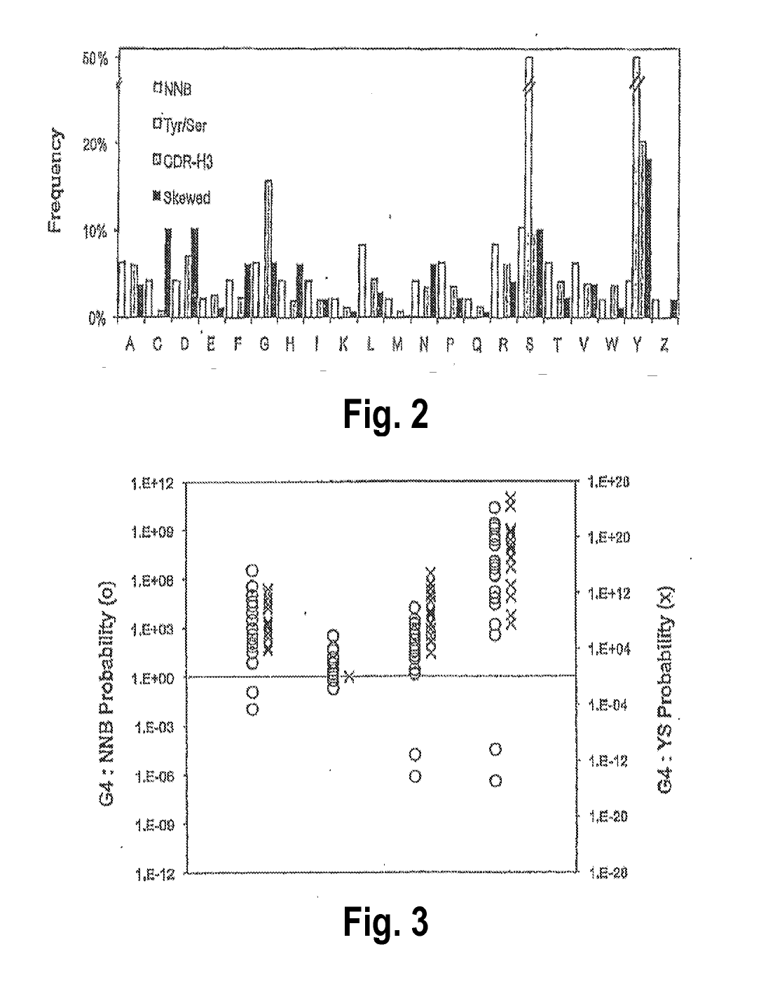

Along with conservation bias to maintain structural integrity and focus diversity on positions better suited for molecular recognition, it was desired to bias the diversity to functional amino acids. Tyrosine has demonstrated unique utility in molecular recognition (Fellouse et al., Proc. Natl. Acad. Sci. USA, 101:12467-12472, 2004; Fellouse et al., J. Mol. Biol, 348:1153-1162, 2005; Fellouse et al., J. Mol. Bio., 357:100-114, 2006). Glycine provides conformational flexibility. Serine and alanine are valuable as small, neutral side chains. Acidic residues, arginine, and lysine provide charge although the utility is unclear (Birtalan et al., J. Mol. Biol, 377:1518-1528, 2008). Other side chains may provide ideal complementarity in less frequent situations. Thus, we propose the ideal diversity contains high tyrosine, glycine, and serine and/or alanine as well as small levels of all other amino acids. For the particular amino acid distribution we sought guidance from natural molecular recognition. The amino acid distribution in CDR-H3 matches the desired diversity and was used as the library design model (FIG. 2). Each position was designed to incorporate the desired level of wild-type conservation and to match the antibody CDR-H3 repertoire in the non-conserved portion of the distribution. The DE loop is a slight exception because a very similar design was previously validated as effective (Hackel and Wittrup, submitted. In this loop, G52, S53, S55, and T56 are highly conserved with wild-type at 50% frequency and unbiased distribution of all other amino acids. The lack of antibody-inspired bias in this loop is of limited detriment because of the high conservation of the wild-type amino acids. Multiple loop lengths, selected based on phylogenetic occurrence (Hackel et al., J. Mol. Biol., 381:1238-1252, 2008), are included in each loop. The resultant library design is summarized in the Table of FIG. 21.

Library Construction:

Though trimer phosphoramidite library construction enables precise creation of unique amino acid distributions, this approach is expensive with the inclusion of multiple specialty codon mixtures. As an inexpensive alternative, standard oligonucleotide synthesis was employed using custom mixtures of skewed nucleotides at each position. The optimal set of three nucleotide mixtures was determined for each codon as follows. All possible sets of nucleotide mixtures with each component at 5% increments were filtered to select only those that closely match the desired levels of wild-type and tyrosine and reasonably match glycine, serine, aspartic acid, alanine, and arginine; these amino acids are the most frequent in antibody CDR-H3 and are functionally diverse. Sample protein libraries were then produced in silico from the amino acid probability distributions resulting from the sets of nucleotide mixtures. The library calculated to be most likely to be produced from the intended distribution (i.e., the antibody repertoire with the appropriate wild-type bias) was selected as optimal. This process was repeated for each position in the library.

In general, these skewed nucleotide mixtures provide good matches to the desired amino acid distributions (FIG. 2). The two exceptions are decreased levels of glycine and elevated cysteine. Since the latter two positions in a cysteine codon (TGT or TGC) are shared by glycine (GGN), it is not possible to create high levels of glycine without also yielding high cysteine unless TNN codons are depleted, which depletes tyrosine. Thus, a compromise is reached with 6% glycine and 10% cysteine. Though this incorporates a relatively high level of cysteine, the library design still yields many cysteine-free clones; moreover, interloop disulfide bonds are a potentially advantageous element (Lipovsek et al., J. Mol. Biol., 368:1024-1041, 2007).

Fn3 genes were constructed by overlap extension PCR of partially degenerate oligonucleotides. Transformation into yeast by electroporation with homologous recombination yielded 2.5×10⁸ transformants. Sequencing and flow cytometry analysis indicate 60% of clones encode for full-length Fn3 resulting in 1.5×10⁸ Fn3 clones. Sequence analysis reveals that the skewed nucleotides accurately match their intended distribution (FIG. 2). The library is termed G4, as it is the fourth generation Fn3 library created in our laboratory after the two-loop, single-length BF14 library (Lipovsek et al., J. Mol. Biol., 368:1024-1041, 2007), the three-loop, length-diversified NNB library (Hackel et al., J. Mol. Biol., 381:1238-1252, 2008), and the three-loop, DE-conserved tyrosine/serine library YS (Hackel and Wittrup, submitted).

Library Comparison:

The new G4 library design was compared to a non-conserved, full diversity library (NNB (Hackel et al., J. Mol. Biol., 381:1238-1252, 2008)) and to a library with wild-type conservation in the DE loop only and tyrosine/serine diversity (YS (Hackel and Wittrup, submitted)) (see the Table below).

| Library | Loop Diversity | Biased Positions | Full-length FN3s |
|---|---|---|---|
| NNB | full diversity (NNB | none | 0.7 × 10⁸ |
| | codons) | | |
| YS | 50% Y, 50% S | 52, 53, 55, 56 | 1.5 × 10⁸ |
| G4 | antibody-based (18% | 23, 24, 25, 29, 31, | 1.5 × 10⁸ |
| | Y, 10% S, . . .) | 52, 53, 55, 56, 77, | |
| | | 79, 85 | |

“Loop Diversity” indicates the library of codons included at positions without wild-type bias. “Biased Positions” indicates positions within the diversified loops (23-31, 52-56, 77-86) that are biased towards wild-type. “Full-length Fn3s” indicates the library size (i.e., the number of yeast transformants that encode for full-length Fn3 domains).

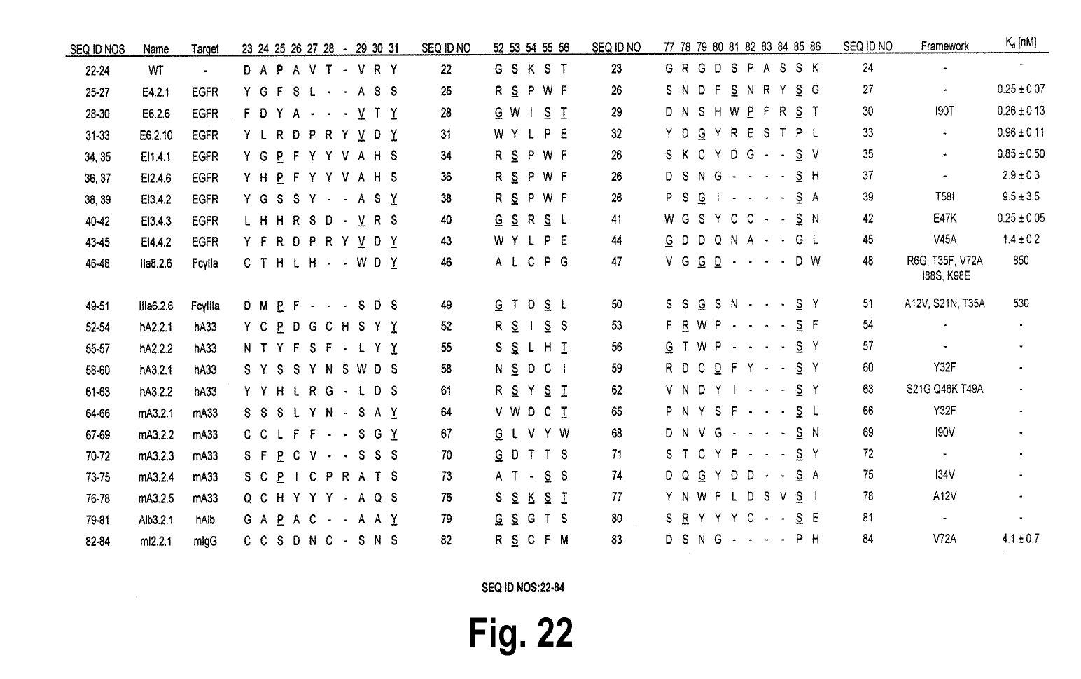

The libraries were pooled for comparison and tested for their ability to generate binders to seven targets: human A33, mouse A33, epidermal growth factor receptor (EGFR), Fcγ receptors IIA and IIIA (FcγRIIA and FcγRIIIA), mouse immunoglobulin G (mIgG), and human serum albumin (HSA). The naïve library was sorted by magnetic bead selections (Ackerman et al., Biotechnol Prog., 25:774-783 (2009)), and lead clones were diversified by error-prone PCR on the full Fn3 gene and shuffling of mutagenized Fn3 loops. Multiple rounds of selection and diversification were performed to yield binders to each target. Sequence analysis of each binding population revealed that 19 of 21 binders originated from the G4 library while two clones were likely of NNB origin and no YS clones were identified (see the Table of FIG. 22 and Figure ______). Given the comparable number of clones in the naïve libraries, this result indicates that G4 is a superior library design to both NNB and YS for the selection of protein binders.

Sequence analysis reveals that wild-type bias is approximately maintained or perhaps slightly reduced in the BC and FG loops of binders while the strong bias at G52, S55, and T56 is slightly reduced but still highly frequent. It is noteworthy that in addition to 20% occurrence at G79, glycine is present at 15% at position 80. At position 29, equal amounts of alanine, leucine, serine, and wild-type valine were included in the naïve library; in binders, the smallest available side-chain, alanine, is present at 35% while the largest side-chain, leucine, occurs with only 10% frequency. Cumulative analysis of amino acid frequency at positions without wild-type bias indicates maintenance of the preferentially high levels of tyrosine, serine, glycine, aspartic acid, and arginine. Conversely, cysteine and histidine, which were included at higher frequency than intended because of their codon similarity to tyrosine, are present at reduced levels in binders. Eight of nineteen (42%) G4-based binders are cysteine-free as compared to 19% in the naïve library. Interestingly, only three clones (16%) have a single cysteine as compared to a naïve 33% whereas seven clones (37%) contain two cysteines (26% in naive library). A single clone has four cysteines. Thus, a strong selective pressure exists against unpaired cysteines. Of particular interest, six of the seven two-cysteine clones contain cysteine residues in identical or adjacent loops at proximal positions suggesting feasible disulfide bonding, which can stabilize the domain (Lipovsek et al., J. Mol. Biol., 368:1024-1041, 2007). Thus, both wild-type bias and tailored diversity were effective in producing an effective library. Additional engineering campaigns and sequence analysis will improve the statistical significance of these trends and guide further library improvement.

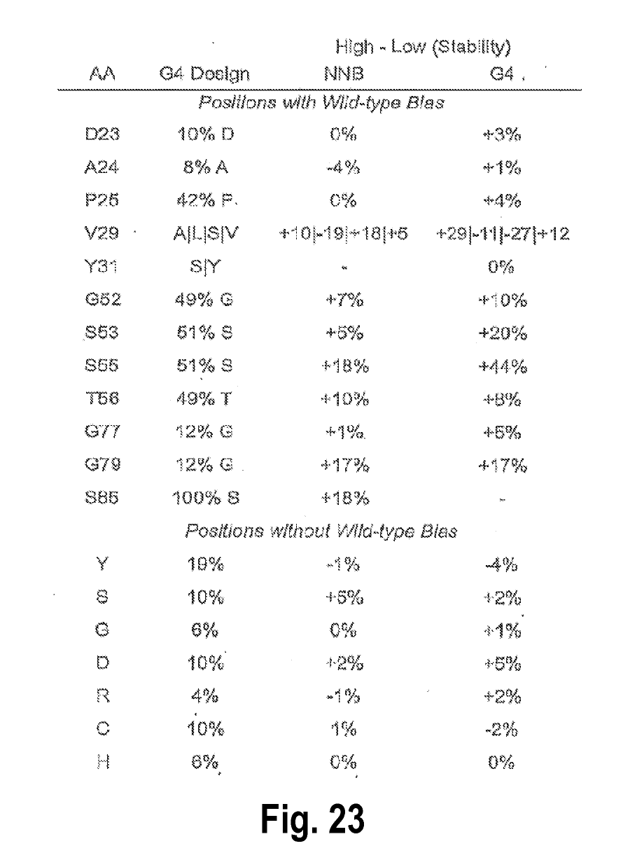

The impact of wild-type bias and tailored diversity on domain stability was analyzed. The NNB and G4 libraries were each induced for yeast surface display at elevated temperature (37° C.). The G4 library exhibits 43±9% higher average display than the NNB library indicating higher average stability; clones from G4 are substantially more stable than those from NNB. The libraries were then sorted by FACS to identify clones of low stability and high stability. About 50 clones were sequenced from each resultant population and the amino acid frequencies in low and high stability clones were compared (see the Table of FIG. 23). The biased positions in the BC loop were not critical to stability in this analysis except position 29. As observed in binder sequence analysis, the small side chain alanine is preferred whereas the larger side chain leucine is destabilizing. Wild-type amino acids at the four biased positions in the DE loop are stabilizing, especially S53 and S55. While G77 is perhaps mildly stabilizing, G79 is present at substantially higher frequency in stable clones. The complete conservation of S85 in the G4 library is justified by the preferential occurrence of S85 in stable clones from the NNB library. At positions without wild-type bias, none of the preferred amino acids are substantially destabilizing thereby validating their inclusion at elevated levels.

Discussion:

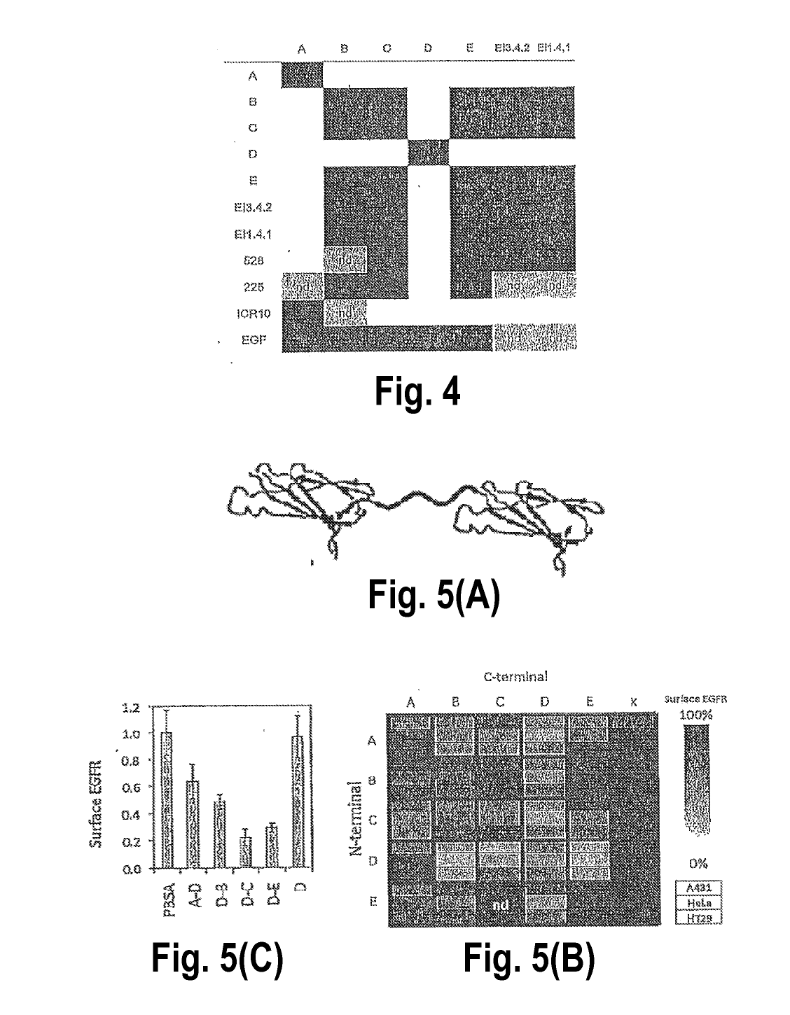

The current work demonstrates that tailored diversity is superior to nearly fully random (e.g., NNB) or overly constrained (e.g., YS) diversity. This is evidenced by the dominant selection of clones from the G4 library as well as the maintenance of the favored amino acids in binder sequences (FIG. 4.6(B)). Tailored diversity improves the search of sequence space by increasing the frequency of functional binders. This results both through improving the likelihood of beneficial contacts, largely by elevation of tyrosine, and reducing detrimental constraints. The latter element is achieved through reduction of hydrophobic isoleucine, leucine, methionine, proline, threonine, and valine as well as the large, positively charged arginine and lysine, in deference to small, neutral serine. Yet a binary code of tyrosine and serine constrains sequence space such that it often lacks high affinity binders. Thus, through modest incorporation of other amino acids in the library and a broad, yet efficient mutagenesis approach, tailored diversity yields a vastly improved hybrid of the two extremes of NNB and YS.

The inclusion of wild-type bias is also an important element of the G4 library design. This bias increases the frequency of functional clones both by enabling diversity to be used at positions with more impact on binding and by reducing the number of non-functional clones that result from detrimental mutation of a structurally critical residue. Moreover, the improved stability of G4 clones improves evolvability (Bloom et al., Proc. Natl. Acad. Sci. USA, 103:5869-5874, 2006) allowing otherwise unstable sequence motifs to be explored.

The methodology and techniques in the current work are directly applicable to any protein engineering effort. While the designed skewed nucleotide mixtures for particular sites are unique to Fn3, the antibody mimic mixture should be generally applicable to solvent-exposed loops in molecular recognition scaffolds. Moreover, the mixture design algorithm may be reapplied to any design distribution. The identification of positions most likely to benefit from wild-type bias can be readily applied to other scaffolds through high throughput stability analysis in the context of protein libraries, demonstrated here using yeast surface display. When available, sequence and structural data provide additional avenues of analysis. The relative efficacy of each of these approaches will be elucidated as continued analyses expand the sequence data set and evolved library designs are tested.

Though the thrust of this work entails study of sequence/structure/function relationships and library design, the panel of binders generated provides useful reagents for a variety of applications from tumor targeting (EGFR, human A33, and mouse A33) to biotechnology (HSA and mouse IgG) to immunology (FcγRIIa and FcγRIIIa). In addition, binders to tumor vasculature target CD276 were engineered solely from the G4 library.

In the paragraphs that follow, we describe the materials and methods that were employed in more detail.

Stability-Display Relationship:

Yeast surface display plasmids were created for six Fn3 domains of previously published stabilities: wild-type, 159, 159(wt DE), 159(Q8L), 159(A56E), and 159(Q8L,A56E) (Parker et al., Protein Engineering Design and Selection, 18:435-444 (2005)). Genes were constructed by overlap extension PCR of eight oligonucleotides and transformed into EBY100 yeast as described (Hackel et al., J. Mol. Biol, 381:1238-1252 (2008)). Gene construction was verified by DNA sequencing. Clonal populations were grown at 30° C. in SD-CAA medium (0.07M sodium citrate pH 5.3, 6.7 g/L yeast nitrogen base, 5 g/L casamino acids, and 20 g/L glucose) and induced at 37° in SG-CAA (0.1M sodium phosphate, pH 6.0, 6.7 g/L yeast nitrogen base, 5 g/L casamino acids, 19 g/L galactose, and 1 g/L glucose). Yeast were labeled with mouse anti-c-myc antibody (clone 9E10) followed by phycoerythrin-conjugated goat anti-mouse antibody. Yeast were washed and phycoerythrin fluorescence was analyzed with an Epics XL flow cytometer (Beckman Coulter, Fullerton, Calif.).

Library Stability Analysis:

A library was constructed in which positions 23-30 (DAPAVTVR (SEQ ID NO:______)), 52-55 (GSKST (SEQ ID NO:______)), and 77-86 (GRGDSPASSK (SEQ ID NO:______) were diversified using NNB codons. The library was constructed by overlap extension PCR of eight oligonucleotides and transformed into EBY100 yeast. Fourteen similar libraries were constructed with identical design except a single codon of interest was maintained as wild-type within the otherwise diversified regions. Separate libraries were constructed for D23, A24, P25, A26, V27, T28, V29, G52, T56, G77, R78, G79, S84, and S85; in addition, a library was constructed that maintained D23, A24, P25, and V29. These libraries, as well as wild-type Fn3, were grown at 30° C. and induced at 37°; Fn3 expression was analyzed by flow cytometry as indicated above. The fractional improvement in display was calculated as the mean phycoerythrin fluorescence of the singly-conserved library minus that of the fully-diversified library and normalized to the fully-diversified fluorescence.

Solvent-Accessible Surface Area:

The relative solvent accessible surface area of positions 22-32, 51-57, and 76-87 were calculated for wild-type Fn3 (solution structure 1TTG (Main et al., Cell, 71:671-678, 1992) and crystal structures 1FNA (Dickinson et al., J. Mol. Biol., 236:1079-1092, 1994) and an engineered binder (2OBG (Koide et al., Proc. Natl. Acad. Sci. USA, 104:6632-6637, 2007). The area accessible to a 1.4 Å sphere was determined for each side chain in each structure and compared to the accessible area in a G-X-G random coiled peptide using GetArea (Fraczkiewicz and Braun, J. Computational Chemistry, 1998).

Phylogenetic Sequence Alignment:

The following fibronectin sequences were used: chimpanzee (XP_{—}516072), cow (P07589), dog, (XP_{—}536059), horse (XP_{—}001489154), human (NP_{—}997647), mouse (NP_{—}034363), opossum (XP_{—}001368449), platypus (XP_{—}001509150), rat (NP_{—}062016), and rhesus monkey (XP_{—}001083548). The sequences were aligned using ClustalW (Larkin et al., Bioinformatics, Version 2.0 (2007)). The relative frequency of each amino acid was calculated at each position.

A similar analysis was conducted using engineered binder sequences. Engineered Fn3 domain sequences were aligned (sequences as in: Hackel and Wittrup, submitted; Gilbreth et al., J. Mol. Biol., 381:407-418, 2008; Huang et al., Proc. Natl. Acad. Sci. USA, 2008; Parker et al., Protein Engineering Design and Selection, 18:435-444, 2005; Koide et al., Proc. Natl. Acad. Sci. USA, 104:6632-6637, 2007; Hackel et al., J. Mol. Biol, 381:1238-1252, 2008; Lipovsek et al., J. Mol, Biol, 368:1024-1041, 2007; Koide et al., J. Mol. Biol, 284:1141-1151, 1998; Koide et al., Proc. Natl. Acad. Sci. USA, 99:1253-1258, 2002; Xu et al., Chemistry & Biology, 9:933-942, 2002; Karatan et al., Chemistry & Biology, 11:835-844, 2004; Olson et al., ACS Chem. Biol., 3:480-485, 2008) were aligned; identical loop sequences in related clones were only counted once to avoid bias. The amino acid frequency at each position was calculated and compared to the expected amino acid frequency as determined from a weighted average of theoretical library designs (e.g., NNS, NNB, serine/tyrosine, etc.).

Library Construction:

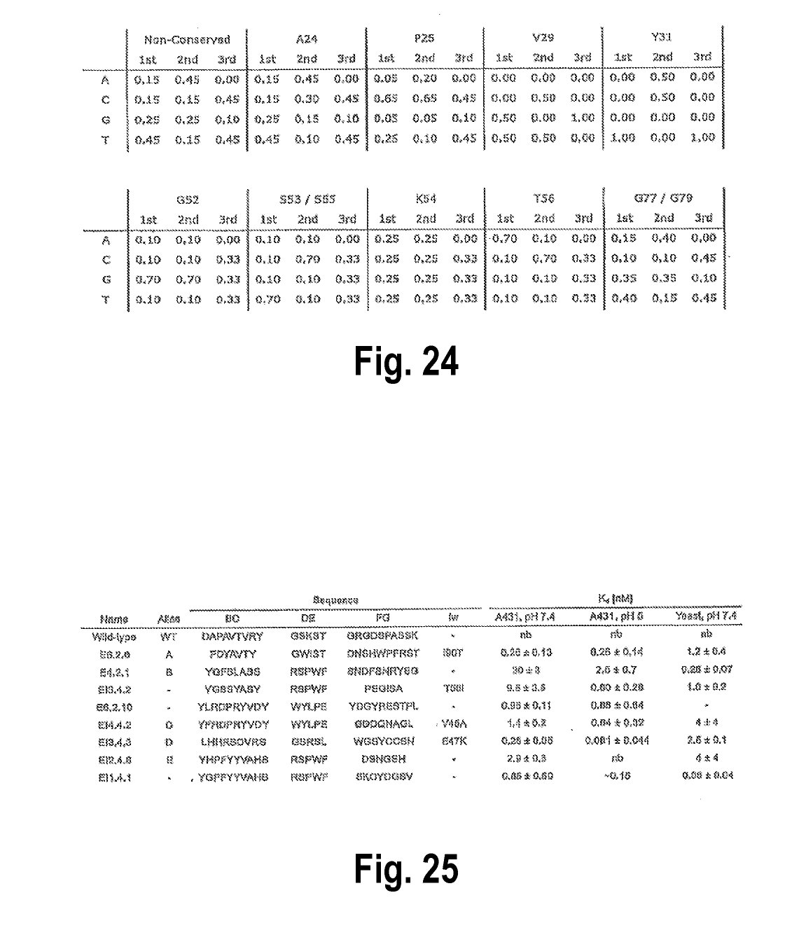

Degenerate oligonucleotides were designed to provide the desired amino acid distribution at each position. All three-site combinations of skewed nucleotide mixtures within 5% increments were considered (e.g., 20% A, 5% C, 35% G, 40% T at the first position, 15% A, 45% C, 10% G, 30% T at the second position, and 35% A, 25% C, 30% G, 10% T at the third position). The amino acid probability distribution of each set of nucleotides mixtures was calculated from the genetic code. The sets were filtered to identify those with good tyrosine matching and reasonable matching of alanine, aspartic acid, glycine, arginine, and serine. Specifically, tyrosine was required to occur at 0.5-2× the intended frequency; alanine, aspartic acid, glycine, arginine, and serine were required to occur at 0.33-3× the intended frequency. The sets that fulfilled these criteria were then used to produce numerous in silico protein libraries based on their amino acid probability distribution. For each clone, the probability of occurrence from a library that precisely matched the desired distribution was calculated. The sum of probabilities for each sample library was used as a metric of library fitness. The skewed nucleotide designs were selected based on fitness and the ability to use identical mixtures at multiple sites (e.g., 45% C, 10% G, 45% T at the wobble position of multiple codons). Nucleotide designs are included in the Table of FIG. 24.

Degenerate oligonucleotides were synthesized with skewed nucleotides at diversified positions and nucleotides encoding wild-type Fn3 at fully-conserved positions. The library design, summarized in the Table of FIG. 21, includes four, three, and four loop lengths in the BC, DE, and FG loops. Separate oligonucleotides were synthesized to yield each length. Overlap extension PCR of eight oligonucleotides was performed to construct complete Fn3 genes. Separate reactions were conducted for each loop length to avoid bias towards shorter loops. The gene libraries were transformed into yeast by homologous recombination with linearized yeast surface display vector, which includes the Aga2p protein fusion, N-terminal HA epitope, and C-terminal c-myc epitope. The fraction of clones that produce full-length Fn3 was determined by flow cytometry as the fraction displaying the N-terminal HA tag that also contained the C-terminal c-myc epitope; these results were corroborated by sequence analysis.

Binder Selections:

Human and mouse A33 extracellular domains were both produced with His₆ epitope tags in human embryonic kidney cells and purified by metal affinity chromatography. Protein was biotinylated either on free amines using the sulfo-NHS biotinylation kit or by site-specific sortase-based conjugation of GGGGG-biotin to an LPETG C-terminal epitope (Parthasarathy et al., Bioconjug. Chem. 18:469-476 (2007)). EGFR mutant 404SG (Kim et al., Proteins, 62:1026-1035 (2006)) was produced in Saccharomyces cerevisiae yeast, purified by metal affinity chromatography and anti-EGFR antibody affinity chromatography, and biotinylated on free amines using the sulfo-NHS biotinylation kit. Biotinylated FcγRIIA and FcγRIIIA were a kind gift from Jeffrey Ravetch (Rockefeller University). Biotinylated mIgG was purchased from Rockland Immunochemicals. Human serum albumin (Sigma) was biotinylated using the sulfo-NHS biotinylation kit. The NNB, YS, and G4 libraries were pooled for direct competition.

The libraries were sorted for binding to the seven protein targets and affinity matured as described (Hackel and Wittrup, submitted). Yeast were grown and induced to display Fn3. Binders to streptavidin-coated magnetic Dynabeads were removed (Ackerman et al., Biotechnol. Prog., 25:774-783 (2009)). Biotinylated protein was loaded on streptavidin-coated magnetic Dynabeads and incubated with the remaining yeast. The beads were washed with PBSA and the beads with attached cells were grown for further selection. After two magnetic bead sorts, full-length Fn3 clones were selected by fluorescence-activated cell sorting using the C-terminal c-myc epitope for identification of full-length clones. Plasmid DNA was zymoprepped from the cells and mutagenized by error-prone PCR of the entire Fn3 gene or the BC, DE, and FG loops. Mutants were transformed into yeast by electroporation with homologous recombination and requisite shuffling of the loop mutants. The lead clones and their mutants were pooled for further cycles of selection and mutagenesis. Once significant binder enrichment was observed during magnetic bead sorts, fluorescence activated cell sorting was used. Yeast displaying Fn3 were incubated with biotinylated target protein and anti-c-myc antibody (clone 9E10 or chicken anti-c-myc, Invitrogen). Cells were washed and incubated with AlexaFluor488-, phycoerythrin-, or AlexaFluor647-conjugated streptavidin and fluorophore-conjugated anti-mouse or anti-chicken antibody. Cells were washed and cells with the highest target to c-myc labeling ratio were selected on a FACS Aria or MoFlo flow cytometer. Plasmids from binding populations were zymoprepped and transformed into E. coli; transformants were grown, miniprepped, and sequenced.

Library Source Determination:

For each clone, the probabilities that it originated from the NNB, YS, or G4 library were calculated using the designed nucleotide distributions at each position as well as the probability of mutation by error-prone PCR.

Library Stability Analysis:

The NNB and G4 libraries were independently grown at 30° C. and induced at 37°. Yeast were labeled with mouse anti-HA antibody (clone 16B12, Covance) and chicken anti-c-myc antibody to label the N- and C-terminal epitopes. Cells were washed, incubated with phycoerythrin-conjugated goat anti-mouse antibody and AlexaFluor488-conjugated goat anti-chicken antibody, and sorted by flow cytometry. Only cells were comparable signals for each epitope were considered to avoid selecting epitope mutants. The lowest and highest displaying cells were collected and grown for an additional induction and selection. Plasmids were isolated and transformed into E. coli. About 50 clones from each resultant population (both low and high stability for both NNB and G4) were miniprepped and sequenced. Sequences were aligned and the amino acid frequencies at each position were determined.

### Example 2

### Epidermal Growth Factor Receptor Downregulation with Bivalent Fibronectin Constructs

An alternative mode of therapy is substantial receptor downregulation to reduce or eliminate the detrimental effects of receptor activation on tumor formation, proliferation, and migration. A previously demonstrated means of receptor downregulation is administration of non-competitive pairs of antibodies. Antibodies 528 and 806 downregulate EGFR and synergistically inhibit tumor xenografts (Perera et al., Clin. Cancer Res., 11:6390-6399, 2005). Non-competitive antibody pairs 111+565 and 143+565 downregulate EGFR whereas the competitors 111+143 do not (Friedman et al., Proc. Natl. Acad. Sci. USA, 102:1915-1920, 2005). Also, non-competitive anti-HER2 antibodies downregulate HER2 and inhibit tumor growth (Friedman et al., Proc. Natl. Acad. Sci. USA, 102:1915-1920, 2005; Ben-Kasus et al., Proc. Natl. Acad. Sci. USA, 106:3294-32999, 2009). However, these approaches require dosing two molecules, which complicates regulatory and clinical procedures. Moreover, decoupled pharmacokinetics could reduce synergy. We believe a bispecific molecule would alleviate these problems though the efficacy is uncertain given the lack of mechanistic detail in the published literature. Fn3 domains provide a good system for bispecific constructs because their single-domain architecture enables simple head-to-tail fusion, which is the natural state of Fn3 domains within complete fibronectin protein.

In the current work, we engineer a panel of small, single-domain EGFR binders to multiple identified receptor epitopes. Homo- and hetero-bivalent combinations of these binders, expressed as protein fusions (and all within the scope of the present invention), are tested for the ability to downregulate receptors in a variety of cell lines. Several molecules effectively reduce EGFR levels up to 80%. The impact of epitopes, receptor density, bivalent format, and avidity are investigated. Phosphorylation, both of receptor and downstream molecules, is examined. Inhibition of proliferation and migration through downregulation is demonstrated.

Binder Engineering:

Multiple high affinity binders to distinct epitopes of EGFR ectodomain were desired. The NNB, YS, and G4 libraries were pooled and sorted for binding to biotinylated EGFR ectodomain mutant 404SG (Kim et al., Proteins, 62:1026-1035, 2006). Two clones dominated the selection. Competition against existing anti-EGFR antibodies revealed that clone E4.2.2 is competitive with ICR10, a domain I binder, and clone E4.2.1 is competitive with 528, a domain III binder. To identify additional binders, intermediate populations were sorted for binding to EGFR ectodomain in the presence of ICR10 or 528. Five unique clones that bound ICR10-blocked EGFR were identified: EI4.4.2, EI3.4.3, EI3.4.2, EI2.4.6, and EI1.4.1. In addition, two further rounds of sorting with unblocked EGFR yielded an improved mutant of E4.2.2 named E6.2.6 and one additional clone, E6.2.10 (the Table of FIG. 25). In addition to binding soluble EGFR ectodomain produced in yeast, these eight clones all bind EGFR-expressing human epidermoid carcinoma A431 cells. The affinity of each clone was determined by titration of biotinylated Fn3 binding to A431 (on ice to prevent internalization); affinities ranged from 250 pM to 30 nM (the Table of FIG. 25). For our affinity titrations, A431 cells were incubated with 0.01, 0.1, 1 or 10 nM of biotinylated E6.2.6 or E13.4.3, then washed, labeled with streptavidin-R-phycoerythrin, and analyzed by flow cytometry.

Competition and Epitope Mapping:

Clones A-E, EI3.4.2, and EI1.4.1 bind conformationally-sensitive epitopes as evidenced by their inability to bind EGFR ectodomain after thermal denaturation of receptor on the yeast surface. To demonstrate conformational sensitivity, EGFR ectodomain mutant 404SG was displayed on the yeast surface. Cells were incubated at 80° C. for 30 minutes to denature the EGFR. Cells were labeled with biotinylated Fn3 and mouse anti-c-myc antibody followed by streptavidin-R-phycoerythrin and AlexaFluor488-conjugated anti-mouse antibody. Fluorescence was quantified by flow cytometry.

Binders were tested for the ability to compete with other clones as well as with antibodies 225, 528, and ICR10 (Figure). Clone A is competitive solely with ICR10, a known domain I binder (Cochran et al., Journal of Immunological Methods, 287:147-158 (2004)). This result was corroborated by the ability of clone A to bind the EGFR ectodomain fragment comprising amino acids 1-176 displayed on the yeast surface. Clone D is not competitive with the other Fn3s or antibodies tested. It is able to bind ectodomain fragments 294-543 and 302-503, thereby localizing the binding to domain III and the beginning of domain IV. Clones B, C, E, EI3.4.2, and EI1.4.1 compete with each other as well as antibodies 225 and 528, EGF-competitive domain III binders (except for three untested combinations; see FIG. 4). A431 cells (for 225 and EGF competition) were incubated on ice with the indicated Fn3 clone or PBSA control. AlexaFluor488-conjugates of 225 or EGF were added and cells were analyzed by flow cytometry. For all other competitions, yeast displaying EGFR ectodomain were incubated with Fn3 clone 528 or ICR10 followed by biotinylated Fn3, which was detected by streptavidin-R-phycoerythrin and flow cytometry. The black boxes indicate competition and the white boxes indicate no competition. “nd” indicates samples that were not determined. Clones A-E, as well as E6.2.10, compete with EGF for binding to A431 cells.

Higher resolution epitope mapping was performed by high throughput identification of EGFR mutations that maintain foldedness but have reduced affinity for the clone of interest (Chao et al., Journal of Molecular Biology, 342:539-550, 2004). In agreement with competition and fragment labeling, clone A binds to domain I as evidenced by its reduced binding to mutants L14H, Q16R, Y45F, and H69(QRY). The specific location in domain I provides an explanation for EGF competition as the four sites identified for clone A binding are all within 4A of EGF in the EGF/EGFR crystal structure. Clones B, C, E, and E6.2.10 all bind domain III on the portion closer to domain II, which is consistent with complementary Fn3 competition as well as EGF competition. Antibody 225 competition is reasonable for clones B, C, and E given their proximity to the cetuximab (a 225 chimera) interface. The lack of E6.2.10 competition with 225 binding is also acceptable given their disparate, though proximal, epitopes. Clone D binds near the interface of domains III and IV, which is consistent with its fragment labeling and lack of competition against 225 and clones B, C, and E. The ability of clone D to compete with EGF cannot readily be explained by direct steric inhibition given their distal binding epitopes. However, a reasonable hypothesis is that clone D binding inhibits receptor untethering that supports high affinity ligand binding. Though domains III and IV do not grossly change during untethering (Burgess et al., Molecular Cell, 12:541-552, 2003), subtle rearrangements at the domain III/domain IV interface exist. For example, amino acids 430 and 506, which are the sites identified in clone D epitope mapping, move from 19.7 Å apart in the tethered structure to 16.7 Å in the dimer.

Thus, at least three classes of binders have been engineered: clone A binds to domain I; clones B, C, and E bind domain III and are competitive with each other and antibodies 225 and 528 (as well as EI1.4.1 and EI3.4.2); clone D binds to the C-terminal portion of domain III and the N-terminal portion of domain IV and does not compete with antibodies 225 and 528 nor clones B, C, E, EI1, 4.1, or EI3.4.2.

Downregulation by Heterobivalent Constructs:

To investigate receptor downregulation via a single heterobivalent agent, Fn3 clones were linked as head-to-tail protein fusions with the native seven amino acid EIDKSPQ (SEQ ID NO ______) as well as a flexible GSGGGSGGGKGGGGT (SEQ ID NO:______) linker (FIG. 5(A)). Thirty constructs comprising all possible bivalent combinations, in both orientations, as well as monomers for five clones (identified as A-E under Alias in the Table of FIG. 25; bivalents are named N-C where N and C represent the N-terminal and C-terminal Fn3 clones) were tested. Three different EGFR-expressing human cell lines were tested: A431 epidermoid carcinoma, HeLa cervical carcinoma, and HT29 colorectal carcinoma. Cells were cultured, serum starved, and incubated with 20 nM Fn3 or Fn3-Fn3 for 6-8 hours. Cells were detached, bound agent was acid stripped, and surface EGFR was quantified by flow cytometry. Although some constructs did not modify surface EGFR levels relative to PBSA control, bivalents D-B, D-C, D-D, D-E, A-D, B-D, C-D, and E-D downregulate, yielding up to 80% reduction in surface EGFR; D-B, D-C, and D-E have the greatest effect (FIGS. 5(B) and 5(C)). Thus, we have demonstrated that particular combinations of non-competitive clones in a heterobivalent construct downregulate surface EGFR, though the D-D homobivalent does moderately reduce receptor levels. Moreover, some orders work best. For example, A-D downregulates whereas D-A does not.

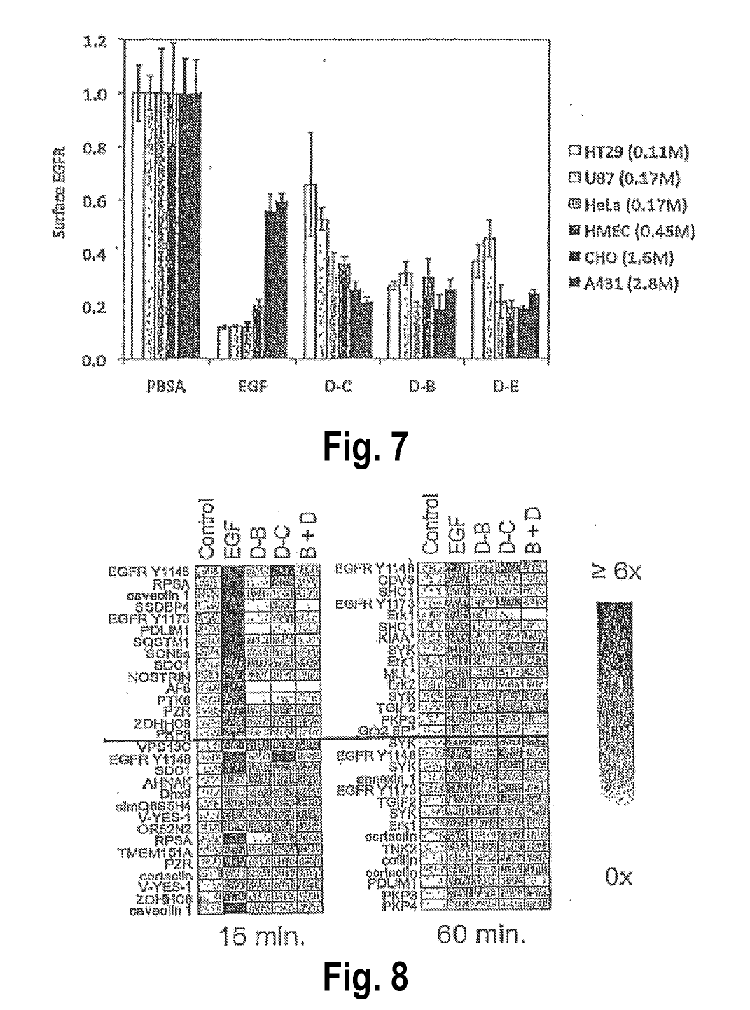

Multiple elements of downregulation were investigated. To further expand the generality of downregulation efficacy as well as to examine the impact of receptor density, three heterobivalents were tested on additional cell lines: U87 glioblastoma, hMEC (human mammary epithelial cells), and Chinese hamster ovary (CHO) cells transfected with a construct expressing an EGFR-green fluorescent protein fusion. The cells were cultured in 96-well plates, serum starved, and treated with 20 nM agent for 8 hours. Surface eGFR was quantified by flow cytometry and normalized to PBSA-treated control. Downregulation was observed in all six cell lines for D-B, D-C, and D-E (FIG. 7). Interestingly, downregulation was reduced for D-C and D-E in the low-expressing cells HT29 and U87. Conversely, EGF downregulates receptor most robustly in these low-expressing lines while exhibiting muted receptor reduction in the high-expressing CHO and A431 cells.

Downregulation kinetics were analyzed for two robust heterobivalent constructs. EGFR-expressing cells were cultured in 96-well plates, serum starved, and treated with 20 nM D-B and D-C constructs for 10 hours. Surface EGFR was measured at 2, 4, 6, 8, and 10 hours after treatment, quantitated by flow cytometry and normalized to PBSA-treated control. We found that both D-B and D-C downregulated EGFR in these A431 cells with half-times of 1.1 and 1.4 hours, respectively. Downregulation in HeLa cells is slightly faster at 0.44, 0.59, and 1.3 hours for D-B, D-C, and D-E. Thus, the genetically modified Fn3 domains of the present invention and engineered proteins containing them may effect receptor (or target) downregulation on a scale consistent with these times (e.g., with half-times of about 0.3-2.0 hours).

Heterobivalent D-C and D-E constructs were created with three different lengths of the linker between the Fn3 domains; in addition to the native EIDKPSQ sequence (SEQ ID NO ______), glycine-rich linkers of four, 15, or 27 amino acids were included. The constructs were tested for downregulation of EGFR in HT29, U87, HeLa, hMEC, CHO, and A431 cells. The cells were cultured in 96-well plates, serum starved, and treated with 20 nM of the D-C or D-E bivalent constructs for eight hours. Surface EGFR was quantified by flow cytometry and normalized to PBSA-treated control. Although results vary by cell line and by heterobivalent construct, the longer linkers were always the least effective (although still capable of receptor downregulation) and the shortest linker was often the most effective.

An alternative bispecific format was tested in which monovalent Fn3 domains were biotinylated and combinations of clones were immobilized on AlexaFluor488-conjugated streptavidin. In all bispecific and trispecific combinations of A, C, D, E, EI1.4.1, and EI3.4.2, no downregulation is observed in HT29 or U87 cells transfected to overexpress EGFR. Yet most combinations yield a substantial accumulation of internalized AlexaFluor488 signal suggestive of complex internalization without downregulation. Thus, bispecific format appears critical for efficacy. Of note, internalized AlexaFluor488 signal at 37° correlates with surface labeling at 4° (which restricts internalization) suggestive of passive internalization for all combinations.

Phosphorylation:

To investigate the mechanisms of downregulation, an EGFR expression vector was transfected into human embryonic kidney (HEK) cells, which express low levels of native EGFR. Though EGF robustly downregulates native HEK EGFR, transfected cells with approximately 50-fold more EGFR are not effectively downregulated. Conversely, D-B and D-C heterobivalents are able to downregulate transfected EGFR. The activity of the transfected EGFR is validated by a strong correlation between the fraction of cells transfected and the downregulation of native EGF; thus, the presence of overexpressing transfected cells reduces the EGF-based downregulation of non-transfected cells possibly through ligand depletion or competition. These results indicate a divergence between the mechanisms of downregulation by EGF and Fn3-Fn3 heterobivalents.

To further explore the mechanism, eight EGFR mutants with point mutations in their intracellular domains were tested for their ability to be downregulated. All eight mutants (T654A, T669A, K721R, Y845F, S1046A/S1047A, Y1068F, Y1148F, Y1173F; all of which are within the scope of the present invention) exhibit downregulation on par with wild-type EGFR in the presence of D-B and D-C.

The impact of heterobivalents on EGFR phosphorylation was analyzed at eight sites: T654, T669, Y845, S1046, Y1068, Y1086, Y1148, and Y1173. Heterobivalent D-C (20 nM), PBSA, or EGF was added to serum starved A431 cells for 5, 15, 60, or 240 minutes, and receptor phosphorylation was quantified by in-cell Western blot. The cells were fixed, permeabilized, labeled with rabbit anti-phospho-(S/T/Y) antibody followed by anti-rabbit-800CW and ToPro3 (to stain DNA), and imaged. Receptor agonism by D-C is consistently lower than that by EGF with the lone exception of T669 at early times. In fact, receptor agonism is often non-distinct from background. Thus, the genetically modified Fn domains of the invention may exhibit a general lack of agonism for a target such as the EGFR.

Likewise, standard Western blot analysis of cell lysates reveals that heterobivalents do not yield significant phosphorylation of extracellular signal-regulated kinase (ERK½) at Y202/Y204 upon 15 minute incubation whereas EGF is activating. To demonstrate ERK agonism, A431 cells were cultured in 24-well plates, serum starved, and treated with 20 nM agent for 15 minutes. Cell lysates were separated by SDS-PAGE, blotted to nitrocellulose, and labeled with rabbit anti-phosphoERK½ Y202/&204 antibody followed by peroxidase-conjugated anti-rabbit antibody and imaged.

This result is corroborated by global phosphorylation analysis of A431 cells upon addition of heterobivalent constructs for 15 or 60 minutes. The cells were treated with 20 nM agent (control, EGF, D-B, D-C, or B+D) and phosphorylated tyrosine peptides were analyzed by iTRAQ LC-MS/MS. EGF yields substantially more phosphorylation than heterobivalents or a pair of monovalents (FIG. 8).

Collectively, these data demonstrate that select Fn3-Fn3 heterobivalents substantially downregulate EGFR in a manner distinct from EGF and without significant receptor activation; there is a general lack of global agonism by the Fn-Fn constructs.

An EGFR trafficking model is shown in Appendix B of the provisional application filed Aug. 13, 2009. EGFR trafficking can be examined with a model consisting of four simple mechanisms: synthesis, endocytosis, degradation, and recycling. Constitutive synthesis produces surface receptor (S) at rate ksyn. Surface receptor is internalized to endosome (E) at rate kendoS. Endosomal receptor is degraded at rate kdegE or recycled to the surface at rate krecE.

Efficacy.

The ability of monovalent, homobivalent, and heterobivalent constructs to inhibit downstream signaling was examined. A431 cells were cultured in 24-well plates, serum starved, and treated with 20 nM agent for 6 hours. The cells were then treated with 1 nM EGF for 15 minutes. Cell lysates were separated by SDS-PAGE, blotted to nitrocellulose, and labeled with rabbit antiphosphoERK½ Y202/Y204 antibody followed by peroxidase-conjugated anti-rabbit antibody and imaged. The downregulating bivalents A-D, D-B, D-C, and D-E inhibit EGF-induced ERK phosphorylation at tyrosines 202 and/or 204 whereas non-downregulating B-B homobivalent has no effect. The monovalent EGF competitor clone D is also antagonistic. While the genetically modified Fn domains useful in the present engineered proteins are not limited to domains that work through any particular cellular mechanism, they may include those that inhibit EGF-induced ERK phosphorylation.

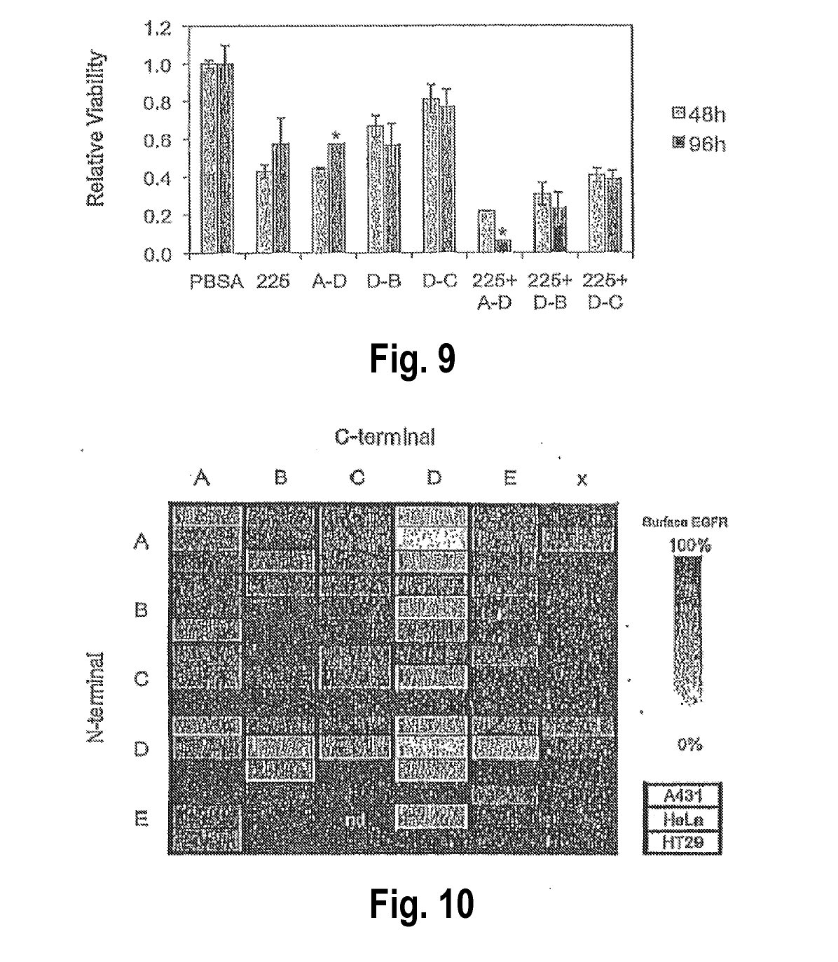

Beyond phosphorylation, the effect on cellular output was examined in terms of proliferation and migration. To test cellular output in a challenging tumor-like environment, an autocrine model system was used in which hMEC cells were transfected with a vector for a membrane-bound EGF ligand with an EGF or TGFα cytoplasmic tail (hMEC+ECT or hMEC+TCT (Joslin et al., J. Cell. Sci, 120:3688-3699, 2007)). The cells were cultured in 96-well plates and treated with 20 nM of the indicated agent(s). Additional ligand was added after 48 hours. Viability was quantified using AlamarBlue and normalized independently for each time point relative to PBSA-treated cells. Treatment with downregulating heterobivalent Fn3-Fn3 significantly reduced the number of viable cells at 48 hours and 96 hours (FIG. 9). In addition, combination treatment of 225 antibody and heterobivalent A-D further reduces cell viability. Of note, clones A and D are not competitive with 225 and thus this combination treatment elicits strong downregulation (FIG. 10). A431, HeLa, and HT29 cells were cultured, serum starved, and treated with 20 nM 225 and 20 nM of the indicated Fn3 or Fn3-Fn3 construct for 6-8 hours. Surface EGFR was quantified by flow cytometry and is presented on an intensity scale relative to PBSA-treated control. In FIG. 10, black boxes signify no downregulation, and white boxes indicate complete downregulation.

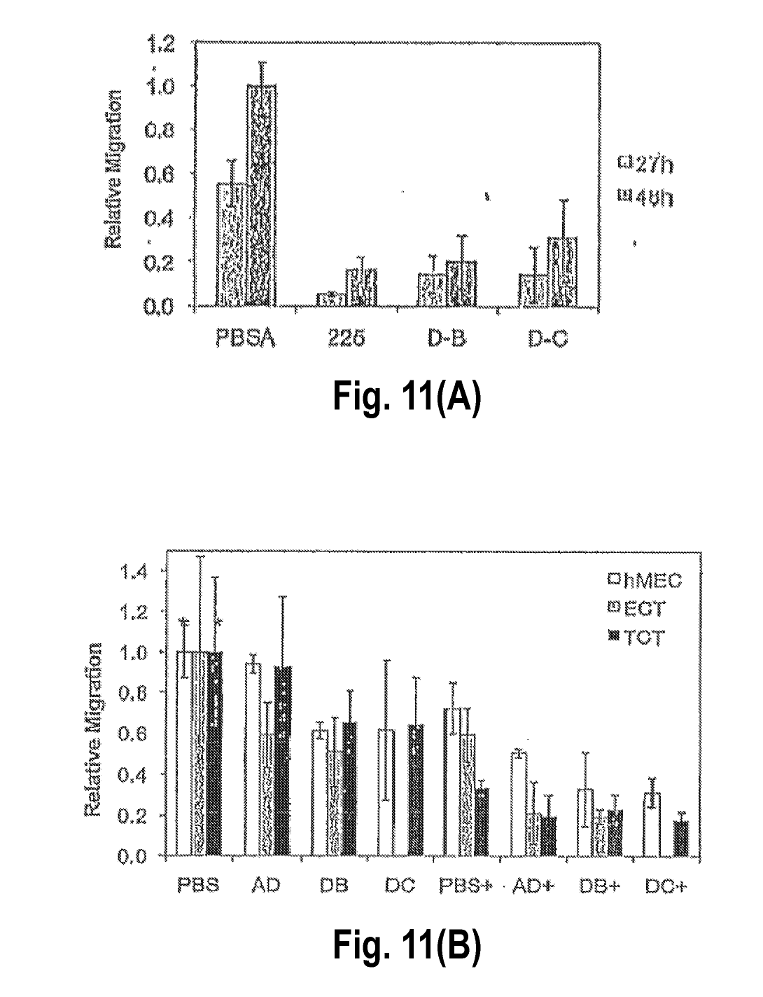

Likewise, treatment with downregulating heterobivalent constructs strongly reduces cell migration in the autocrine cells as well as parental hMEC cells, and combination treatment further augments this inhibition (FIGS. 11(A) and 11(B)). Cells were cultured in 96-well plates to a confluent monolayers. A “wound” was then scratched into each monolayer to create a void of cells. Cells were treated with 20 nM of the indicated agent(s). Migration was analyzed by microscopy. FIG. 11(A) shows the results obtained (relative migration) for hMEC cells with autocrine EGF signaling (TCT), and FIG. 11(B) shows the relative micration of hMEC, ECT, and TCT cells.

Delivery:

The engineered EGFR binders, both in monovalent and bivalent formats, are effective intracellular delivery agents. Fn3 and Fn3-Fn3 constructs were conjugated to DyLight633 fluorophore via primary amines and incubated with HT29 cells. DyLight633 readily accumulated intracellularly for EGFR binding clones but not for wild-type Fn3. Biotinylated Fn3 domains loaded onto streptavidin conjugated to AlexaFluor488 and 1.4 nm NanoGold spheres were effectively delivered to EGFR-expressing cells but not EGFR negative cells.

Discussion:

The panel of binders should provide useful reagents for a variety of applications. The small size should provide rapid clearance for in vivo imaging applications and close proximity of binding site and fluorophore for Förster resonance energy transfer studies. The engineered domains are cysteine-free with primary amines located distal to the presumed binding site with two exceptions: EI1.4.1 contains a cysteine and lysine in the FG loop and clone D contains adjacent cysteines in the FG loop. Thus, the domains are amenable to thiol and amine chemical conjugation to fluorophores, nanoparticles, drug payloads and chemically modified surfaces for drug delivery, diagnostic, and biotechnology applications. The single-domain architecture readily enables protein fusion such as the bivalents discussed herein and immunotoxins (Chris Pirie, unpublished data). The picomolar to low nanomolar binding of these domains is beneficial for most applications. The breadth of epitopes targeted is useful for biophysical studies and dual binding such as for receptor clustering or sandwich immunoassays.

The analysis of the combinations of monovalent and homo- and hetero-bivalent constructs provides a broad data set to assess the stringent criterion for downregulation. As expected, monovalent binding does not reduce EGFR levels. Homobivalents, aside from weak downregulation by D-D, also are ineffective. In fact, strong reduction in EGFR levels is only observed for select heterobivalents of non-competitive clones. Constructs D-B, D-C, and D-E yield the strongest downregulation while A-D, B-D, C-D, and E-D exhibit modest efficacy. Non-competitive heterobivalents including clone D are generally effective except for D-A. Non-competitive heterobivalents including clone A are less consistent. C-A and A-B are weakly effective against all three cell types, A-C and A-E are weakly effective against only two cell types, and B-A and E-A are ineffective. Thus, a combination of non-competitive clones is necessary but not sufficient for strong downregulation. This criterion is consistent with the purported basis for downregulation: receptor clustering. Non-competitive heterobivalent constructs can form receptor clusters because of the ability to bind two heterobivalents to a single receptor thereby propagating receptor linkages whereas homobivalents or competitive heterobivalents can only form two-receptor complexes. Meanwhile, the reduced efficacy of some non-competitive heterobivalents may arise from the inability to simultaneously bind two receptors given the distance and steric constraints of the epitopes targeted and the length and composition of the bivalent linker.

This potential mechanism is also in agreement with the reduced downregulation observed for cells expressing low levels of EGFR as reduced receptor surface density decreases the likelihood of receptor crosslinking. The origin of improved efficacy with shorter linkers is unclear. Perhaps increased conformational flexibility of the Fn3-Fn3 construct reduces the effective local concentration of the unbound Fn3 after single-receptor binding thereby decreasing crosslinking. Alternatively, shorter linkers could increase interaction of clustered receptors though significant agonism is not observed. The heterobivalents exhibit a response that is grossly different than that elicited by EGF, This is perhaps most clearly demonstrated by the ability of heterobivalents to downregulate EGFR overexpressed in HEK cells, whereas EGF does not downregulate. EGF perhaps fails because of a saturation of the cellular machinery, but regardless the mechanism of downregulation is clearly different for EGF and Fn3-Fn3. Also, multiple receptor mutants, including kinase inactive K721R, are downregulated to the same extent as wild-type receptor. Mutation of neither T669 nor S1046, whose phosphorylation is implicated in receptor internalization (Countaway et al., J. Biol. Chem., 267:1129-1140 (1992); Winograd-Katz and Levitzki, Oncogene, 25:7381-90 (2006)), nor T654, whose phosphorylation either inhibits ubiquitination or accelerates recycling (Bao et al., J. Biol. Chem., 275:26178-26186 (2000)), impacts downregulation. In addition, mutation of Y845, Y1068, Y1148, or Y1173, which are important in the ERK signaling pathway (Amos et al., J. Biol. Chem., 280:7729-7738 (2005); Biscardi et al., J. Biol. Chem., 274:8335-8343 (1999); Downward et al., J. Biol. Chem., 260:14538-46 (1985); Morandell et al., Proteomics, 8:4383-401 (2008); Wu et al., J. Biol. Chem., 277:24252-7 (2002); Yamauchi et al., Nature, 390:91-6 (1997)), has no effect. These results are corroborated by phosphorylation analyses. Of eight key sites studied on EGFR, heterobivalent D-C yielded significantly lower phosphorylation than that by EGF except at T669. Conversely, no phosphorylation is observed at T654, S1046, and Y1068. Y845, Y1086, Y1148, and Y1173 exhibit no agonism at multiple time points and weak phosphorylation at one hour. Moreover, Western blot analysis demonstrates ERK phosphorylation upon treatment with EGF but not upon treatment with any of the heterobivalents tested. Global phosphoproteomic analysis also exhibits substantially more phosphorylation from EGF than D-B, D-C, or a combination of B and D monomers. Thus, unlike EGF, Fn3-Fn3 constructs achieve receptor downregulation without significant receptor agonism.

A simple mathematical model of receptor trafficking indicates that downregulation can be expected to arise from enhanced degradation/recycling ratio, enhanced receptor internalization, or both. The lack of agonism counters the hypothesis of enhanced receptor internalization although endocytosis could be accelerated by weak phosphorylation. Alternatively, the throughput of constitutive internalization could be enhanced via receptor clustering. Yet experimental data suggest that receptor internalization is not sped as monovalent clone B and downregulating D-B exhibit equivalent intracellular accumulation. Moreover, the kinetics of downregulation (τ_{1/2}=0.4-1.4 h) are comparable to constitutive receptor internalization kinetics. Preliminary measurements of receptor internalization indicate endocytic half-times of 0.3-0.8 h (data not shown). Thus, although receptor internalization may be sped slightly, it does not appear to be the dominant source of downregulation. Enhanced degradation could conceivably result from the presence of receptor clusters that either inhibit recycling or drive degradation. In fact, AlexaFluor488-conjugated 225 antibody exhibits reduced recycling in the presence of downregulating heterobivalent A-D as compared to co-treatment with monomer A or non-downregulating C-B.

Downregulation decreases the amount of receptor available for ligand binding, receptor homo- and hetero-dimerization, and constitutive activation, thereby decreasing the opportunity for receptor signaling. Downregulation is sufficient to inhibit ERK phosphorylation, a downstream signaling molecule on a pathway that leads to proliferation and migration. Downregulating heterobivalents are shown to inhibit proliferation and migration of a cell line with autocrine signaling, and this inhibitory activity can be augmented by combination treatment with ligand-competitive antibody 225. Further study can elucidate the relative impacts of receptor downregulation and ligand competition as well as the in vivo efficacy of the heterobivalent agents.

In the paragraphs that follow, we describe the materials and methods that were employed in more detail.

Binder Engineering:

EGFR binders were engineered from the NNB, YS, and G4 pooled library comparison as outlined above. EGFR mutant 404SG^{Ref}. (Kim et al., Proteins, 62:1026-1035 (2006)) was produced in Saccharomyces cerevisiae yeast, purified by metal affinity chromatography and anti-EGFR antibody affinity chromatography, and biotinylated on free amines using the sulfo-NHS biotinylation kit. The Fn3 yeast surface display libraries were pooled, grown in SD-CAA medium at 30° C., 250 rpm and display of Fn3 was induced in SG-CAA medium at 30° C., 250 rpm. Binders to streptavidin-coated magnetic Dynabeads were removed. One million biotinylated EGFR ectodomains were loaded on each often million magnetic beads and incubated with the remaining yeast. Beads were washed once with PBSA at 4° and beads with attached cells were grown for further selection. Remaining sorts were conducted with five million beads coated with one to two million ectodomains. After two sorts, full-length Fn3 clones were selected by FACS using the C-terminal c-myc epitope. Plasmid DNA was zymoprepped from the cells and mutagenized by error-prone PCR of the entire Fn3 gene or the BC, DE, and FG loops. Mutants were transformed into yeast by electroporation with homologous recombination and requisite shuffling of the loop mutants. The lead clones and their mutants were pooled for further cycles of selection and mutagenesis. Three rounds, each consisting of two binding sorts on beads, full-length clone isolation by FACS, and mutagenesis, were performed. Selection stringency was increased by additional washing and elevated temperature. In the fourth round, a single binding sort on magnetic beads was followed by a binding sort by FACS. Cells were incubated in 10 nM biotinylated ectodomain and mouse anti-c-myc antibody followed by fluorescein-conjugated anti-biotin antibody and R-phycoerythrin-conjugated anti-mouse antibody. Cells with the highest fluorescein:R-phycoerythrin ratio were collected. Three additional rounds of sorting and mutagenesis were performed with decreasing ectodomain concentrations during selections. Plasmids from binding populations were zymoprepped arid transformed into E. coli; transformants were grown, miniprepped, and sequenced.

The relative dominance of E4.2.1 and E4.2.2, as well as very similar mutants, initiated a campaign to identify additional unique clones. Binding populations from rounds two through five were sorted twice for binding to ectodomain in the presence of either ICR10, an antibody that competes with E4.2.2, or 528, an antibody that competes with E4.2.1. Unique clones were identified by sequence analysis.

Fn3 Production:

The Fn3 gene was digested with NheI and BamHI and transformed to a pET vector containing a HHHHHHKGSGK-encoding C-terminus (SEQ ID NO:______). The six histidines enable metal affinity purification, and the pentapeptide provides two additional amines for chemical conjugation. The plasmid was transformed into Rosetta (DE3) E. coli, which was grown in LB medium with 100 mg/L kanamycin and 34 mg/L chloramphenicol at 37°. Two hundred μL of overnight culture was added to 100 mL of LB medium, grown to an optical density of 0.2-1.5 units, and induced with 0.5 mM IPTG for 3-24 hours. Cells were pelleted, resuspended in lysis buffer (50 mM sodium phosphate, pH 8.0, 0.5M NaCl, 5% glycerol, 5 mM CHAPS, 25 mM imidazole, and 1× complete EDTA-free protease inhibitor cocktail), and exposed to four freeze-thaw cycles. The soluble fraction was clarified by centrifugation at 15,000 g for 10 min. and Fn3 was purified by metal affinity chromatography on TALON resin. Purified Fn3 was buffer exchanged into PBS and biotinylated with NHS-LC-biotin according to the manufacturer's instructions.

An Fn3-linker-Fn3 construct was produced by standard molecular cloning techniques. The resultant vector encodes for Fn3-EIDKPSQ-GSGGGSGGGKGGGGT-Fn3-EIDKPSQ-ELRS-HHHHHH in which the N-terminal Fn3 is bracketed by NheI and BamHI restriction sites and the C-terminal Fn3 is bracketed by KpnI and Sad sites. The reduced linker encodes a GSGT linker. The extended linker is GSGGGSGGGK-GGGSGGGNGGGSGGGGT (SEQ ID NO______). Protein was produced as for Fn3.

Affinity Titration:

A431 cells were washed in PBSA and incubated with various concentrations of biotinylated Fn3 on ice. The number of cells and sample volumes were selected to ensure excess Fn3 relative to EGFR. For some clones, this criterion necessitates very low cell density, which makes cell collection by centrifugation procedurally difficult. To obviate this difficulty, ‘bare’ yeast cells are added to the sample to enable effective cell pelleting during centrifugation. Cells were incubated on ice for sufficient time to ensure that the approach to equilibrium was at least 98% complete. Cells were then pelleted, washed with 1 mL PBSA, and incubated in PBSA with 10 mg/L streptavidin-R-phycoerythrin for 10-30 min. Cells were washed and resuspended with PBSA and analyzed by flow cytometry. The minimum and maximum fluorescence and the K_{d} value were determined by minimizing the sum of squared errors assuming a 1:1 binding interaction.

Epitope Conformational Sensitivity:

Yeast were grown and induced to display EGFR ectodomain, incubated at 4° C. or 80° C. for 30 minutes, and chilled on ice for 10 minutes. Cells were labeled with 40 nM biotinylated Fn3 and 300 nM mouse anti-c-myc antibody followed by streptavidin-R-phycoerythrin and AlexaFluor488-conjugated anti-mouse antibody. Fluorescence was quantified by flow cytometry. Binding (R-phycoerythrin) was normalized to full-length display (AlexaFluor488).

Competition:

Yeast displaying EGFR ectodomain or A431 cells were washed and incubated with initial competitor Fn3 or antibody for 30 minutes. Alternative competitor Fn3, antibody, or AlexaFluor488-conjugated EGF was then added and incubated for 30 minutes. Cells were washed and secondary reagent was added to detect the alternative competitor: fluorescein-conjugated anti-His antibody, streptavidin-R-phycoerythrin, R-phycoerythrin-conjugated anti-mouse antibody, and fluorescein-conjugated anti-rat antibody for Fn3, biotinylated Fn3, mouse antibodies, and rat ICR10, respectively. Cells were washed and analyzed by flow cytometry. Samples with and without initial competitor were compared to determine competition.

EGFR Fragment Labeling:

EGFR ectodomain fragments comprising amino acids 1-176, 294-543, and 302-503 were displayed on the yeast surface (Cochran et al., Journal of Immunological Methods, 287:147-158 (2004)). Cells were washed and incubated with 30 nM biotinylated Fn3 and mouse anti-c-myc antibody followed by streptavidin-R-phycoerythrin and AlexaFluor488-conjugated anti-mouse antibody. Cells were washed and analyzed by flow cytometry,

Fine Epitope Mapping:

A low mutation library of EGFR ectodomain, produced by Ginger Chao as described (Chao et al., Journal of Molecular Biology, 342:539-550 (2004)), was grown and induced. Yeast were labeled with biotinylated Fn3 and mouse anti-c-myc antibody followed by AlexaFluor647-conjugated streptavidin and AlexaFluor488-conjugated anti-mouse antibody. Cells were washed and analyzed by flow cytometry. Cells displaying full-length ectodomain (AlexaFluor488⁺) with reduced Fn3 binding (AlexaFluor647^{weak}) relative to unmutated ectodomain were collected, grown, and induced. Cells were then sorted twice for mutants of reduced binding with maintenance of foldedness as determined by binding to antibodies 199.12 or 225, which are conformationally sensitive (Cochran et al., Journal of Immunological Methods, 287:147-158 (2004)). Cells were labeled with biotinylated Fn3 and mouse 199.12 (for clones A, E, and E6.2.10) or mouse 225 (for clone D) anti-EGFR antibody followed by AlexaFluor647-conjugated streptavidin and R-phycoerythrin-conjugated anti-mouse antibody. Cells were washed and analyzed by flow cytometry. Cells displaying folded ectodomain (AlexaFluor488⁺) with reduced Fn3 binding (AlexaFluor647^{weak}) relative to unmutated ectodomain were collected, grown, and induced. Initial selections for clone C mapping yielded multiple glycine mutants and clones with multiple mutations. To improve the efficiency of folded mutants, analogous sorting was performed using the non-competitive domain III binder clone D for foldedness verification. Biotinylated clones C and D were independently complexed to AlexaFluor488- or AlexaFluor647-conjugated streptavidin and used to label the ectodomain library. Cells that exhibited binding to clone D but reduced clone C binding relative to wild-type ectodomain were collected. Selections for epitope mapping clone B yielded multiple mutants without a consistent location. The full-length ectodomains with reduced clone B binding were sorted for maintenance of clone D binding with a reduction in clone B binding.

Cell Culture:

All cells were grown at 37° C., 5% CO₂ in a humidified atmosphere. A431 cells were cultured in Dulbecco's modified Eagle medium (DMEM) supplemented with 10% fetal bovine serum (FBS). CHO cells transfected with a vector to express EGFR-green fluorescent protein were cultured in DMEM with 10% FBS, 1% sodium pyruvate, 1% non-essential amino acids, and 0.2 g/L G418, HeLa cells were cultured in Eagle's minimal essential medium with 10% FBS. hMEC cells were cultured in supplemented HuMEC medium. HT29 cells were cultured in McCoy's medium with 10% FBS. U87 cells were cultured in DMEM with 10% FBS, 1% sodium pyruvate, and 1% non-essential amino acids. Cells were detached for subculture or assay use with 0.25% trypsin and 1 mM EDTA. For serum starvation, medium was removed by aspiration, cells were washed with warm PBS, and fresh serum-free medium was added.

Downregulation Assays:

Cells were subcultured into 96-well plates, grown for 2 days, and serum starved for 12-18 h. Cells were treated with 20 nM Fn3-Fn3 or EGF for the indicated time. Medium was removed by aspiration and cells were washed with PBS, detached with trypsin/EDTA, and placed on ice for the remainder of the assay. Bound Fn3-Fn3 or ligand was removed by 5 min. acid strip with 0.2M acetic acid, 0.5 M NaCl. Cells were washed with PBSA and incubated in mouse 225 antibody followed by R-phycoerythrin-conjugated anti-mouse antibody. Cells were washed and analyzed by flow cytometry. Mean fluorescence was normalized to PBSA-treated control samples.

HEK Transfectants:

An EGFR expression vector built on the pcDNA3 vector was used as wild-type or modified by site-directed mutagenesis to introduce T654A, T669A, K721R, Y845F, 51045A/51046A, Y1068F, Y1148F, or Y1173F mutations. Mutation was verified by sequence analysis. HEK cells were grown to 1.2-1.5 million cells per mL and diluted to one million per mL. Miniprepped DNA and polyethyleneimine were independently diluted to 0.05 and 0.1 mg/mL in OptiPro medium and incubated at 22° for 15 min. Equal volumes of DNA and polyethyleneimine were mixed and incubated at 22° for 15 min. 1.2 mL of cells and 48 μL of DNA/polyethyleneimine mixture were added to a 24-well plate and incubated at 37°, 5% CO₂ with shaking for 24 h. One hundred μL aliquots of each transfection were transferred to a 96-well plate and grown for 24 h. A downregulation assay was performed as described.

In-Cell Western Blot:

A431 cells were cultured in 96-well plates, serum starved for 12-24 h, and treated with 20 nM Fn3-Fn3 or EGF. Cells were fixed for 10 min. by addition of an equal volume of 4% formaldehyde. Cells were washed and permeabilized with four washes of PBS with 0.1% Triton X100 and blocked in Odyssey blocking buffer for 2 h at 22° or overnight at 4°. Cells were incubated in 10 nM rabbit anti-phospho(S/T/Y) for 2 h at 22° or overnight at 4°. Four washes in PBS with 0.1% Tween20 were followed by 33 nM 800CW-conjugated anti-rabbit antibody and 180 nM ToPro3 and four additional washes. Plates were imaged at 700 nm and 800 nm. Antibody signal (800 nm) was normalized to DNA (700 nm) for each well.

Western Blot:

A431 cells were cultured in 24-well plates and serum starved for 16 h. For agonism assay, cells were treated with 20 nM Fn3-Fn3, antibody, or EGF for 15 min. For antagonism assay, cells were treated with Fn3, Fn3-Fn3, or antibody for 6 h followed by 1 nM EGF for 15 min. Medium was removed by aspiration and cells were washed twice with cold PBS and lysed for 5 min. in 50 μL of RIPA buffer with protease and phosphatase inhibitors and EDTA (Pierce). Lysates were clarified by centrifugation at 14,000 g for 15 min., separated by SDS-PAGE on a 12% BisTris gel, and blotted to nitrocellulose. Blots were blocked in 5% nonfat dry milk and labeled with 1:1000 anti-phosphoERK½ Y202/Y204 antibody (Cell Signaling, Danvers, Mass.) followed by peroxidase-conjugated anti-rabbit antibody. Blots were incubated in SuperSignal West Dura substrate and imaged. Blots were than washed extensively, labeled with rabbit anti-GAPDH antibody followed by peroxidase-conjugated anti-rabbit antibody, incubated with substrate and imaged. PhosphoERK½ Y202/Y204 labeling was normalized by GAPDH signal.

Quantitative Phosphoproteomics:

A431 cells were cultured in 12-well plates, serum starved for 16 h, and treated with 20 nM Fn3-Fn3, Fn3+Fn3, or EGF for 15 or 60 min. Medium was removed by aspiration and cells were washed with PBS and lysed in 8M urea with 1 mM Na₃ VO₄. Phosphoproteomic analysis was performed by Jason Neil of the Forest White lab (MIT). Lysates are digested to form peptides and labeled with iTRAQ reagents. Phosphotyrosine-containing peptides are isolated by immunoprecipitation with a pool of polyclonal anti-phosphotyrosine antibodies and phosphopeptides are enriched by immobilized metal affinity chromatography. Peptides are separated and analyzed by LC-MS/MS. Peptides are identified using MASCOT and relative abundance is determined by comparison of peak intensities.

Proliferation:

hMEC cells transfected with a vector for membrane-bound EGF ligand with a TGFα cytoplasmic tail (hMEC+TCT (Joslin et al., J. Cell Sci., 120:3688-99 (2007))) were obtained from Doug Lauffenburger (MIT). Eight thousand cells were plated into each well of a 96-well plate and incubated in 100 μL of medium with 20 nM agent for 48 h or 96 h. For 96 h samples, medium was supplemented with fresh agent at 48 h. Cell viability was quantified using the AlamarBlue assay (Invitrogen) according the manufacturer's instructions and normalized to PBSA-treated control.

Migration:

hMEC, hMEC+ECT, or hMEC+TCT cells were cultured in 96-well plates to confluent monolayers. Wounds were scratched into the monolayer using a pipette tip, and cells were washed with fresh medium and imaged on a Nikon confocal microscope with robotic stage. Cells were treated with 20 nM agent in 100 μL of medium, incubated for 24 h or 48 h, and imaged at identical fields of view. Migration was quantified as the average reduction in separation across the wound and normalized to PBSA-treated control.

Delivery:

Fn3 and Fn3-Fn3 were fluorophore-labeled on primary amines using DyLight633 NHS-ester (Pierce) according to the manufacturer's instructions and extensively desalted. HT29 cells were cultured in 96-well plates, serum starved, and incubated with 20 nM Fn3-(Fn3)-DyLight633 for 0-9 hours. Cells were detached using trypsin/EDTA, acid stripped in 0.2 M acetic acid, 0.5 M NaCl for 5 minutes and analyzed by flow cytometry.

Biotinylated Fn3 was incubated with streptavidin-NanoGold(1.4 nM)-AlexaFluor488 (Nanoprobes, Yaphank, N.Y.) at a 3:1 Fn3:streptavidin ratio. A431, HT29, and SW1222 cells were cultured in 96-well plates and treated with 20 nM complex for 12 h. Cells were detached using trypsin/EDTA, acid stripped in 0.2M acetic acid, 0.5M NaCl, and analyzed by flow cytometry.

### Example 3

### Production and Analysis of Ab-Fn3 Fusion Proteins

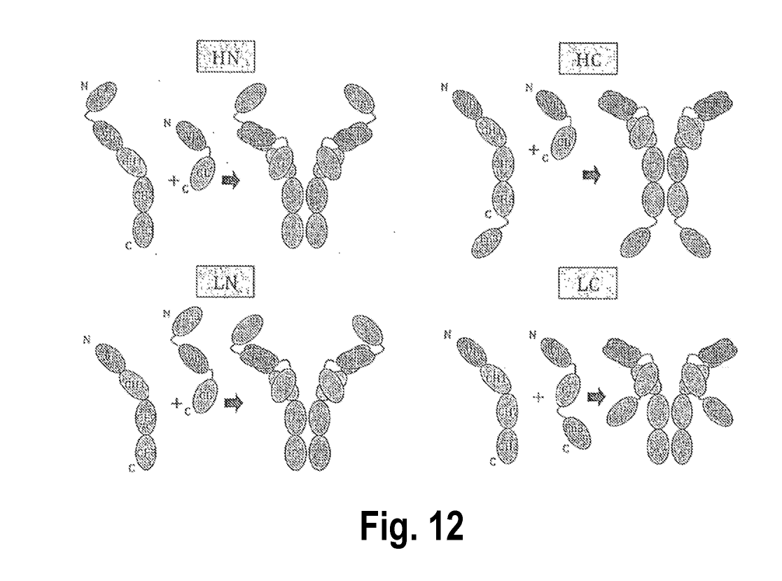

The modular constructs depicted in FIG. 12, which have configurations that can be assumed by the engineered proteins of the present invention, were secreted from HEK 293 cells co-transfected with havy and light chain expression plasmids derived from the gWiz vector. Secretions were harvested after eight days, purified via protein A affinity chromatography and concentrated in phosphate buffered saline. Yields ranged from 100-4000 μg/L depending on the antibody format and the fibronectin clone used.

The binding epitope of the 225 mAb from which the constructs were derived and the binding epitopes of various EGFR-binding fibronectins are shown in the Table below. Residues implicated in the binding of the EGFR targeted fibronectin clones were identified using yeast surface display based fine epitope mapping. The 225 epitope from the published crystal structure of the bound Fab fragment is also listed.

| Protein | EGFR Binding Domain | Epitope |
|---|---|---|
| Fn3 Clone A | I | L14, Q16, Y45, H69 |
| Fn3 Clone B | III | I327, V350M, F352V, W386R |
| Fn3 Clone C | III | I341, E376 |
| Fn3 Clone D | III | K430, S506 |
| Fn3 Clone E | III | T235, F335, V350, A351, |
| | | F352, T358 |
| mAb 225 | III | Q384, Q408, H409, K443, |
| | | K465, I467, N473 |

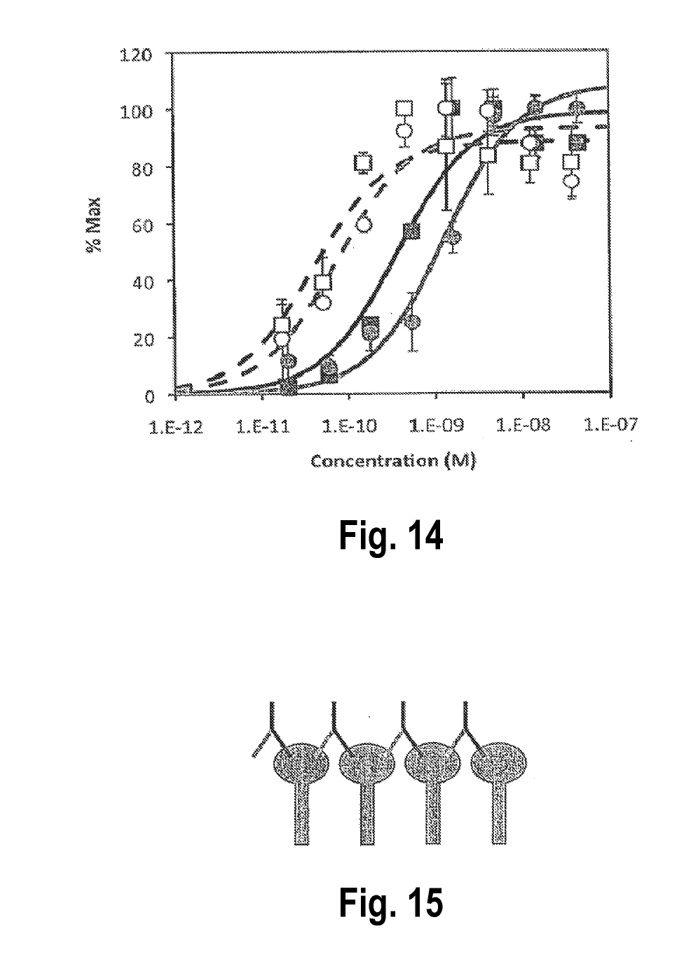

The interaction between the particular Ab-Fn3 fusion HN-D and its target antigen, EGFR, was characterized on the surface of A431 cells. As shown in FIG. 14, the affinity of the Ab-Fn3 fusion is an order of magnitude greater than that of the unmodified 225 antibody, both at endosomal pH (6.0) and physiological pH (7.4). The unconjugated 225 antibody and the Ab-Fn3 fusion HN-D were titrated on the surface of A431 cells at pH 6.0 and pH 7.4. A431 cells express 2.8×10⁶ EGFR per cell. The insensitivity of binding to pH reduction indicates that the engineered protein will remain bound to EGFR following internalization. The measured equilibrium dissociation constants for HN-D at pH 7.4 and 6.0 were 40 and 75 pM, respectively, compared to 370 and 1284 pM for mAb 225.

The advantage of targeting a cell-surface receptor such as EGFR with a multispecific (or heterobivalent) engineered protein is shown in FIG. 15. The presence of two non-competitive EGFR binding moieties enables receptor clustering. Clustering has been shown to abrogate EGFR recycling, thereby decreasing surface receptor expression and activation of downstream signaling pathways.

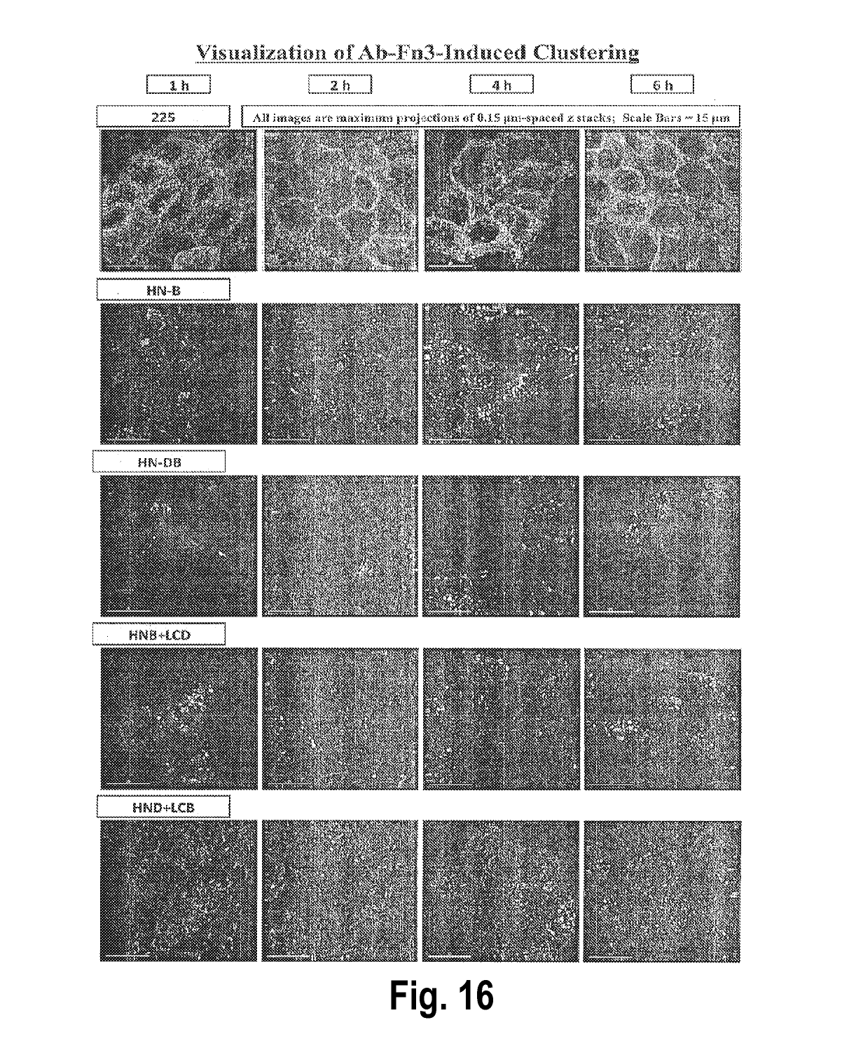

Deconvolution microscopy images show a dramatic change in receptor localization following Ab-Fn3 fusion treatment relative to 225 mAb treatment in two EGFR-expressing tumor cell lines, suggesting receptor clustering. To obtain visual evidence of multispecific antibody-induced clustering, A431 and HeLa cells were treated with fluorescently-labeled 225 or fluorescently-labeled FIN Ab-Fn3 fusion (containing fibronectin clone D) for 1, 2, 4, or 6 hours. Cells were then washed and imaged on a DeltaVision deconvolution microscope for comparison of EGFR localization. We observed a dramatic difference in receptor distribution following treatment with the multispecific construct compared to treatment with an unconjugated mAb (FIG. 16).

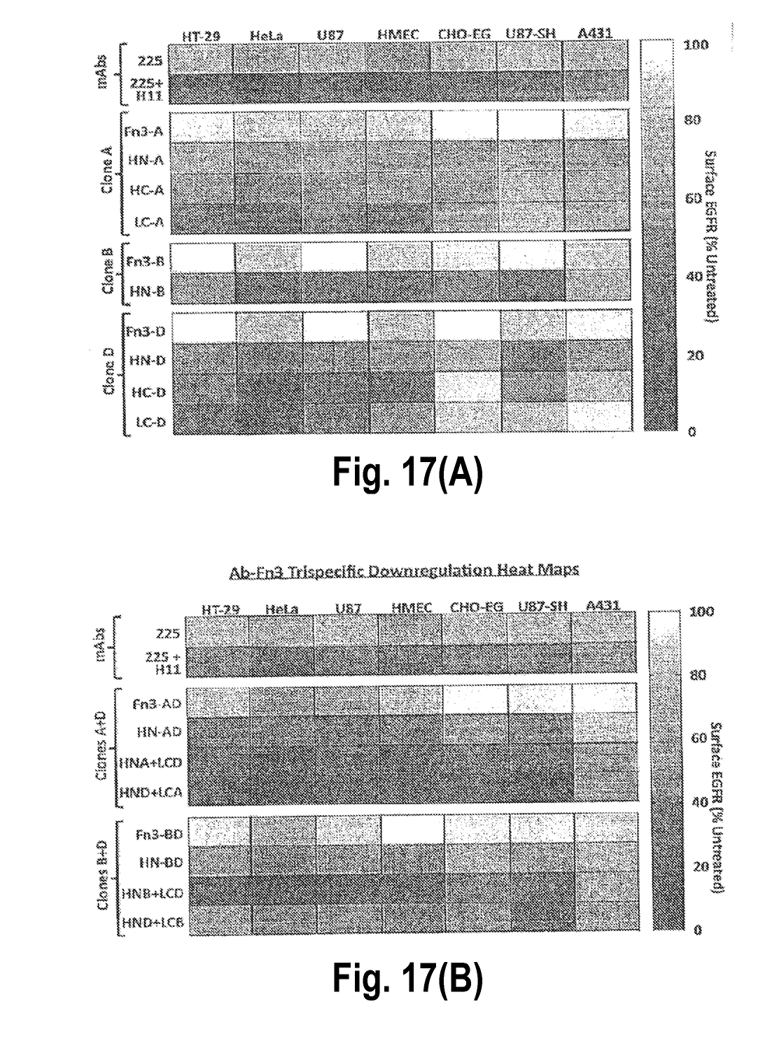

The results of studies of surface EGFR downregulation are shown in FIGS. 17(A) and (B). Seven EGFR expressing cell lines (listed in increasing order of EGFR expression) were treated with 20 nM antibody or antibody-fibronectin fusions for 13 hours at 37° C., allowing receptors to reach a new steady state level. Cells were then acid stripped, labeled with anti-EGFR antibody and fluorophore-conjugated secondary antibody, and analyzed via flow cytometry to quantify remaining surface receptor. Results are shown for the Ab-Fn3 fusions versus 225 and the potent 225+H11 mAb combination. The HN-B downregulates the most potently of all the single Fn3-containing fusions, but the bispecific compounds generally fail to potently downregulate receptor on EGFR-dense cell lines such as A431. In FIG. 7(B), the same seven EGFR expressing cell lines used in bispecific downregulation assays were treted with 20 nM antibody or antibody-fibronectin fusion for 13 hours at 37° C., allowing receptors to reach a new steady state level. Cells were then acid stripped, labeled with an anti-EGFR antibody and fluorophore-conjugated secondary antibody, and analyzed via flow cytometry to quantify remaining surface receptor relative to untreated cells. Results are shown for the Ab-Fn3 fusions versus 225 and the potent 225+H11 mAb combination. The trispecific constructs downregulate more potently than the bispecific constructions showin in FIG. 17(A) and that trispecific constructs with fibronectin moieties on both chains downregulate more effectively than those with both fibronectin moieties on the same chain. The most potent constructs (HNA+LCD, HND+LCA, and HNB+LCD) consistently reduce surface EGFR by 60-80%, performing as well or better than the 225+H11 combination on all cell lines tested.

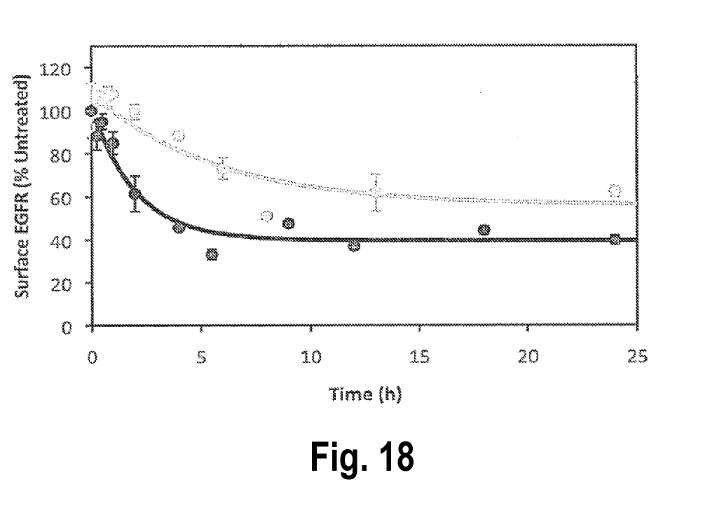

In other studies, we found that multispecific engineered proteins reproducibly induce synergistic downregulation in a host of EGFR-expressing cell lines. Seven EGFR-expressing cell lines (HT-29, HeLa, U87, HMEC, CHO-EG, U87-MGSH, and A431) were treated with 20 nM antibody or antibody-Fn fusion proteins for 13 hours at 37° C. They were then acid stripped, labeled with anti-EGFR antibody, and analyzed via flow cytometry to quantify the remaining surface receptor. The HN-D and LC-D constructs effectively downregulate EGFR nearly as effectively as the most potent combination of antibodies (225+H11) (FIG. 18).

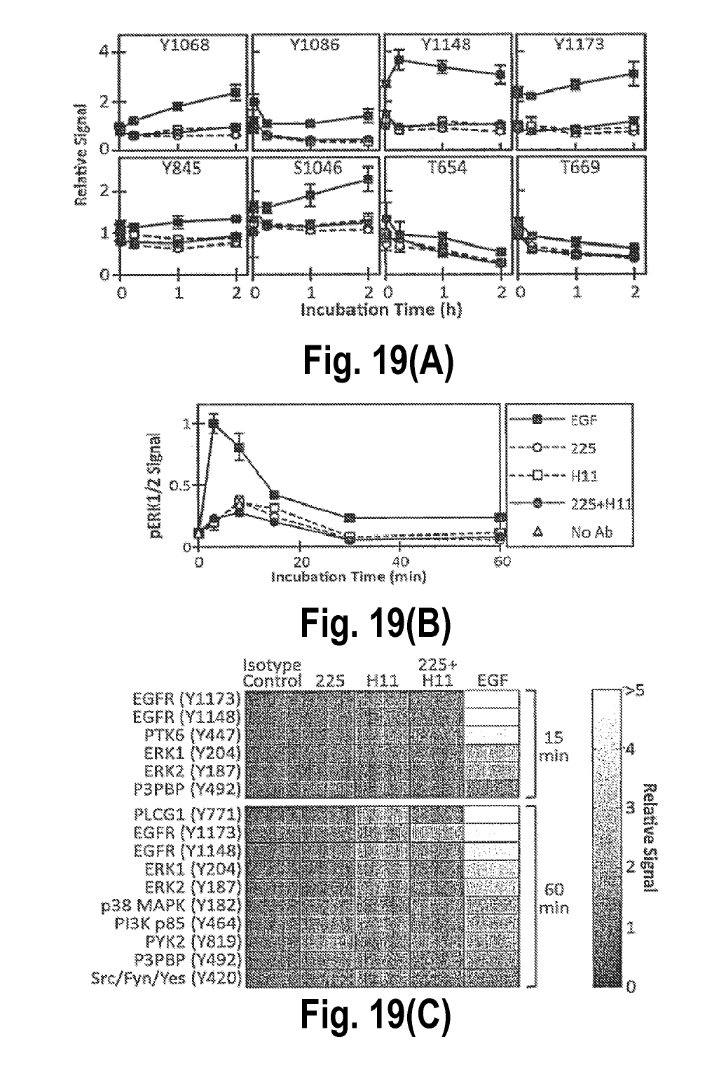

Further, in contrast to ligand stimulation, our engineered proteins reduced surface EGFR levels without activating EGFR or its downstream effectors. In-cell Western assays were performed on A431 cells for eight known EGFR phosphosites. Phosphoprotein fluorescence was normalized by DNA fluorescence and signal relative to that of untreated cells was plotted versus time (FIG. 19A). The timecourse of ERK ½ activation in A431 cells following mAb or EGF treatment was measured via bead-based immunoassay. Normalized phosphoprotein signal was plotted for cells treated with EGF, mAb 225, H11, and mAb 225+H11. An antibody-free control was also assessed (FIG. 19B). Serum-starved A431 cells were incubated with 225, H11, the 225+H11 combination, and EGF at 37° C. for 15 minutes or 60 minutes. EGF stimulation was held constant at 15 minutes for both screens. Cells were then lysed and relative protein phosphorylation was measured using an iTraq-based mass spectrometry screen. Phosphorylation levels were normalized by total protein content and signal strength relative to that in cells treated with an isotype control mAb is presented for MAPK and P13K pathway components. Repetition of the 60 minute screen yielded consistent results for proteins identified in both cohorts. Common downregulation profiles and receptor localization following combination mAb and Ab-Fn3 fusion treatment suggest that Ab-Fn3 constructs will not agonize EGFR signaling (FIG. 19C) (Spangler et al., Proc. Natl. Acad. Sci. USA, 107:13252-13257, 2010).

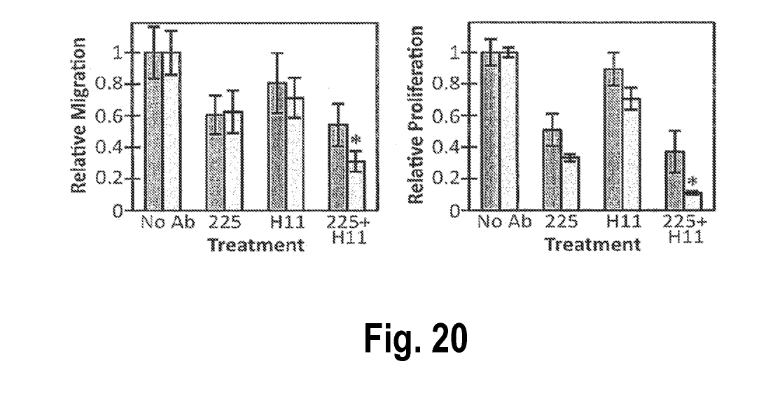

With respect to cell behavior, our studies have shown that combination antibody treatment protein selectively and significantly reduces migration and proliferation of cells that secrete high amounts of autocrine ligand (ECT) compared to treatment with the Ab (225 mAb) alone. We infer that Ab-Fn3 fusion-induced downregulation operates through a similar clustering mechanism and would thus inhibit migration and proliferation of ECT cells. Cell migration and proliferation of HMEC and autocrine EGF-secreting ECT cells were assessed using the scratch and MTT assays, respectively. For migration assays, monolayers were wounded and subsequently incubated with mAb 225, H11, and 225+H11 for 24 hours at 37° C. A “no antibody” incubation was also performed as a control. Relative migration was measured as fractional wound replenishment compared to that of the untreated control (FIG. 20). For proliferation assays, cells were treated with the specified mAbs for 72 hours at 37° C. Relative proliferation was assessed as viable cell abundance compared to that of untreated cells (FIG. 20). From the similarities between EGFR responses to combination mAb and Ab-Fn3 fusion treatment, we predict that Ab-Fn3 fusions will evoke similar responses in HMEC and TCT cells (Spangler et al., supra).

We are currently conducting pre-clinical studies of Ab-Fn3 fusions in mice with A431 tumor xenografts. In clinical trials, the patient population could include patients who are resistant to cetuximab therapy (and the present methods encompass methods of treatment for patients who are resistant to treatment with a target-specific protein scaffold alone).

Materials and methods used in the studies presented in Example 3 follow in the paragraphs below.

Cell Lines and Antibodies.

The transfected CHO-EG, U87-MGSH, and ECT cell lines were established as described previously and all other lines were obtained from ATCC (Manassas, Va.). Cells were maintained in their respective growth media (from ATCC unless otherwise indicated): DMEM for A431, U87-MG, U87-MGSH, and CHO-EG cells, McCoy's Modified 5A media for HT-29 cells, EMEM for HeLa cells, and HuMEC Ready Medium (Invitrogen, Carlsbad, Calif.) for HMEC and ECT cells. U87-MG, U87-MGSH, and CHO-EG media were supplemented with 1 mM sodium pyruvate (Invitrogen) and 0.1 mM non-essential amino acids (Invitrogen) and transfected lines U87-MGSH and CHO-EG were selected with 0.3 mM Geneticin (Invitrogen). ATCC media was supplemented with 10% fetal bovine serum (FBS). 225 was secreted from the hybridoma cell line (ATCC). Unless otherwise noted, all washes were conducted in PBS containing 0.1% BSA and all mAbs were used at a concentration of 40 nM for single treatment and 20 nM each for combination treatment. EGF (Sigma, St. Louis, Mo.) was dosed at 20 nM. Trypsin-EDTA (Invitrogen) contains 0.05% trypsin and 0.5 mM EDTA.

Production of Ab-Fn3 Fusions via HEK Cell Transfection:

The human IgG1 heavy and light chains of each Ab-Fn3 fusion were inserted into the gWiz mammalian expression vector (Genlantis). Constructs were verified by sequence analysis. HEK 293F cells (Invitrogen) were grown to 1.2 million cells per mL and diluted to one million per mL. Miniprepped DNA and polyethyleneimine (Sigma) were independently diluted to 0.05 and 0.1 mg/mL in OptiPro medium and incubated at 22° C. for 15 minutes. Equal volumes of DNA and polyethyleneimine were mixed and incubated at 22° C. for 15 minutes. 500 mL of cells and 20 mL of DNA/polyethyleneimine mixture were added to a 2 L roller bottle and incubated at 37°, 5% CO₂ on a roller bottle adapter for seven days. The cell secretions were then centrifuged for 30 minutes at 15,000×g and the supernatant was filtered through a 0.22 μm bottle-top filter and purified via affinity column chromatography using protein A resin (Thermo Fisher Scientific, Waltham, Mass.). The eluted constructs were concentrated and transferred to PBS and then characterized by sodium dodecyl sulfate polyacrylamide gel electrophoresis (SDS-PAGE) analysis.

Affinity Titrations.

To characterize Ab-Fn3 binding affinities, A431 cells were trypsinized, washed in PBSA, and incubated with various concentrations of Ab-Fn3 in a 96-well plate on ice. The number of cells and sample volumes were selected to ensure at least tenfold excess Ab-Fn3 relative to EGFR. Cells were incubated on ice for sufficient time to ensure that the approach to equilibrium was at least 99% complete. Cells were then washed and labeled with 66 nM PE-conjugated goat anti-human antibody (Rockland Immunochemicals, Gilbertsville, Pa.) for 20 min on ice. After a final wash, plates were analyzed on a FACS Calibur cytometer (BD Biosciences, San Jose, Calif.). Cell pelleting was conducted at 1000×g. The minimum and maximum fluorescence and the K_{d} value were determined by minimizing the sum of squared errors assuming a 1:1 binding interaction (% Bound=[L]/([L]+K_{d}) where [L] is Ab-Fn3 concentration and K_{d} is the equilibrium dissociation constant of the Ab-Fn3 construct. Titrations were performed at both pH 6.0 (endosomal pH) and pH 7.4 (physiological pH).

Receptor Quantification.

Cells were serum starved for 12-16 h, washed, digested in trypsin-EDTA (20 min at 37° C.), neutralized with complete medium, and labeled with 20 nM 225 for 1 h on ice. They were then washed, labeled with 66 nM phycoerythrin (PE)-conjugated goat anti-mouse antibody (Invitrogen) for 20 min on ice, washed again, and subjected to quantitative flow cytometry on an EPICS XL cytometer (Beckman Coulter, Fullerton, Calif.). Receptor density was calculated based on a curve of identically labeled anti-mouse IgG-coated beads (Bangs Laboratories, Fishers, Ind.).

Deconvolution Microscopy.

mAb 225 and Ab-Fn3 fusion constructs were labeled with Alexa 488 using a fluorescent labeling kit (Invitrogen). A431 cells were plated at 50,000 per well in 8-well microscopy chambers and allowed to settle overnight. They were then incubated with the appropriate mAb or fusion construct for various time lengths at 37°, 5% CO₂. Wells were then washed and cells were resuspended in phenol red-free medium for imaging on a Delta Vision inverted deconvolution microscope. Deconvolution of 0.15 μm z-slices and image analysis were performed using the Softworx software package.

Receptor Downregulation Assays.

Cells were seeded at 5×10⁴ per well in 96-well plates, serum starved for 12-16 h, treated with the indicated mAbs or Ab-Fn3 fusions in serum-free medium, and incubated at 37° C. At each time point, cells were washed and treated with trypsin-EDTA for 20 min at 37° C. Trypsin was neutralized with medium (10% FBS) and cells were transferred to v-bottom plates on ice. They were then washed, acid stripped (0.2 M acetic acid, 0.5 M NaCl, pH 2.5), and washed again prior to incubation with 20 nM 225 for 1 h on ice to label surface EGFR. Cells were then washed and labeled with 66 nM PE-conjugated goat anti-mouse antibody (Invitrogen) for 20 minutes on ice. After a final wash, plates were analyzed on a FACS Calibur cytometer (BD Biosciences, San Jose, Calif.). Cell pelleting was conducted at 1000×g.

In-Cell Western Assays.

A431 cells were seeded at 4×10⁴ per well in 96-well plates and allowed to adhere for 24 hours. Following 12-16 hours of serum starvation, cells were treated with the designated mAbs in serum-free medium at 37° C. for the specified time length. All subsequent incubations were performed at room temperature. Cells were fixed for 20 minutes (PBS, 4% formaldehyde), permeabilized via four 5 minute incubations (PBS, 0.1% triton), blocked for 1 hour in Odyssey blocking buffer (Licor Biosciences, Lincoln, Nebr.), and labeled for 1 hour with 15 nM anti-phosphosite antibodies (Genscript, Piscataway, N.J.) in blocking buffer. Cells were then washed three times with PBST (PBS, 0.1% Tween-20) and labeled with 66 nM 800-conjugated goat anti-rabbit antibody (Rockland Immunochemicals) and 400 nM TO-PRO-3 DNA stain (Invitrogen) in blocking buffer for 30 min. After three final PBST washes, wells were aspirated dry for analysis on a Licor Odyssey Scanner (Licor Biosciences). Signal was normalized to cell abundance by dividing 800 (phosphoprotein) by 700 (TO-PRO-3) channel fluorescence.

Luminex Phosphoprotein Quantification Assays.

A431 cells seeded in 96-well plates at 3×10⁴ per well were allowed to settle for 24 hours prior to 12-16 h serum starvation. Cells were then incubated with the specified mAbs in serum-free medium at 37° C. At the indicated times, cells were lysed using the Bio-Plex cell lysis kit (Bio-Rad, Hercules, Calif.). Phosphorylated ERK½ abundance was quantified using the Luminex bead-based immunoassay, performed with the Bio-Plex Phospho-ERK½ (T202/Y204, T185/Y187) bead kit and the Bio-Plex Phosphoprotein Detection Reagent kit on the Bio-Plex 200 platform (Bio-Rad).

Global Phospho-Mass Spectrometry Screens.

1×10⁶ A431 cells per well were seeded in 6-well plates, grown to confluence, and incubated with the appropriate mAbs in serum-free medium at 37° C. for 15 or 60 minutes. Cells were washed once with chilled PBS and lysed at 4° C. (8 M urea, 1 mM Na₃VO₄). Protein concentration was measured via bicinchoninic acid assay (Pierce, Rockford, Ill.). Lysate reduction, alkylation, trypsin digestion, and peptide fractionation were performed as previously described. Samples were labeled separately with 8 isotopic iTRAQ reagents (Applied Biosystems, Foster City, Calif.) for 2 hours at room temperature, combined, and concentrated. Immunoprecipitation with pooled anti-phosphotyrosine antibodies (4G10 (Millipore, Billerica, Mass.), pTyr100 (Cell Signaling, Beverly, Mass.), and PT-66 (Sigma)) proceeded for 16 h at 4° C. using protein G agarose beads (Calbiochem, San Diego, Calif.) in IP buffer (100 mM Tris, 100 mM NaCl, 1% Nonidet P-40, pH 7.4). Phosphopeptide enrichment by IMAC and analysis and quantification of eluted peptides were conducted via ESI LC/MS/MS on an LTQ-Orbitrap (Thermo Fisher Scientific). Phosphopeptides were identified using Mascot analysis software and spectra were manually validated. Signal intensities were normalized by total protein levels and compared to isotype control treatment.

Migration Assays.

HMEC and ECT cells were seeded at 5×10⁴ per well in 96-well plates and grown to confluence. Monolayers were wounded with a pipet tip, washed with PBS, and placed in complete medium with the indicated mAbs. Scratch area was measured immediately and after a 24 hour incubation at 37° C. using Image J software analysis of images from a Nikon confocal microscope (Nikon Instruments, Melville, N.Y.). Percent migration was calculated as the fractional reduction in scratch area in the treated wells divided by that of the untreated wells.

Cell Proliferation Assays.

HMEC and ECT cells were seeded at 5×10³ per well in 96-well plates and allowed to adhere for 24 h. They were then treated with the indicated mAbs in complete medium and incubated at 37° C. for 72 hours. Cell viability (relative to an untreated control) was assessed using the [3-(4,5-dimethylthiazol-2-yl)-2,5-diphenyltetrazolium bromide] (MTT) assay (Invitrogen).

Statistical Analysis.

Heteroscedastic two-tailed student's t tests were performed on migration and proliferation assay results to compare combination and single mAb treatment.

A number of embodiments of the invention have been described. Nevertheless, it will be understood that various modifications may be made without departing from the spirit and scope of the invention.

What is claimed is:

1. An engineered protein comprising a first genetically modified fibronectin domain that binds a first epitope on a molecular target and a second genetically modified fibronectin domain that binds a second epitope on the target.

2. The engineered protein of claim 1, further comprising a linker between the first fibronectin domain and the second fibronectin domain.

3. The engineered protein of claim 2, wherein the linker is a polypeptide.

4-5. (canceled)

6. The engineered protein of claim 1, further comprising a heterologous protein.

7. The engineered protein of claim 6, wherein the heterologous protein is a target-specific protein scaffold.

8. The engineered protein of claim 7, wherein the target-specific protein scaffold is an immunoglobulin or a biologically active fragment or other variant thereof.

9. The engineered protein of claim 8, wherein the immunoglobulin is the antibody cetuximab or the antibody panitumumab.

10. The engineered protein of claim 7, wherein the target-specific protein scaffold is a designed ankyrin repeat protein, an anticalin, or an affibody.

11. The engineered protein of claim 1, further comprising an accessory sequence.

12. The engineered protein of claim 11, wherein the accessory sequence is an amino acid sequence that prolongs the circulating half-life of the genetically modified Fn domain or an engineered protein of which it is a part; an amino acid sequence that facilitates isolation or purification of the engineered protein; an amino acid sequence that facilitates the bond between one part of the engineered protein and another or between the engineered protein and another moiety; an imaging agent or an amino acid sequence that can be detected and thereby serves as a label, marker, or tag; or an amino acid sequence that is toxic.

13. The engineered protein of claim 12, wherein the amino acid sequence that prolongs the circulating half-life is an Fc region of an immunoglobulin, albumin, another plasma protein, or fragments or variants thereof of a length sufficient to prolong the circulating half-life of the engineered protein.

14. The engineered protein of claim 12, wherein the polypeptide that facilitates isolation or purification of the engineered protein is a green fluorescent protein (GFP), glutathione S-transferase (GST), c-myc, hemagglutinin, β galactosidase, or Flag™ tag (Kodak) sequence.

15. The engineered protein of claim 12, wherein the moiety is a therapeutic compound.

16. The engineered protein of claim 1, wherein the first fibronectin domain and the second fibronectin domain are identical within their constant regions.

17. The engineered protein of claim 1, wherein the first fibronectin domain and the second fibronectin domain are at least 80% identical.

18. The engineered protein of claim 1, wherein the first fibronectin domain and/or the second fibronectin domain is a tenth type III fibronectin domain.

19. The engineered protein of claim 1, wherein the first fibronectin domain and/or the second fibronectin domain comprises a human fibronectin sequence.

20. The engineered protein of claim 1, wherein the first fibronectin domain and/or the second fibronectin domain comprises clone A, clone B, clone C, clone D, or clone E.

21. The engineered protein of claim 1, wherein the target is a cellular receptor.

22. The engineered protein of claim 21, wherein the cellular receptor is a receptor tyrosine kinase of the ErbB, insulin, PDGF, FGF, VEGF, HGF, Trk, Eph, AXL, LTK, TIE, ROR, DDR, RET, KLG, RYK, or MuSK receptor family.

23. The engineered protein of claim 22, wherein the cellular receptor is an EGF receptor.

24. A nucleic acid comprising a sequence encoding the engineered protein of claim 1.

25. A vector comprising the nucleic acid sequence of claim 24.

26. The vector of claim 25, wherein the vector is a plasmid or a cosmid or other viral vector.

27. A cell ex vivo comprising the vector of claim 26.

28. A pharmaceutically acceptable composition comprising the engineered protein of claim 1.

29. A method of treating a patient who has cancer, the method comprising identifying a patient in need of treatment and administering to the patient a therapeutically effective amount of the pharmaceutically acceptable composition of claim 28, wherein the engineered protein specifically binds at least one epitope on a protein whose expression or activity is associated with the cancer.

30. An engineered protein comprising (a) a genetically modified fibronectin domain that specifically binds a first epitope on a receptor tyrosine kinase and (b) a heterologous protein that specifically binds a second epitope on the tyrosine kinase receptor.

31. The engineered protein of claim 30, wherein the first epitope and the second epitope are non-overlapping.

32. The engineered protein of claim 30, wherein the genetically modified fibronectin domain is a mutant of a type III fibronectin domain.

33-36. (canceled)

37. The engineered protein of claim 30, wherein the heterologous protein is a target-specific protein scaffold.

38-40. (canceled)

41. The engineered protein of claim 30, further comprising an accessory sequence.

42-47. (canceled)

48. A nucleic acid comprising a sequence encoding the engineered protein of claim 30.

49. A vector comprising the nucleic acid sequence of claim 48.

50. (canceled)

51. A cell comprising the vector of claim 49.

52. A pharmaceutically acceptable composition comprising the engineered protein of claim 30.

53. A method of treating a patient who has cancer, the method comprising identifying a patient in need of treatment and administering to the patient a therapeutically effective amount of the pharmaceutically acceptable composition of claim 52, wherein the engineered protein specifically binds at least one epitope on a protein whose expression or activity is associated with the cancer.

54-55. (canceled)
